(19) United States
(12) Patent Application Publication
(10) Pub. No.: US 2009/0227650 A1
(43) Pub. Date: Sep. 10, 2009

(54) Novel crystalline forms of Lestaurtinib

(75) Inventors: Stephen Bierlmaier, Thorndale, PA (US); Laurent Courvoisier, Thorndale, PA (US); Raymond Scott Field, West Chester, PA (US); R.Curtis Haltiwanger, West Chester, PA (US); Martin J. Jacobs, West Chester, PA (US); Robert E. McKean, Chester Springs, PA (US); Mehran Yazdanian, Philadelphia, PA (US)

Correspondence Address: Ross J. Oehler;CEPHALON, Inc., 41 MOORES ROAD, PO BOX 4011, FRAZER, PA, 19355, US

(73) Assignee: Cephalon, Inc., Frazer, PA (US)

(21) Appl. No.: 12/355,284

(22) Filed: Jan. 16, 2009

Related U.S. Application Data
(60) Provisional application No. 61/011,434, filed on Jan. 16, 2008.

(51) Int. Cl. A61K 31/407; C07D 245/04; A61P 35/02

(52) U.S. Cl. 514/410; 540/460

(57) ABSTRACT

Novel crystalline forms of lestaurtinib are described, including six co-crystal forms, nineteen solvate forms, three anhydrate forms and one hemihydrate form. Methods of their preparation and use are also described.

### FIELD OF THE INVENTION

This invention pertains to lestaurtinib-containing compositions, pharmaceutical compositions comprising lestaurtinib, processes to reproducibly make them and methods of treating patients using them.

### BACKGROUND OF THE INVENTION

Active pharmaceutical ingredients (API or APIs (plural)) in pharmaceutical compositions can be prepared in a variety of different forms. Such APIs can be prepared so as to have a variety of different chemical forms, including but not limited to chemical derivatives, solvates, hydrates, hemihydrates, co-crystals, anhydrous forms or salts. Such APIs can also be prepared to have different physical forms. For example, the APIs may be amorphous, may have different crystalline polymorphs, or may exist in different solvation or hydration states. By varying the form of an API, it is possible to vary the physical properties thereof. For example, crystalline polymorphs typically have different solubilities, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility. Accordingly, variation of the crystalline state of an API is one of many ways in which to modulate the physical properties thereof.

Lestaurtinib is a semi-synthetic, orally bioavailable receptor-tyrosine kinase inhibitor that has been shown to have therapeutic utility in treating diseases such as acute myeloid leukemia, chronic myeloid leukemia and acute lymphocytic leukemia. It is a synthetic derivative of K-252a, a fermentation product of Nonomurea longicatena, and belongs to a class of indolocarbazole alkaloids. Lestaurtinib, (CAS Registry No. 111358-88-4), also known as (9S-(9α,10β,12α))-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one, is represented by the structure (I):

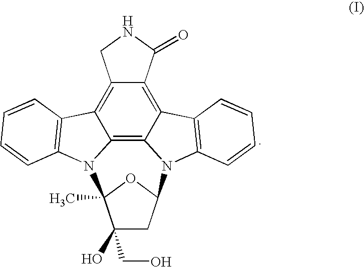

U.S. Pat. No. 4,923,986 describes lestaurtinib and utility thereof.

Different chemical forms of lestaurtinib can have different melting points, solubilities or rates of dissolution, which physical properties, either alone or in combination, can affect its bioavailability. Because knowledge of alternative chemical forms of lestaurtinib can provide guidance during clinical development, there is an existing need for identification of different and potentially improved forms of lestaurtinib, processes to reproducibly make them and methods of treating patients using them.

### SUMMARY OF THE INVENTION

It has now been found that co-crystalline, solvate, crystalline hemihydrate and crystalline anhydrous forms of lestaurtinib can be obtained, some of which can have improved properties as compared to the free form of lestaurtinib.

Accordingly, in one aspect, the present invention pertains to a co-crystal comprising lestaurtinib and a second component selected from the group consisting of maleic acid, malonic acid, oxalic acid, glutaric acid, hippuric acid and urea.

In another aspect, the co-crystal comprises lestaurtinib and maleic acid, and is characterized by a powder X-ray diffraction pattern comprising one or more peaks selected from the group consisting of about 7.56, 8.19, 16.47, 25.90 and 26.70 degrees 2-theta.

In another aspect, the co-crystal comprises lestaurtinib and malonic acid, and is characterized by a powder X-ray diffraction pattern comprising one or more peaks selected from the group consisting of about 7.99, 15.16, 16.04, 26.11 and 27.17 degrees 2-theta.

In another aspect, the co-crystal comprises lestaurtinib and oxalic acid, and is characterized by a powder X-ray diffraction pattern comprising one or more peaks selected from the group consisting of about 6.18, 7.44, 14.96, 20.19 and 25.78 degrees 2-theta.

In another aspect, the co-crystal comprises lestaurtinib and glutaric acid, and is characterized by a powder X-ray diffraction pattern comprising one or more peaks selected from the group consisting of about 14.10, 14.60, 25.12, 25.56 and 26.55 degrees 2-theta.

In another aspect, the co-crystal comprises lestaurtinib and hippuric acid, and is characterized by a powder X-ray diffraction pattern comprising one or more peaks selected from the group consisting of about 6.77, 14.23, 18.44, 20.61 and 25.19 degrees 2-theta.

In another aspect, the co-crystal comprises lestaurtinib and urea, and is characterized by a powder X-ray diffraction pattern comprising one or more peaks selected from the group consisting of about 14.63, 22.24, 25.19, 25.86 and 26.56 degrees 2-theta.

In a further aspect, the present invention pertains to a co-crystal comprising lestaurtinib and a second component selected from the group consisting of maleic acid, malonic acid, oxalic acid, glutaric acid, hippuric acid and urea for use as a pharmaceutical composition, comprising said co-crystal and one or more pharmaceutically acceptable excipients, diluents or carriers.

In yet another aspect, the present invention pertains to a pharmaceutical composition comprising Lestaurtinib Crystalline Form VI, Lestaurtinib Crystalline Form VII, Lestaurtinib Crystalline Form VIII, Lestaurtinib Crystalline Form IX, Lestaurtinib Crystalline Form X, Lestaurtinib Crystalline Form XI, Lestaurtinib Crystalline Form XII, Lestaurtinib Crystalline Form XIV, Lestaurtinib Crystalline Form XV, Lestaurtinib Crystalline Form XVI, Lestaurtinib Crystalline Form XX, Lestaurtinib Crystalline Form XXI, Lestaurtinib Crystalline Form XXII, Lestaurtinib Crystalline Form XXIII, Lestaurtinib Crystalline Form XXIV, Lestaurtinib Crystalline Form XXV, Lestaurtinib Crystalline Form XXVI, Lestaurtinib Crystalline Form XXVII, Lestaurtinib Crystalline Form XXVIII, or a mixture thereof.

In another aspect, the lestaurtinib is Lestaurtinib Crystalline Form VI. In another aspect, the lestaurtinib is Lestaurtinib Crystalline Form VII. In another aspect, the lestaurtinib is Lestaurtinib Crystalline Form VIII. In another aspect, the lestaurtinib is Lestaurtinib Crystalline Form IX. In another aspect, the lestaurtinib is Lestaurtinib Crystalline Form X. In another aspect, the lestaurtinib is Lestaurtinib Crystalline Form XI. In another aspect, the lestaurtinib is Lestaurtinib Crystalline Form XII. In another aspect, the lestaurtinib is Lestaurtinib Crystalline Form XIV. In another aspect, the lestaurtinib is Lestaurtinib Crystalline Form XV. In another aspect, the lestaurtinib is Lestaurtinib Crystalline Form XVI. In another aspect, the Lestaurtinib is Lestaurtinib Crystalline Form XX. In another aspect, the lestaurtinib is Lestaurtinib Crystalline Form XXI. In another aspect, the lestaurtinib is Lestaurtinib Crystalline Form XXII. In another aspect, the lestaurtinib is Lestaurtinib Crystalline Form XXIII. In another aspect, the lestaurtinib is Lestaurtinib Crystalline Form XXIV. In another aspect, the lestaurtinib is Lestaurtinib Crystalline Form XXV. In another aspect, the lestaurtinib is Lestaurtinib Crystalline Form XXVI. In another aspect, the lestaurtinib is Lestaurtinib Crystalline Form XXVII. In another aspect, the lestaurtinib is Lestaurtinib Crystalline Form XXVIII.

In still another aspect, the present invention pertains to a pharmaceutical composition comprising Lestaurtinib Crystalline Form VI, Lestaurtinib Crystalline Form VII, Lestaurtinib Crystalline Form VIII, Lestaurtinib Crystalline Form IX, Lestaurtinib Crystalline Form X, Lestaurtinib Crystalline Form XI, Lestaurtinib Crystalline Form XII, Lestaurtinib Crystalline Form XIV, Lestaurtinib Crystalline Form XV, Lestaurtinib Crystalline Form XVI, Lestaurtinib Crystalline Form XX, Lestaurtinib Crystalline Form XXI, Lestaurtinib Crystalline Form XXII, Lestaurtinib Crystalline Form XXIII, Lestaurtinib Crystalline Form XXIV, Lestaurtinib Crystalline Form XXV, Lestaurtinib Crystalline Form XXVI, Lestaurtinib Crystalline Form XXVII, Lestaurtinib Crystalline Form XXVIII, or a mixture thereof, further comprising amorphous lestaurtinib.

Another aspect of the present invention pertains to a solvate form of lestaurtinib that is Crystalline Form VI, Crystalline Form VII, Crystalline Form VIII, Crystalline Form IX, Crystalline Form X, Crystalline Form XI, Crystalline Form XII, Crystalline Form XIV, Crystalline Form XV, Crystalline Form XVI, Crystalline Form XX, Crystalline Form XXI, Crystalline Form XXII, Crystalline Form XXIII, Crystalline Form XXIV, Crystalline Form XXV, Crystalline Form XXVI, Crystalline Form XXVII, Crystalline Form XXVIII, or a mixture thereof.

In another aspect, the solvate is Crystalline Form VI, and is characterized by a powder X-ray diffraction pattern comprising one or more peaks selected from the group consisting of about 14.23, 17.69, 25.79, 26.59 and 27.12 degrees 2-theta.

In another aspect, the solvate is Crystalline Form VII, and is characterized by a powder X-ray diffraction pattern comprising one or more peaks selected from the group consisting of about 7.58, 17.75, 17.96, 21.48 and 22.08 degrees 2-theta.

In another aspect, the solvate is Crystalline form VIII, and is characterized by a powder X-ray diffraction pattern comprising one or more peaks selected from the group consisting of about 7.70, 11.94, 12.05, 17.11, 17.62 and 18.05 degrees 2-theta.

In another aspect, the solvate is Crystalline Form IX, and is characterized by a powder X-ray diffraction pattern comprising one or more peaks selected from the group consisting of about 7.79, 12.11, 15.55, 17.83 and 21.50 degrees 2-theta.

In another aspect, the solvate is Crystalline Form X, and is characterized by a powder X-ray diffraction pattern comprising one or more peaks selected from the group consisting of about 7.69, 11.99, 15.46, 17.79 and 17.96 degrees 2-theta.

In another aspect, the solvate is Crystalline Form XI, and is characterized by a powder X-ray diffraction pattern comprising one or more peaks selected from the group consisting of about 6.71, 14.44, 25.61, 26.51 and 27.80 degrees 2-theta.

In another aspect, the solvate is Crystalline Form XII, and is characterized by a powder X-ray diffraction pattern comprising one or more peaks selected from the group consisting of about 7.15, 18.18, 18.77, 21.27 and 24.98 degrees 2-theta.

In another aspect, the solvate is Crystalline Form XIV, and is characterized by a powder X-ray diffraction pattern comprising one or more peaks selected from the group consisting of about 7.75, 13.19, 14.21, 14.67, 17.55 and 25.13 degrees 2-theta.

In another aspect, the solvate is Crystalline Form XV, and is characterized by a powder X-ray diffraction pattern comprising one or more peaks selected from the group consisting of about 11.05, 13.91, 17.04, 17.09 and 25.59 degrees 2-theta.

In another aspect, the solvate is Crystalline Form XVI, and is characterized by a powder X-ray diffraction pattern comprising one or more peaks selected from the group consisting of about 8.12, 8.18, 10.31, 10.37 and 17.49 degrees 2-theta.

In another aspect, the solvate is Crystalline Form XX, and is characterized by a powder X-ray diffraction pattern comprising one or more peaks selected from the group consisting of about 7.73, 15.46, 17.95, 18.07 and 22.06 degrees 2-theta.

In another aspect, the solvate is Crystalline Form XXI, and is characterized by a powder X-ray diffraction pattern comprising one or more peaks selected from the group consisting of about 7.74, 12.19, 15.48, 18.18 and 22.27 degrees 2-theta.

In another aspect, the solvate is Crystalline Form XXII, and is characterized by a powder X-ray diffraction pattern comprising one or more peaks selected from the group consisting of about 11.04, 13.60, 15.74, 17.04, 25.58 degrees 2-theta.

In another aspect, the solvate is Crystalline Form XXIII, and is characterized by a powder X-ray diffraction pattern comprising one or more peaks selected from the group consisting of about 7.03, 14.06, 14.61, 15.04 and 26.31 degrees 2-theta.

In another aspect, the solvate is Crystalline Form XXIV, and is characterized by a powder X-ray diffraction pattern comprising one or more peaks selected from the group consisting of about 7.66, 14.40, 14.54, 14.78 and 25.32 degrees 2-theta.

In another aspect, the solvate is Crystalline Form XXV, and is characterized by a powder X-ray diffraction pattern comprising one or more peaks selected from the group consisting of about 5.52, 8.35, 10.88, 11.51 and 16.28 degrees 2-theta.

In another aspect, the solvate is Crystalline Form XXVI, and is characterized by a powder X-ray diffraction pattern comprising one or more peaks selected from the group consisting of about 7.14, 13.00, 14.27, 16.58, 18.02 and 19.94 degrees 2-theta.

In another aspect, the solvate is Crystalline Form XXVII, and is characterized by a powder X-ray diffraction pattern comprising one or more peaks selected from the group consisting of about 7.63, 9.80, 12.35, 15.27 and 21.93 degrees 2-theta.

In another aspect, the solvate is Crystalline Form XXVIII, and is characterized by a powder X-ray diffraction pattern comprising one or more peaks selected from the group consisting of about 9.86, 13.95, 18.52, 19.76 and 25.43 degrees 2-theta.

An additional aspect of the present invention pertains to a pharmaceutical composition comprising Lestaurtinib Crystalline Form XVII, Lestaurtinib Crystalline Form XVIII, Lestaurtinib Crystalline Form XIX, or a mixture thereof.

In another aspect, the lestaurtinib is Lestaurtinib Crystalline Form XVII. In another aspect, the lestaurtinib is Lestaurtinib Crystalline Form XVIII. In another aspect, the lestaurtinib is Lestaurtinib Crystalline Form XIX.

In still another aspect, the present invention pertains to a pharmaceutical composition comprising Lestaurtinib Crystalline Form XVII, Lestaurtinib Crystalline Form XVIII, Lestaurtinib Crystalline Form XIX, or a mixture thereof, further comprising amorphous lestaurtinib.

In yet another aspect, the present invention pertains to a crystalline anhydrate form of lestaurtinib that is Crystalline Form XVII, Crystalline Form XVIII, Crystalline Form XIX, or a mixture thereof.

In another aspect, the crystalline anhydrate is Crystalline Form XVII, and is characterized by a powder X-ray diffraction pattern comprising one or more peaks selected from the group consisting of about 7.90, 15.76, 19.63, 19.70 and 20.07 degrees 2-theta.

In another aspect, the crystalline anhydrate is Crystalline Form XVIII, and is characterized by a powder X-ray diffraction pattern comprising one or more peaks selected from the group consisting of about 7.76, 13.13, 15.64, 19.53 and 19.95 degrees 2-theta.

In another aspect, the crystalline anhydrate is Crystalline Form XIX, and is characterized by a powder X-ray diffraction pattern comprising one or more peaks selected from the group consisting of about 9.61, 11.07, 15.71, 17.07 and 18.39 degrees 2-theta.

In still another aspect, the present invention pertains to a pharmaceutical composition comprising Lestaurtinib Crystalline Form XIII. In another aspect, the present invention pertains to a pharmaceutical composition comprising Lestaurtinib Crystalline Form XIII, further comprising amorphous lestaurtinib.

In an additional aspect, the present invention pertains to a crystalline hemihydrate of lestaurtinib that is Crystalline Form XIII. In another aspect, the crystalline hemihydrate is characterized by a powder X-ray diffraction pattern comprising one or more peaks selected from the group consisting of about 6.89, 14.26, 14.73, 16.95 and 17.58 degrees 2-theta.

In a further aspect, the present invention pertains to a method of treating leukemia, comprising administering to a patient in need thereof a therapeutically effective amount of a preparation prepared from a composition according to any one of the foregoing forms. In a further aspect, the leukemia is selected from the group consisting of acute myeloid leukemia, chronic myeloid leukemia, acute lymphocytic leukemia and chronic lymphocytic leukemia.

The invention will now be described in further detail, by way of example only, with reference to the accompanying drawings.

### BRIEF DESCRIPTION OF THE DRAWINGS

FIG. 1 is an X-ray Powder Diffractogram (XRPD) of lestaurtinib free base;

FIG. 2 is a ¹H Nuclear Magnetic Resonance (NMR) spectrum for lestaurtinib free base;

FIG. 3 is a Differential Scanning Calorimetry (DSC) Thermogram and Thermo-Gravimetric Analysis (TGA) Thermogram overlay of lestaurtinib free base;

FIG. 4 is an X-ray Powder Diffractogram (XRPD) of the lestaurtinib:maleic acid co-crystal;

FIG. 5 is a ¹H Nuclear Magnetic Resonance (NMR) spectrum for the lestaurtinib:maleic acid co-crystal;

FIG. 6 is an X-ray Powder Diffractogram (XRPD) of the lestaurtinib:malonic acid co-crystal;

FIG. 7 is a ¹H Nuclear Magnetic Resonance (NMR) spectrum for the lestaurtinib:malonic acid co-crystal;

FIG. 8 is an X-ray Powder Diffractogram (XRPD) of the lestaurtinib:oxalic acid co-crystal;

FIG. 9 is a ¹H Nuclear Magnetic Resonance (NMR) spectrum for the lestaurtinib:oxalic acid co-crystal;

FIG. 10 is an X-ray Powder Diffractogram (XRPD) of the lestaurtinib:glutaric acid co-crystal;

FIG. 11 is a ¹H Nuclear Magnetic Resonance (NMR) spectrum for the lestaurtinib:glutaric acid co-crystal;

FIG. 12 is an X-ray Powder Diffractogram (XRPD) of the lestaurtinib:hippuric acid co-crystal;

FIG. 13 is a ¹H Nuclear Magnetic Resonance (NMR) spectrum for the lestaurtinib:hippuric acid co-crystal;

FIG. 14 is an X-ray Powder Diffractogram (XRPD) of the lestaurtinib:urea co-crystal;

FIG. 15 is a ¹H Nuclear Magnetic Resonance (NMR) spectrum for the lestaurtinib:urea co-crystal;

FIG. 16 is an X-ray Powder Diffractogram (XRPD) of lestaurtinib Crystalline Form VI;

FIG. 17 is a Differential Scanning Calorimetry (DSC) Thermogram and Thermo-Gravimetric Analysis (TGA) Thermogram overlay of lestaurtinib Crystalline Form VI;

FIG. 18 is an X-ray Powder Diffractogram (XRPD) of lestaurtinib Crystalline Form VII;

FIG. 19 is a Differential Scanning Calorimetry (DSC) Thermogram and Thermo-Gravimetric Analysis (TGA) Thermogram overlay of lestaurtinib Crystalline Form VII;

FIG. 20 is an X-ray Powder Diffractogram (XRPD) of lestaurtinib Crystalline Form VIII;

FIG. 21 is a Differential Scanning Calorimetry (DSC) Thermogram and Thermo-Gravimetric Analysis (TGA) Thermogram overlay of lestaurtinib Crystalline Form VIII;

FIG. 22 is an X-ray Powder Diffractogram (XRPD) of lestaurtinib Crystalline Form IX;

FIG. 23 is a Differential Scanning Calorimetry (DSC) Thermogram and Thermo-Gravimetric Analysis (TGA) Thermogram overlay of lestaurtinib Crystalline Form IX;

FIG. 24 is an X-ray Powder Diffractogram (XRPD) of lestaurtinib Crystalline Form X;

FIG. 25 is a Differential Scanning Calorimetry (DSC) Thermogram and Thermo-Gravimetric Analysis (TGA) Thermogram overlay of lestaurtinib Crystalline Form X;

FIG. 26 is an X-ray Powder Diffractogram (XRPD) of lestaurtinib Crystalline Form XI;

FIG. 27 is a Differential Scanning Calorimetry (DSC) Thermogram and Thermo-Gravimetric Analysis (TGA) Thermogram overlay of lestaurtinib Crystalline Form XI;

FIG. 28 is a ¹H Nuclear Magnetic Resonance (NMR) spectrum for lestaurtinib Crystalline Form XI;

FIG. 29 is an X-ray Powder Diffractogram (XRPD) of lestaurtinib Crystalline Form XII;

FIG. 30 is a Differential Scanning Calorimetry (DSC) Thermogram and Thermo-Gravimetric Analysis (TGA) Thermogram overlay of lestaurtinib Crystalline Form XII;

FIG. 31 is an X-ray Powder Diffractogram (XRPD) of lestaurtinib Crystalline Form XIII;

FIG. 32 is a Differential Scanning Calorimetry (DSC) Thermogram and Thermo-Gravimetric Analysis (TGA) Thermogram overlay of lestaurtinib Crystalline Form XIII;

FIG. 33 is an X-ray Powder Diffractogram (XRPD) of lestaurtinib Crystalline Form XIV;

FIG. 34 is a Differential Scanning Calorimetry (DSC) Thermogram and Thermo-Gravimetric Analysis (TGA) Thermogram overlay of lestaurtinib Crystalline Form XIV;

FIG. 35 is an X-ray Powder Diffractogram (XRPD) of lestaurtinib Crystalline Form XV;

FIG. 36 is a Differential Scanning Calorimetry (DSC) Thermogram and Thermo-Gravimetric Analysis (TGA) Thermogram overlay of lestaurtinib Crystalline Form XV;

FIG. 37 is an X-ray Powder Diffractogram (XRPD) of lestaurtinib Crystalline Form XVI;

FIG. 38 is an X-ray Powder Diffractogram (XRPD) of lestaurtinib Crystalline Form XVII;

FIG. 39 is a Differential Scanning Calorimetry (DSC) Thermogram and Thermo-Gravimetric Analysis (TGA) Thermogram overlay of lestaurtinib Crystalline Form XVII;

FIG. 40 is an X-ray Powder Diffractogram (XRPD) of lestaurtinib Crystalline Form XVIII;

FIG. 41 is an X-ray Powder Diffractogram (XRPD) of lestaurtinib Crystalline Form XIX;

FIG. 42 is a Differential Scanning Calorimetry (DSC) Thermogram and Thermo-Gravimetric Analysis (TGA) Thermogram overlay of lestaurtinib Crystalline Form XIX;

FIG. 43 is an X-ray Powder Diffractogram (XRPD) of lestaurtinib Crystalline Form XX;

FIG. 44 is an X-ray Powder Diffractogram (XRPD) of lestaurtinib Crystalline Form XXI;

FIG. 45 is an X-ray Powder Diffractogram (XRPD) of lestaurtinib Crystalline Form XXII;

FIG. 46 is a Differential Scanning Calorimetry (DSC) Thermogram and Thermo-Gravimetric Analysis (TGA) Thermogram overlay of lestaurtinib Crystalline Form XXII;

FIG. 47 is an X-ray Powder Diffractogram (XRPD) of lestaurtinib Crystalline Form XXIII;

FIG. 48 is a Differential Scanning Calorimetry (DSC) Thermogram and Thermo-Gravimetric Analysis (TGA) Thermogram overlay of lestaurtinib Crystalline Form XXIII;

FIG. 49 is an X-ray Powder Diffractogram (XRPD) of lestaurtinib Crystalline Form XXIV;

FIG. 50 is a Differential Scanning Calorimetry (DSC) Thermogram and Thermo-Gravimetric Analysis (TGA) Thermogram overlay of lestaurtinib Crystalline Form XXIV;

FIG. 51 is an X-ray Powder Diffractogram (XRPD) of lestaurtinib Crystalline Form XXV;

FIG. 52 is a Differential Scanning Calorimetry (DSC) Thermogram and Thermo-Gravimetric Analysis (TGA) Thermogram overlay of lestaurtinib Crystalline Form XXV;

FIG. 53 is an X-ray Powder Diffractogram (XRPD) of lestaurtinib Crystalline Form XXVI;

FIG. 54 is an X-ray Powder Diffractogram (XRPD) of lestaurtinib Crystalline Form XXVII;

FIG. 55 is a Differential Scanning Calorimetry (DSC) Thermogram and Thermo-Gravimetric Analysis (TGA) Thermogram overlay of lestaurtinib Crystalline Form XXVII;

FIG. 56 is an X-ray Powder Diffractogram (XRPD) of lestaurtinib Crystalline Form XXVIII;

FIG. 57 is a Differential Scanning Calorimetry (DSC) Thermogram and Thermo-Gravimetric Analysis (TGA) Thermogram overlay of lestaurtinib Crystalline Form XXVIII;

FIG. 58 is a ¹H Nuclear Magnetic Resonance (NMR) spectrum for lestaurtinib Crystalline Form XXVIII;

### DETAILED DESCRIPTION OF THE INVENTION

Different crystalline forms of a given drug have physical, pharmaceutical, physiological and biological properties which can sharply differ from one other. This invention pertains to co-crystalline, solvate, crystalline hemihydrate and crystalline anhydrous forms of lestaurtinib. It is meant to be understood that the term “lestaurtinib,” as used herein, without a designation of crystallinity or lack thereof, means a particular co-crystalline, solvate, crystalline hemihydrate or crystalline anhydrous form of lestaurtinib, lestaurtinib in solution or a mixture thereof.

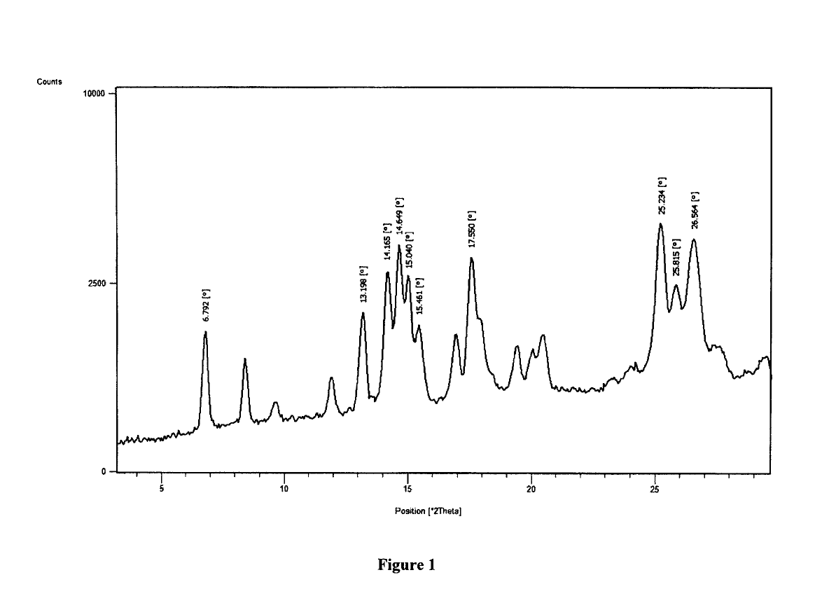

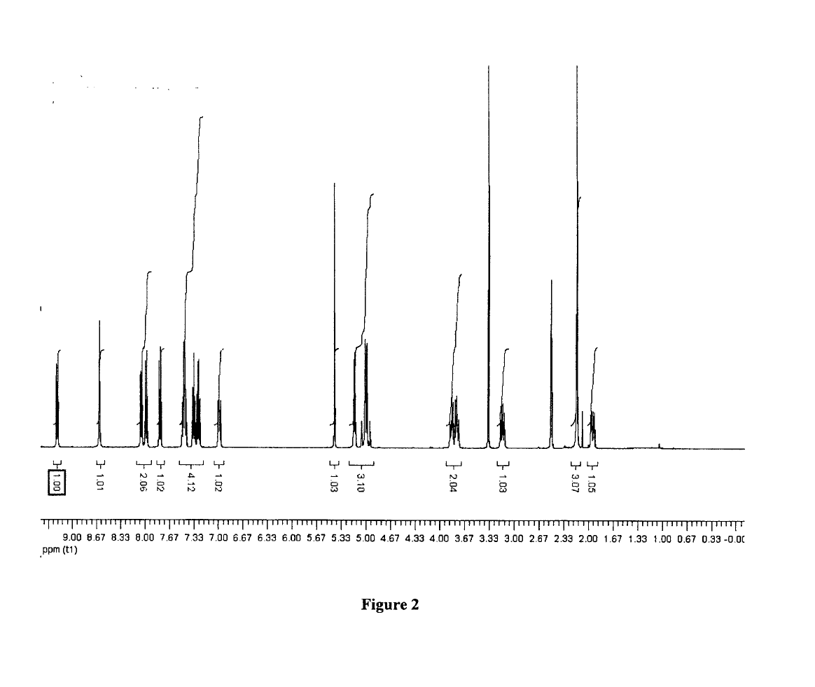

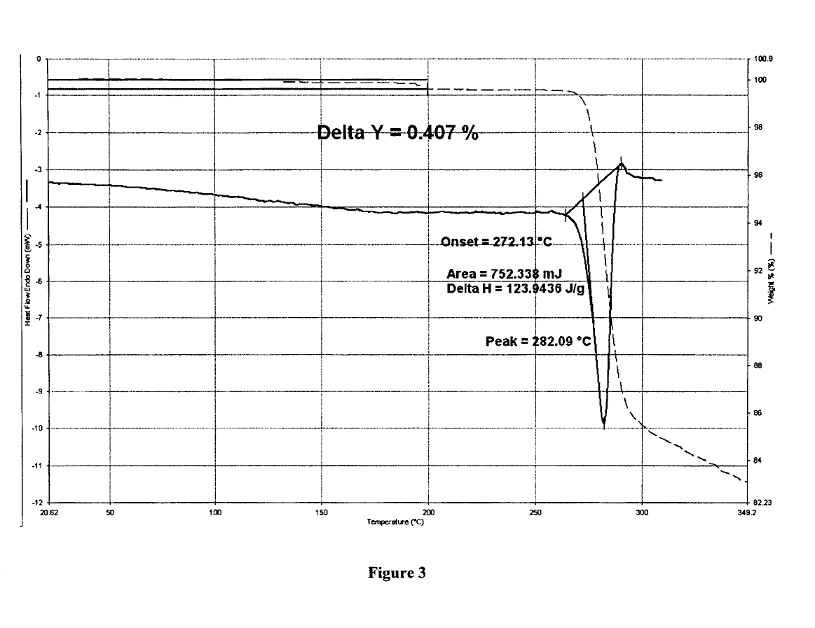

Crystalline lestaurtinib is characterized as a pale yellow powder of small particle size. The differential scanning calorimetry (DSC) scan for lestaurtinib shows an endotherm maximum at 282° C. Thermal gravimetric analysis (TGA) of the compound was done using a 5° C./min temperature ramp from room temperature to 350° C. The thermogram for lestaurtinib shows a 0.41% weight loss through 350° C. FIG. 1 shows the XRPD trace of lestaurtinib free base. FIG. 2 shows the ¹H NMR spectrum of lestaurtinib free base. FIG. 3 shows the DSC/TGA overlay of lestaurtinib free base. Representative XRPD peaks for lestaurtinib free base are listed in the following Table 1.

TABLE 1
Lestaurtinib free base XRPD peaks

| No. | Pos. [°2Th.] | d-spacing [Å] | Rel. Int. [%] |
|---|---|---|---|
| 1 | 6.79 | 13.02 | 36 |
| 2 | 8.41 | 10.51 | 21 |
| 3 | 11.93 | 7.42 | 12 |
| 4 | 13.20 | 6.71 | 41 |
| 5 | 14.17 | 6.25 | 70 |
| 6 | 14.65 | 6.05 | 91 |
| 7 | 15.04 | 5.89 | 63 |
| 8 | 15.46 | 5.73 | 32 |
| 9 | 16.94 | 5.23 | 25 |
| 10 | 17.55 | 5.05 | 78 |
| 11 | 17.97 | 4.94 | 31 |
| 12 | 19.42 | 4.57 | 17 |
| 13 | 20.01 | 4.44 | 14 |
| 14 | 20.47 | 4.34 | 23 |
| 15 | 25.23 | 3.53 | 100 |
| 16 | 25.82 | 3.45 | 46 |
| 17 | 26.56 | 3.36 | 86 |
| 18 | 27.58 | 3.23 | 11 |

The term “amorphous,” as used herein, means lacking a characteristic crystal shape or crystalline structure.

The term “anhydrate,” as used herein, refers to a chemical compound lacking the presence of water.

The term “anti-solvent,” as used herein, means a solvent in which a compound is substantially insoluble.

The term “co-crystal,” as used herein, means a crystalline composition comprised of two or more unique components, wherein no covalent chemical modification of the components occurs as a result of the co-crystal formation.

The term “crystalline,” as used herein, means having a regularly repeating arrangement of molecules or external face planes.

The term “crystalline composition,” as used in herein, refers to a solid chemical compound or mixture of compounds that provides a characteristic pattern of peaks when analyzed by x-ray powder diffraction; this includes, but is not limited to, polymorphs, solvates, hydrates, co-crystals, and desolvated solvates.

The term “hemihydrate,” has used herein, refers to a chemical compound for which the molecular ratio of water molecules to anhydrous compound is 1:2.

The term “isolating” as used herein, means separating a compound from a solvent, anti-solvent, or a mixture of solvent and anti-solvent to provide a solid, semisolid or syrup. This is typically accomplished by means such as centrifugation, filtration with or without vacuum, filtration under positive pressure, distillation, evaporation or a combination thereof. Isolating may or may not be accompanied by purifying during which the chemical, chiral or chemical and chiral purity of the isolate is increased. Purifying is typically conducted by means such as crystallization, distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

The terms “polymorph” or “polymorphism,” as used herein, refer to the occurrence of different crystalline arrangements for the same molecules.

The term “solute” as used herein, refers to a substance dissolved in another substance, usually the component of a solution present in the lesser amount.

The term “solution,” as used herein, refers to a mixture containing at least one solvent and at least one compound at least partially dissolved in the solvent.

The term “solvate,” as used herein, means a crystalline composition of variable stoichiometry formed by a solute and an organic solvent as defined herein.

The term “solvent,” as used herein, means a substance, typically a liquid, that is capable of completely or partially dissolving another substance, typically a solid. Solvents for the practice of this invention include, but are not limited to, water, acetic acid, acetone, acetonitrile, benzene, chloroform, carbon tetrachloride, dichloromethane, dimethylsulfoxide, 1,4-dioxane, ethanol, ethyl acetate, butanol, tert-butanol, N,N-dimethylacetamide, N,N-dimethylformamide, formamide, formic acid, heptane, hexane, isopropanol, methanol, methyl ethyl ketone (butanone), 1-methyl-2-pyrrolidinone, mesitylene, nitromethane, polyethylene glycol, propanol, 2-propanone, propionitrile, pyridine, tetrahydrofuran, toluene, xylene, mixtures thereof and the like.

The term “therapeutically effective amount,” as used herein, refers to the amount determined to be required to produce the physiological effect intended and associated with a given drug, as measured according to established pharmacokinetic methods and techniques, for the given administration route. Appropriate and specific therapeutically effective amounts can be readily determined by the attending diagnostician, as one skilled in the art, by the use of conventional techniques. The effective dose will vary depending upon a number of factors, including the type and extent of progression of the disease or disorder, the overall health status of the particular patient, the relative biological efficacy of the compound selected, the formulation of the active agent with appropriate excipients, and the route of administration.

Unless stated otherwise, percentages stated throughout this specification are weight/weight (w/w) percentages.

Mixtures comprising lestaurtinib and solvent may or may not have chemical and diastereomeric impurities, which, if present, may be completely soluble, partially soluble or essentially insoluble in the solvent. The level of chemical or diastereomeric impurity in the mixture may be lowered before or during isolation of lestaurtinib solvates by means such as distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

Mixtures of lestaurtinib and solvent, wherein the lestaurtinib is completely dissolved in the solvent may be prepared from a crystalline lestaurtinib, amorphous lestaurtinib or a mixture thereof.

It is meant to be understood that, because many solvents contain impurities, the level of impurities in solvents for the practice of this invention, if present, are at a low enough concentration that they do not interfere with the intended use of the solvent in which they are present. Solvents used were HPLC, reagent or USP grade and were used as received.

The invention provides methods of treating diseases and conditions in a patient comprising administering thereto a therapeutically effective amount of lestaurtinib. Accordingly, lestaurtinib is useful for treating a variety of therapeutic indications. For example, lestaurtinib is useful for the treatment of cancers such as carcinomas of the pancreas, prostate, breast, thyroid, colon and lung; malignant melanomas; glioblastomas; neuroectodermal-derived tumors including Wilm's tumor, neuroblastomas and medulloblastomas; and leukemias such as acute myeloid leukemia (AML), chronic myeloid leukemia (CML), acute lymphocytic leukemia (ALL), chronic lymphocytic leukemia (CLL); pathological conditions of the prostate such as prostatic hypertrophy or prostate cancer; carcinomas of the pancreas, such as pancreatic ductal adenocarcinoma (PDAC); hyperproliferative disorders such as proliferative skin disorders including actinic keratosis, basal cell carcinoma, squamous cell carcinoma, fibrous histiocytoma, dermatofibrosarcoma protuberans, hemangioma, nevus flammeus, xanthoma, Kaposi's sarcoma, mastocytosis, mycosis fungoides, lentigo, nevocellular nevus, lentigo maligna, malignant melanoma, metastatic carcinoma and various forms of psoriasis, including psoriasis vulgaris and psoriasis eosinophilia; and myeloproliferative disorders and related disorders associated with activation JAK2 and myeloproliferative disorders and related disorders including, but are not limited, to myeloproliferative diseases such as, for example, polycythemia vera (PV), essential thrombocythemia (ET), myelofibrosis with myeloid metaplasia (MMM), also called chronic idiopathic myelofibrosis (CIMF), unclassified myeloproliferative disorders (uMPDs), hypereosinophilic syndrome (HES), and systemic mastocytosis (SM). In a preferred aspect, the invention includes a method of treating acute myeloid leukemia (AML), and myeloproliferative disorders (MPDs) including chronic mylogenous leukemia (CML), polycythemia vera (PV), essential thrombocythemia (ET), chronic idiopathic myelofibrosis (CIMF/AMM), chronic eosinophilic leukemia (CEL), chronic neutrophilic leukemia (CNL), and hypereasinophilic syndrome (HEL). More preferably, the invention includes a method of treating acute myeloid leukemia (AML).

Lestaurtinib can be administered by any means that results in contact of the active agent with the agent's site of action in the body of the patient. Lestaurtinib can be administered by any conventional means available, either as an individual therapeutic agent or in combination with other therapeutic agents. Lestaurtinib is preferably administered to a patient in need thereof in therapeutically effective amounts for the treatment of the diseases and disorders described herein.

Therapeutically effective amounts of lestaurtinib can be readily determined by an attending diagnostician by use of conventional techniques. The effective dose can vary depending upon a number of factors, including type and extent of progression of the disease or disorder, overall health of a particular patient, biological efficacy of the lestaurtinib, formulation of the lestaurtinib, and route of administration of the forms of lestaurtinib. Lestaurtinib can also be administered at lower dosage levels with gradual increases until the desired effect is achieved.

As used herein, the term “about”, when referring to dosage or temperature, refers to a range of values from ±10% of a specified value. For example, the phrase “about 50 mg” includes ±10% of 50 or from 45 to 55 mg.

Typical dose ranges of lestaurtinib in its free form comprise from about 0.01 mg/kg to about 100 mg/kg of body weight per day. Alternatively, a typical dose range of free form lestaurtinib comprises from about 0.01 mg/kg to 10 mg/kg of body weight per day. Daily doses for adult humans includes about 20, 25, 30, 35, 40, 45, 50, 55, 60, 65, 70, 75, 80, 90, 100, 120, 140, 160 and 200 mg and an equivalent dose for a human child. Lestaurtinib can be administered in one or more unit dose forms and can also be administered one to four times daily, including twice daily (BID). Examples of free form lestaurtinib administration comprise from about 1 to about 400 mg administered one to four times a day; from about 10 mg to about 200 mg BID; from 20-80 mg BID; from 60-100 mg BID, and; from about 40, 60, 80, or 100 mg BID.

Dosages of free form lestaurtinib can also be in the form of liquids or suspensions in a concentration of between 15 to 25 mg/mL, 16 mg/mL or 25 mg/mL. The liquid or suspension dosage forms of free form lestaurtinib can include the equivalent of the doses (mg) described above. For example, dosages of free form lestaurtinib can include 1 to 5 mL of the 25 mg/mL solution, or 1, 1.2, 1.4, 1.6, 1.8, 2, 2.2, 2.4, 2.6, 2.8, 3, 3.2, 3.4, 3.6, 3.8, or 4 mL of the 25 mg/mL solution, wherein a 60 mg dose of free form lestaurtinib can be provided in 2.4 mL of solution, an 80 mg dose of free form lestaurtinib can be provided in 3.2 mL of solution and a 100 mg dose of free form lestaurtinib can be provided in 4 mL of solution. Additionally, a 20 mg dose of free form lestaurtinib can be provided with a 1.25 mL of a 16 mg/mL solution.

The daily dose of free form lestaurtinib can range from 1 mg to 5 mg/kg (normalization based on a mean body weight close to 65 kg). For example, a daily dose of free form lestaurtinib is from about 1 to 3 mg/kg or from about 1.2 to 2.5 mg/kg, or about 1.2, 1.4, 1.6, 1.8, 2, 2.2, 2.4, 2.6, 2.8 or 3 mg/kg. In an alternate method of describing an effective dose, an oral unit dose of free form lestaurtinib is one that is necessary to achieve a blood serum level of about 0.05 to 20 μg/mL or from about 1 to 20 μg/mL in a patient.

Lestaurtinib can be formulated into pharmaceutical compositions by mixing the forms with one or more pharmaceutically acceptable excipients. It is meant to be understood that pharmaceutical compositions include any form of lestaurtinib or any combination thereof.

The term “pharmaceutically acceptable excipients,” as used herein, includes any and all solvents, dispersion media, coatings, antibacterial and antifungal agents, isotonic and absorption delaying agents and the like. The use of such media and agents for pharmaceutical active substances is well known in the art, such as in Remington: The Science and Practice of Pharmacy, 20^{th} ed.; Gennaro, A. R., Ed.; Lippincott Williams & Wilkins: Philadelphia, Pa., 2000. Except insofar as any conventional media or agent is incompatible with the active ingredient, its use in the therapeutic compositions is contemplated. Supplementary active ingredients can also be incorporated into the compositions.

Excipients for preparation of compositions comprising forms of lestaurtinib to be administered orally include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water and mixtures thereof. Excipients for preparation of compositions comprising forms of lestaurtinib to be administered ophthalmically or orally include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for preparation of compositions comprising forms of lestaurtinib to be administered osmotically include, for example, chlorofluoro-hydrocarbons, ethanol, water and mixtures thereof. Excipients for preparation of compositions comprising forms of lestaurtinib to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for preparation of compositions comprising forms of lestaurtinib to be administered rectally or vaginally include, for example, cocoa butter, polyethylene glycol, wax and mixtures thereof.

Dosage forms of lestaurtinib and compositions comprising lestaurtinib depend upon the route of administration. Any route of administration is contemplated, including oral, mucosal (e.g. ocular, intranasal, pulmonary, gastric, intestinal, rectal, vaginal and uretheral) or parenteral (e.g. subcutaneous, intradermal, intramuscular, intravenous, or intraperitoneal.

Pharmaceutical compositions are most preferably administered orally, preferably in forms such as tablets, capsules, powders, pills, liquids/suspensions or gels/suspensions or emulsions, lyophillizates and all other different forms described in patents and applications mentioned herein, more preferably as tablets, capsules and liquids/suspensions or gels/suspensions. The administration vehicle can comprise one or more pharmaceutically acceptable carriers that are likely to ensure the solid state or crystalline form's stability (e.g. a suspension in oil).

Lestaurtinib can be formulated as a variety of pharmaceutical compositions and dosage forms, such as those described in U.S. Pat. Nos. 6,200,968 and 6,660,729 and PCT Publication No. 04/037928, each of which is incorporated herein by reference. In particular, the lestaurtinib can be formulated as microemulsions or dispersions.

### EXAMPLES

Crystalline forms of lestaurtinib can be made by synthetic chemical processes, examples of which are shown herein below. It is meant to be understood that the order of the steps in the processes may be varied, that reagents, solvents and reaction conditions may be substituted for those specifically mentioned, and that moieties susceptable to undesired reaction may be protected and deprotected, as necessary.

The following examples are presented to provide what is believed to be the most useful and readily understood description of procedures and conceptual aspects of this invention.

### Analytical Methods

The following methods were used to characterize the compounds described herein.

X-ray powder diffraction (XRPD) patterns for the samples were acquired on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consisted of a single Gobel multilayer mirror coupled with a pinhole collimator of 0.3 mm. Beam divergence, i.e. the effective size of the X-ray beam on the sample, was approximately 4 mm. A 0-0 continuous scan mode was employed with a sample to detector distance of 20 cm which provided an effective 20 range of 3.2-29.8°. A typical exposure time of a sample was 120 seconds.

Samples run under ambient conditions were prepared as flat plate specimens using powder as received without grinding. Approximately 1-2 mg of the sample was lightly pressed on a glass slide to obtain a flat surface. Samples run under non-ambient conditions were mounted on a silicon wafer with heat conducting compound. The sample was then heated to the appropriate temperature at ca. 20° C./minute and subsequently held isothermally for ca 1 minute before data collection was initiated.

Powder XRD patterns were also recorded on a PANalytical X'Pert Pro diffractometer equipped with an X'celerator detector using Cu Kα radiation at 40 kV and 40 mA. Kα radiation is obtained with a highly oriented crystal (Ge111) incident beam monochromator. A 10 mm beam mask, and fixed (¼°) divergence and anti-scatter (⅛°) slits were inserted on the incident beam side. A fixed 0.10 mm receiving slit was inserted on the diffracted beam side. The X-ray powder pattern scan was collected from ca. 2 to 40° 20 with a 0.0080° step size and 96.06 sec counting time which resulted in a scan rate of approximately 0.5°/min. The sample was spread on a glass plate or a silicon zero background (ZBG) plate for the measurement. The sample was rotated at 4°/min on a PANalytical PW3064 Spinner.

Samples with ca. 500 mg of available material were back-loaded into a sample holder ring and mounted on a common bottom plate. The resulting X-ray patterns possessed minimal height variation and were typically of higher quality. The resulting XRPD patterns were evaluated and reports were prepared using the PANalytical High Score plus software package.

The crystals chosen were coated with paratone oil and flash frozen on an Oxford diffraction CCD diffractometer (Xcalibur S, with a Sapphire detector). Data were collected with standard area detector techniques. The structures were solved and refined with the SHELXTL package. To determine the unit cell at room temperature and to check the agreement of the of the single crystal parameters against the measured XRPD pattern, Reitveld refinement calculations were carried out with default (as set by PANalytical) refinement conditions. None of the atomic parameters were refined in the Reitveld calculations.

Variable Temperature (VT) and Low Humidity XRPD experiments were performed with an Anton Paar TTK450 chamber that was computer controlled for temperature only. The humidity in the chamber could be effectively reduced to very low RH conditions by flowing nitrogen gas through the TTK450 chamber.

Thermal curves were acquired using a Perkin-Elmer Sapphire DSC unit equipped with an autosampler running Pyris software version 6.0 calibrated with Indium prior to analysis. Solid samples of 1-11 mg were weighed into 20 μL aluminum open samples pans. The DSC cell was then purged with nitrogen and the temperature heated from 0° to 300° C. at 10° C./min.

Thermal curves were also acquired using a Perkin-Elmer Pyris 1 TGA unit running Pyris software version 6.0 calibrated with calcium oxalate monohydrate. TGA samples between 1-15 mg were monitored for percent weight loss as heated from 25° to 400° C. at 10° C./min in a furnace purged with Nitrogen at ca. 50 mL/min.

All NMR spectra were collected on a Bruker 400 MHz equipped with an autosampler. Samples were prepared in d₆-DMSO, unless otherwise stated.

Lestaurtinib was prepared as described in U.S. Pat. No. 4,923,986.

### Example 1

### Lestaurtinib—Maleic Acid Co-Crystal

100 mg of lestaurtinib was ground with 1 mole equivalent (26.4 mg) of maleic acid in a Copley ball mill. The mixture was ground during three 30 minute periods at increasing oscillation frequencies (5 Hz, 10 Hz and 15 Hz, respectively).

8 mg of the ground sample was weighed into a small, screw top vial. 200 μl of dichloromethane (DCM) was added and the vial was subjected to heat/cool cycles between ambient and 50° C. (4 hours hot, 4 hours cool) for 24 hours.

Solid was isolated by filtration and dried under ambient conditions for 1 hour before analysis.

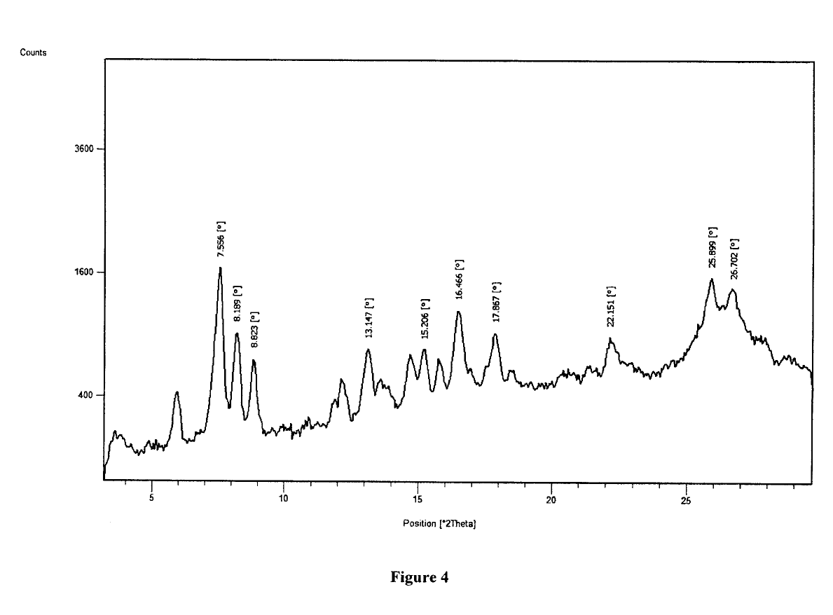

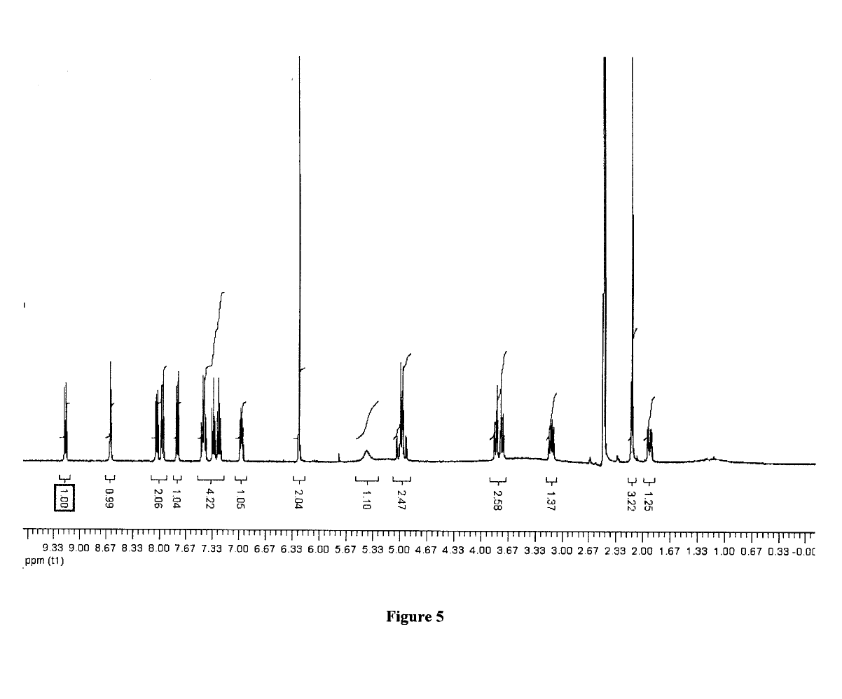

FIG. 4 shows the XRPD pattern for the lestaurtinib:maleic acid co-crystal. FIG. 5 shows the ¹H NMR spectrum for the lestaurtinib:maleic acid co-crystal. Representative XRPD peaks for the lestaurtinib:maleic acid co-crystal are listed in the following Table 2.

TABLE 2
Lestaurtinib: maleic acid XRPD peaks

| No. | Pos. [°2Th.] | d-spacing [Å] | Rel. Int. [%] |
|---|---|---|---|
| 1 | 5.95 | 14.86 | 15 |
| 2 | 7.56 | 11.70 | 100 |
| 3 | 8.19 | 10.80 | 45 |
| 4 | 8.82 | 10.02 | 29 |
| 5 | 12.17 | 7.27 | 16 |
| 6 | 13.15 | 6.73 | 30 |
| 7 | 14.68 | 6.03 | 21 |
| 8 | 15.21 | 5.83 | 22 |
| 9 | 15.78 | 5.62 | 14 |
| 10 | 16.47 | 5.38 | 47 |
| 11 | 17.87 | 4.96 | 29 |
| 12 | 22.15 | 4.01 | 22 |
| 13 | 25.90 | 3.44 | 65 |
| 14 | 26.70 | 3.34 | 55 |

In a preferred aspect of the present invention, representative XRPD peaks for the lestaurtinib:maleic acid co-crystal comprise one or more peaks selected from the group consisting of about 7.56, 8.19, 8.82, 13.15, 15.21, 16.47, 17.87, 22.15, 25.90 and 26.70 degrees 2-theta. In an even more preferred aspect, representative XRPD peaks for the lestaurtinib:maleic acid co-crystal comprise one or more peaks selected from the group consisting of about 7.56, 8.19, 16.47, 25.90 and 26.70 degrees 2-theta.

### Example 2

Lestaurtinib—Malonic Acid Co-Crystal

100 mg of lestaurtinib was ground with 1 mole equivalent (23.7 mg) of malonic acid in a Copley ball mill. The mixture was ground during three 30 minute periods at increasing oscillation frequencies (5 Hz, 10 Hz and 15 Hz, respectively).

8 mg of the ground sample was weighed into a small, screw top vial. 200 μl of dichloromethane (DCM) was added and the vial was subjected to heat/cool cycles between ambient and 50° C. (4 hours hot, 4 hours cool) for 24 hours.

Solid was isolated by filtration and dried under ambient conditions for 1 hour before analysis.

In an alternative procedure, 80 mg of lestaurtinib with 1 mole equivalent (19.1 mg) of malonic acid was slurried in 1.8 mL of DCM. The samples were subjected to 6 cycles of maturation. Each cycle consisted of: warming over 1 hour to 50° C., holding at 50° C. for 4 hours, cooling over 3 hours (0.25° C./min) to 5° C., and holding at 5° C. for 4 hours. Solid was isolated by suction filtration and drying the sample at 50° C. in the vacuum (about 200 mm) oven.

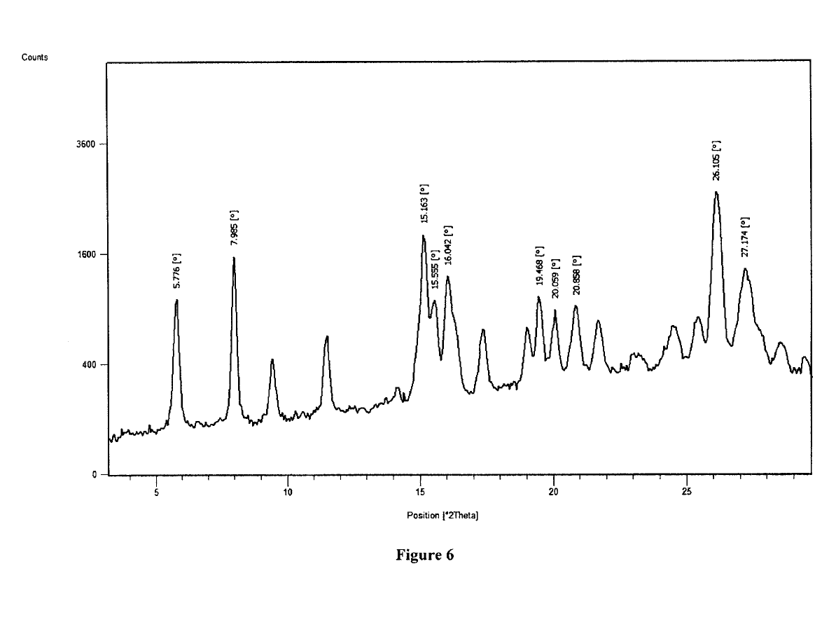

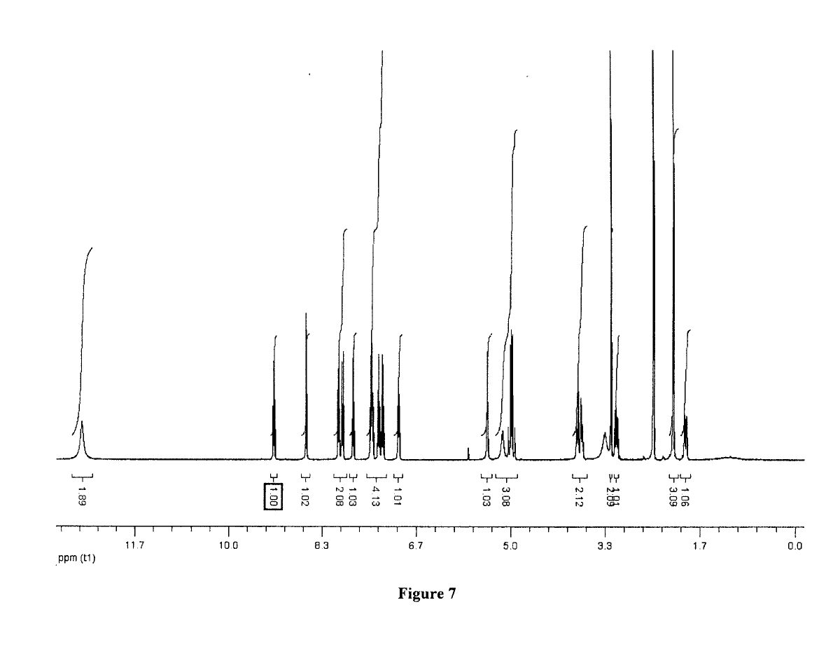

FIG. 6 shows the XRPD pattern for the lestaurtinib:malonic acid co-crystal. FIG. 7 shows the ¹H NMR spectrum for the lestaurtinib:malonic acid co-crystal. Representative XRPD peaks for the lestaurtinib:malonic acid co-crystal are listed in the following Table 3.

TABLE 3
Lestaurtinib: malonic acid XRPD peaks

| No. | Pos. [°2Th.] | d-spacing [Å] | Rel. Int. [%] |
|---|---|---|---|
| 1 | 5.78 | 15.30 | 44 |
| 2 | 7.99 | 11.07 | 70 |
| 3 | 9.42 | 9.38 | 15 |
| 4 | 11.46 | 7.72 | 22 |
| 5 | 15.16 | 5.84 | 79 |
| 6 | 15.55 | 5.70 | 36 |
| 7 | 16.04 | 5.52 | 50 |
| 8 | 17.36 | 5.11 | 21 |
| 9 | 19.01 | 4.67 | 19 |
| 10 | 19.47 | 4.56 | 33 |
| 11 | 20.06 | 4.43 | 25 |
| 12 | 20.86 | 4.26 | 27 |
| 13 | 21.71 | 4.09 | 20 |
| 14 | 23.10 | 3.85 | 5 |
| 15 | 24.53 | 3.63 | 13 |
| 16 | 25.39 | 3.51 | 15 |
| 17 | 26.11 | 3.41 | 100 |
| 18 | 27.17 | 3.28 | 43 |
| 19 | 28.45 | 3.14 | 8 |

In a preferred aspect of the present invention, representative XRPD peaks for the lestaurtinib:malonic acid co-crystal comprise one or more peaks selected from the group consisting of about 5.78, 7.99, 15.16, 15.55, 16.04, 19.47, 20.06, 20.86, 21.71, 26.11 and 27.17 degrees 2-theta. In an even more preferred aspect, representative XRPD peaks for the lestaurtinib:malonic acid co-crystal comprise one or more peaks selected from the group consisting of about 7.99, 15.16, 16.04, 26.11 and 27.17 degrees 2-theta.

### Example 3

### Lestaurtinib—Oxalic Acid Co-Crystal

100 mg of lestaurtinib was ground with 1 mole equivalent (20.5 mg) of oxalic acid in a Copley ball mill. The mixture was ground during three 30 minute periods at increasing oscillation frequencies (5 Hz, 10 Hz and 15 Hz, respectively).

8 mg of the ground sample was weighed into a small, screw top vial. 200 μl of acetonitrile was added and the vial was subjected to heat/cool cycles between ambient and 50° C. (4 hours hot, 4 hours cool) for 24 hours.

Solid was isolated by filtration and dried under ambient conditions for 1 hour before analysis.

In an alternative procedure, 80 mg of lestaurtinib with 1 mole equivalent (16.4 mg) of oxalic acid was slurried in 1.8 mL of acetonitrile. The samples were subjected to 6 cycles of maturation. Each cycle consisted of: warming over 1 hour to 50° C., holding at 50° C. for 4 hours, cooling over 3 hours (0.25° C./min) to 5° C., and holding at 5° C. for 4 hours. Solid was isolated by suction filtration and drying the sample at 50° C. in the vacuum (about 200 mm) oven.

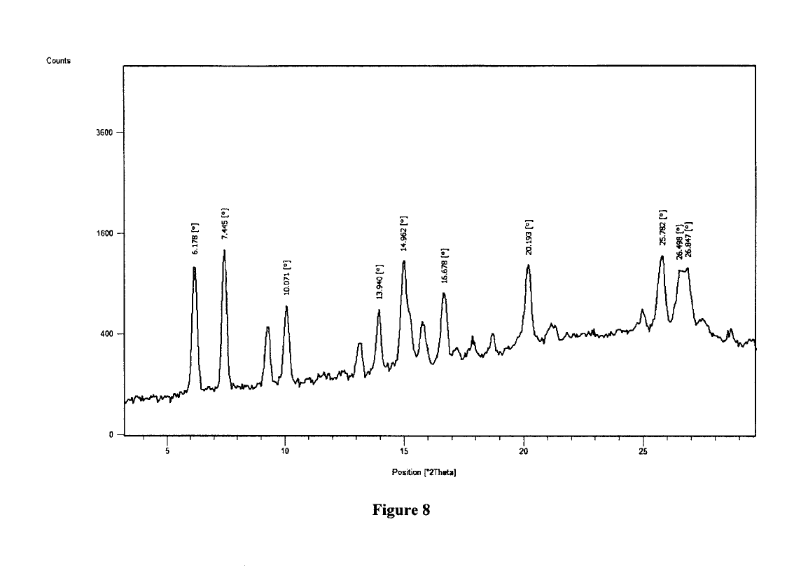

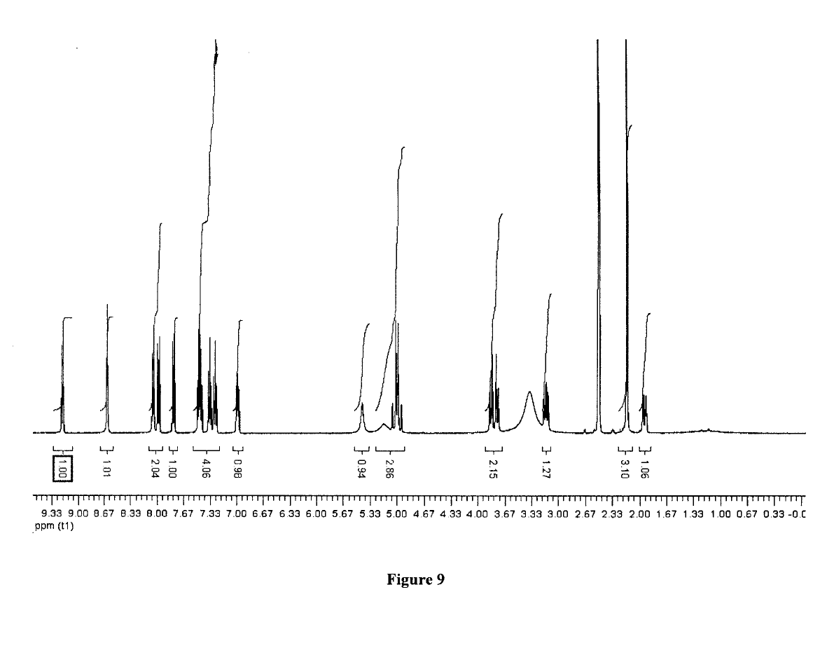

FIG. 8 shows the XRPD pattern for the lestaurtinib:oxalic acid co-crystal. FIG. 9 shows the ¹H NMR spectrum for the lestaurtinib:oxalic acid co-crystal. Representative XRPD peaks for the lestaurtinib:oxalic acid co-crystal are listed in the following Table 4.

TABLE 4
Lestaurtinib: oxalic acid XRPD

| No. | Pos. [°2Th.] | d-spacing [Å] | Rel. Int. [%] |
|---|---|---|---|
| 1 | 6.18 | 14.31 | 84 |
| 2 | 7.44 | 11.87 | 100 |
| 3 | 9.27 | 9.54 | 29 |
| 4 | 10.07 | 8.78 | 44 |
| 5 | 13.15 | 6.73 | 14 |
| 6 | 13.94 | 6.35 | 36 |
| 7 | 14.96 | 5.92 | 79 |
| 8 | 15.76 | 5.62 | 24 |
| 9 | 16.68 | 5.32 | 45 |
| 10 | 17.84 | 4.97 | 8 |
| 11 | 18.71 | 4.74 | 12 |
| 12 | 20.19 | 4.40 | 64 |
| 13 | 21.19 | 4.19 | 9 |
| 14 | 24.98 | 3.56 | 12 |
| 15 | 25.78 | 3.46 | 65 |
| 16 | 26.50 | 3.36 | 50 |
| 17 | 26.85 | 3.32 | 54 |
| 18 | 28.63 | 3.12 | 8 |

In a preferred aspect of the present invention, representative XRPD peaks for the lestaurtinib:oxalic acid co-crystal comprise one or more peaks selected from the group consisting of about 6.18, 7.44, 10.07, 13.94, 14.96, 16.68, 20.19, 25.78, 26.50 and 26.85 degrees 2-theta. In an even more preferred aspect, representative XRPD peaks for the lestaurtinib:oxalic acid co-crystal comprise one or more peaks selected from the group consisting of about 6.18, 7.44, 14.96, 20.19 and 25.78 degrees 2-theta.

### Example 4

### Lestaurtinib—Glutaric Acid Co-Crystal

100 mg of lestaurtinib was ground with 1 mole equivalent (30.1 mg) of glutaric acid in a Copley ball mill. The mixture was ground during three 30 minute periods at increasing oscillation frequencies (5 Hz, 10 Hz and 15 Hz respectively).

8 mg of the ground sample was weighed into a small, screw top vial. 200 μl of toluene was added and the vial was subjected to heat/cool cycles between ambient and 50° C. (4 hours hot, 4 hours cool) for 24 hours.

Solid was isolated by filtration and dried under ambient conditions for 1 hour before analysis.

In an alternative procedure, 80 mg of lestaurtinib with 1 mole equivalent (25.2 mg) of glutaric acid was slurried in 1.8 mL of toluene. The samples were subjected to 6 cycles of maturation. Each cycle consisted of: warming over 1 hour to 50° C., holding at 50° C. for 4 hours, cooling over 3 hours (0.25° C./min) to 5° C., and holding at 5° C. for 4 hours. Solid was isolated by suction filtration and drying the sample at 50° C. in the vacuum (about 200 mm) oven.

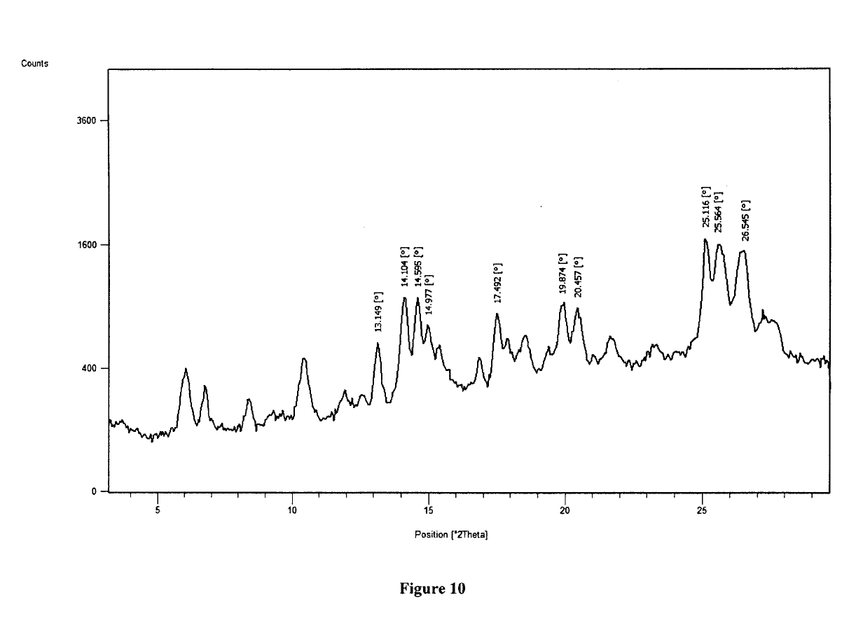

FIG. 10 shows the XRPD pattern for the lestaurtinib:glutaric acid co-crystal.

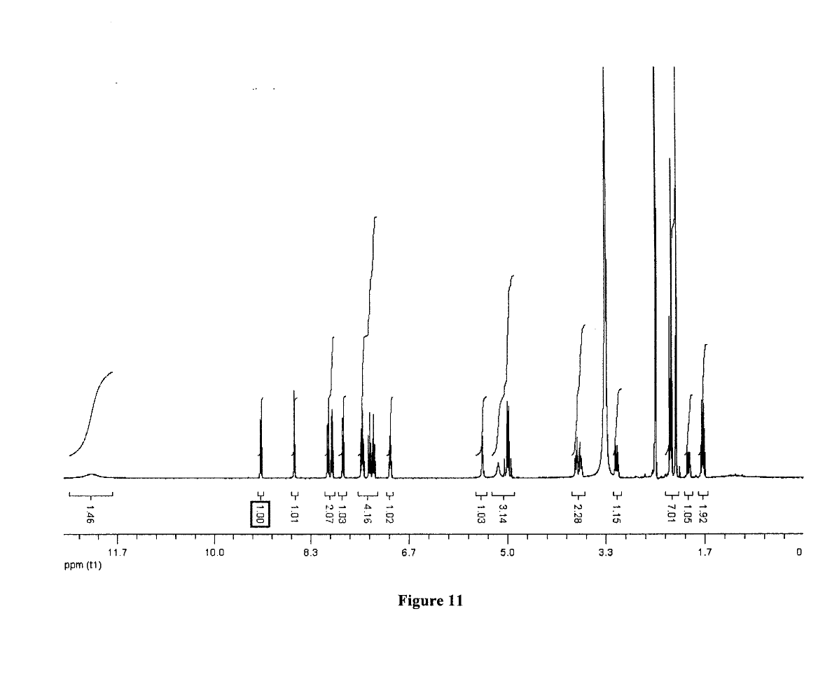

FIG. 11 shows the ¹H NMR spectrum for the lestaurtinib:glutaric acid co-crystal. Representative XRPD peaks for the lestaurtinib:glutaric acid co-crystal are listed in the following Table 5.

TABLE 5
Lestaurtinib: glutaric acid XRPD peaks

| No. | Pos. [°2Th.] | d-spacing [Å] | Rel. Int. [%] |
|---|---|---|---|
| 1 | 6.06 | 14.59 | 25 |
| 2 | 6.77 | 13.06 | 16 |
| 3 | 8.40 | 10.52 | 10 |
| 4 | 10.44 | 8.47 | 28 |
| 5 | 11.90 | 7.43 | 6 |
| 6 | 13.15 | 6.73 | 32 |
| 7 | 14.10 | 6.28 | 63 |
| 8 | 14.60 | 6.07 | 61 |
| 9 | 14.98 | 5.92 | 37 |
| 10 | 16.86 | 5.26 | 13 |
| 11 | 17.49 | 5.07 | 41 |
| 12 | 18.51 | 4.79 | 22 |
| 13 | 19.87 | 4.47 | 42 |
| 14 | 20.46 | 4.34 | 38 |
| 15 | 21.66 | 4.10 | 17 |
| 16 | 23.28 | 3.82 | 9 |
| 17 | 25.12 | 3.55 | 100 |
| 18 | 25.56 | 3.48 | 93 |
| 19 | 26.55 | 3.36 | 86 |
| 20 | 27.71 | 3.22 | 26 |

In a preferred aspect of the present invention, representative XRPD peaks for the lestaurtinib:glutaric acid co-crystal comprise one or more peaks selected from the group consisting of about 13.15, 14.10, 14.60, 14.98, 17.49, 19.87, 20.46, 25.12, 25.56 and 26.55, degrees 2-theta. In an even more preferred aspect, representative XRPD peaks for the lestaurtinib:glutaric acid co-crystal comprise one or more peaks selected from the group consisting of about 14.10, 14.60, 25.12, 25.56 and 26.55 degrees 2-theta.

### Example 5

### Lestaurtinib—Hippuric Acid Co-Crystal

100 mg of lestaurtinib was ground with 1 mole equivalent (40.8 mg) of hippuric acid in a Copley ball mill. The mixture was ground during three 30 minute periods at increasing oscillation frequencies (5 Hz, 10 Hz and 15 Hz respectively). 8 mg of the ground sample was weighed into a small, screw top vial. 200 μl of anisole was added and the vial was subjected to heat/cool cycles between ambient and 50° C. (4 hours hot, 4 hours cool) for 24 hours.

Solid was isolated by filtration and dried under ambient conditions for 1 hour before analysis.

In an alternative procedure, 80 mg of lestaurtinib with 1 mole equivalent (33.8 mg) of hippuric acid was slurried in 1.8 mL of methoxybenzene. The sample was subjected to 6 cycles of maturation. Each cycle consisted of: warming over 1 hour to 50° C., holding at 50° C. for 4 hours, cooling over 3 hours (0.25° C./min) to 5° C., and holding at 5° C. for 4 hours. Solid was isolated by suction filtration and drying the sample at 50° C. in the vacuum (about 200 mm) oven.

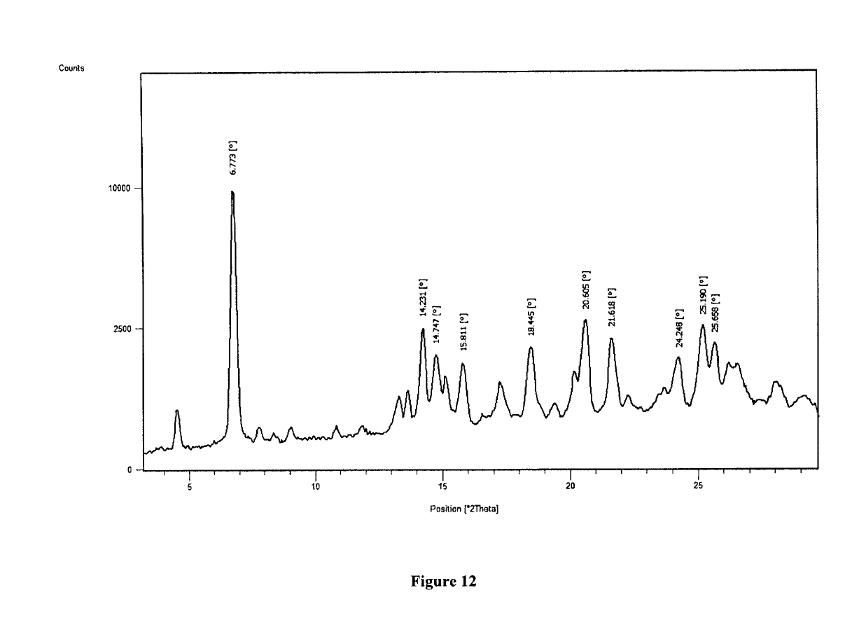

FIG. 12 shows the XRPD pattern for the lestaurtinib:hippuric acid co-crystal.

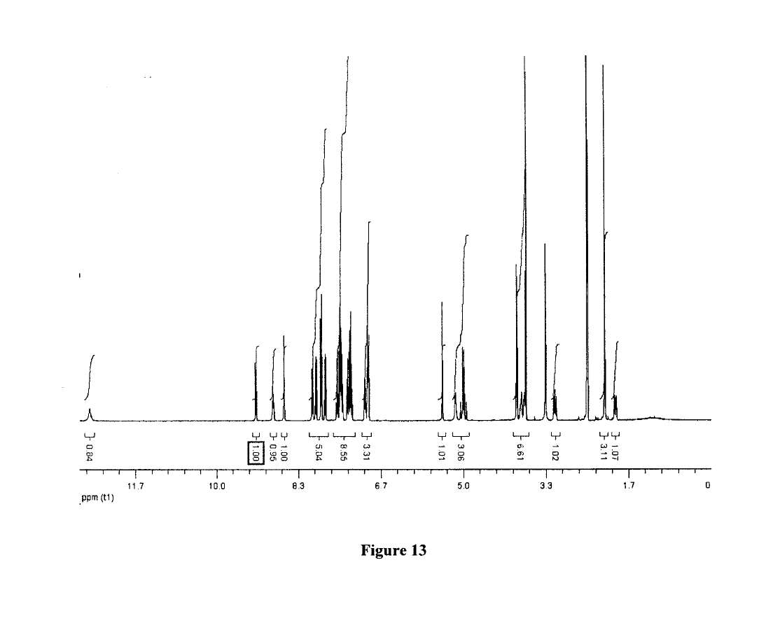

FIG. 13 shows the ¹H NMR spectrum for the lestaurtinib:hippuric acid co-crystal. Representative XRPD peaks for the lestaurtinib:hippuric acid co-crystal are listed in the following Table 6.

TABLE 6
Lestaurtinib: hippuric acid XRPD

| No. | Pos. [°2Th.] | d-spacing [Å] | Rel. Int. [%] |
|---|---|---|---|
| 1 | 6.77 | 13.05 | 100 |
| 2 | 14.23 | 6.22 | 21 |
| 3 | 14.75 | 6.01 | 12 |
| 4 | 15.15 | 5.85 | 7 |
| 5 | 15.81 | 5.61 | 11 |
| 6 | 17.25 | 5.14 | 6 |
| 7 | 18.44 | 4.81 | 15 |
| 8 | 20.14 | 4.41 | 8 |
| 9 | 20.61 | 4.31 | 24 |
| 10 | 21.62 | 4.11 | 17 |
| 11 | 24.25 | 3.67 | 10 |
| 12 | 25.19 | 3.54 | 20 |
| 13 | 25.66 | 3.47 | 13 |
| 14 | 26.17 | 3.41 | 7 |
| 15 | 26.56 | 3.36 | 7 |

In a preferred aspect of the present invention, representative XRPD peaks for the lestaurtinib:hippuric acid co-crystal comprise one or more peaks selected from the group consisting of about 6.77, 14.23, 18.44, 20.61 and 25.19 degrees 2-theta.

### Example 6

Lestaurtinib—urea co-crystal 100 mg of lestaurtinib was ground with 1 mole equivalent (13.7 mg) of urea in a Copley ball mill. The mixture was ground during three 30 minute periods at increasing oscillation frequencies (5 Hz, 10 Hz and 15 Hz respectively).

8 mg of the ground sample was weighed into a small, screw top vial. 200 μl of tert-butylmethyl ether (TBME) was added and the vial was subjected to heat/cool cycles between ambient and 50° C. (4 hours hot, 4 hours cool) for 24 hours.

Solid was isolated by filtration and dried under ambient conditions for 1 hour before analysis.

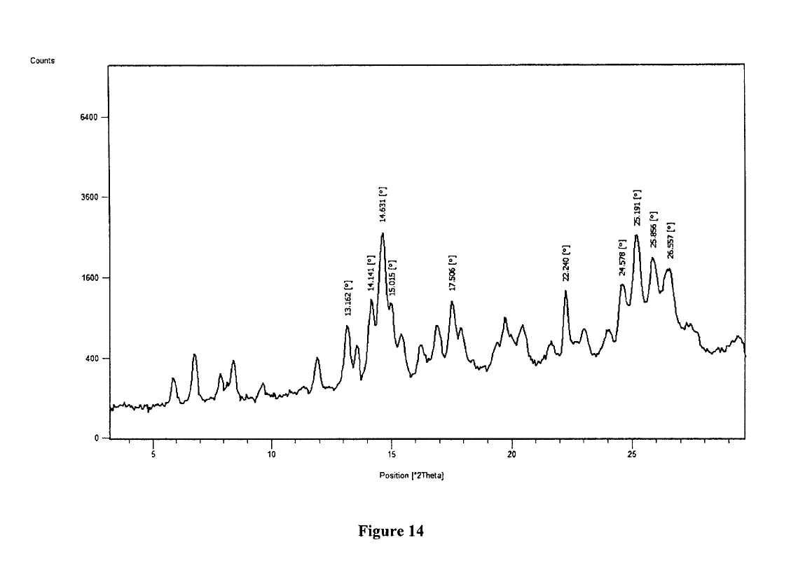

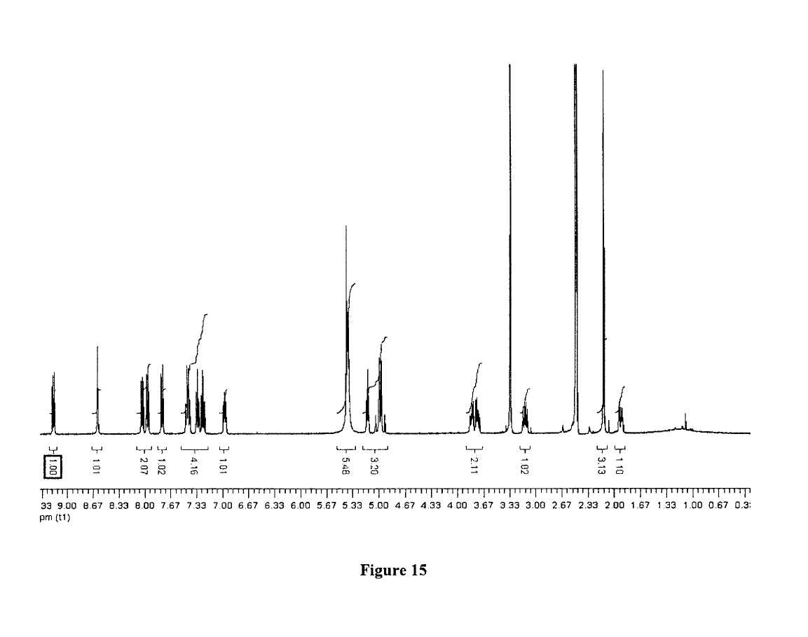

FIG. 14 shows the XRPD pattern for the lestaurtinib:urea co-crystal. FIG. 15 shows the ¹H NMR spectrum for the lestaurtinib:urea co-crystal. Representative XRPD peaks for the lestaurtinib:urea co-crystal are listed in the following Table 7.

TABLE 7
Lestaurtinib:urea XRPD peaks

| No. | Pos. [°2Th.] | d-spacing [Å] | Rel. Int. [%] |
|---|---|---|---|
| 1 | 5.88 | 15.03 | 6 |
| 2 | 6.78 | 13.03 | 15 |
| 3 | 7.85 | 11.26 | 7 |
| 4 | 8.40 | 10.52 | 12 |
| 5 | 11.91 | 7.43 | 11 |
| 6 | 13.16 | 6.73 | 25 |
| 7 | 13.57 | 6.53 | 13 |
| 8 | 14.14 | 6.26 | 39 |
| 9 | 14.63 | 6.05 | 100 |
| 10 | 15.02 | 5.90 | 35 |
| 11 | 15.43 | 5.74 | 17 |
| 12 | 16.20 | 5.47 | 11 |
| 13 | 16.90 | 5.25 | 20 |
| 14 | 17.51 | 5.07 | 36 |
| 15 | 17.91 | 4.95 | 17 |
| 16 | 19.72 | 4.50 | 24 |
| 17 | 20.48 | 4.34 | 16 |
| 18 | 21.65 | 4.10 | 8 |
| 19 | 22.24 | 4.00 | 39 |
| 20 | 23.02 | 3.86 | 13 |
| 21 | 24.04 | 3.70 | 10 |
| 22 | 24.58 | 3.62 | 38 |
| 23 | 25.19 | 3.54 | 83 |
| 24 | 25.86 | 3.45 | 61 |
| 25 | 26.56 | 3.36 | 51 |
| 26 | 27.47 | 3.25 | 10 |

In a preferred aspect of the present invention, representative XRPD peaks for the lestaurtinib:urea co-crystal comprise one or more peaks selected from the group consisting of about 14.14, 14.63, 15.02, 17.51, 19.72, 22.24, 24.58, 25.19, 25.86 and 26.56 degrees 2-theta. In an even more preferred aspect, representative XRPD peaks for the lestaurtinib:urea co-crystal comprise one or more peaks selected from the group consisting of about 14.63, 22.24, 25.19, 25.86 and 26.56 degrees 2-theta.

### Example 7

### Lestaurtinib Crystalline Form VI (1:1 Methanol Solvate)

196.8 mg of lestaurtinib was warmed with stirring in 10.0 mL of anhydrous methanol to the boiling point and the saturated solution heated for an additional 2-3 minutes. The saturated solution was syringe filtered into a clean, pre-warmed vial and solution cooled initially at room temperature and then stored at 4-8° C. overnight. The solid was isolated by decantation and solid allowed to dry before analysis.

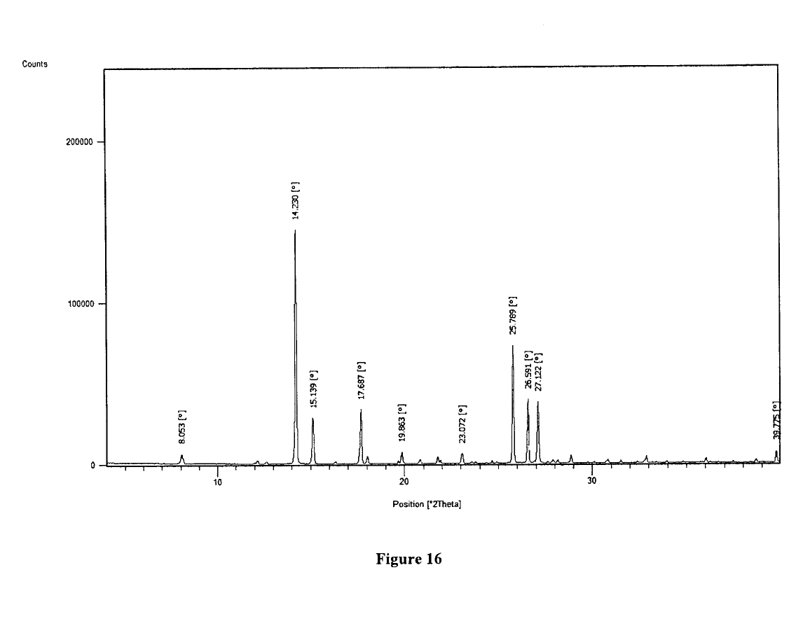

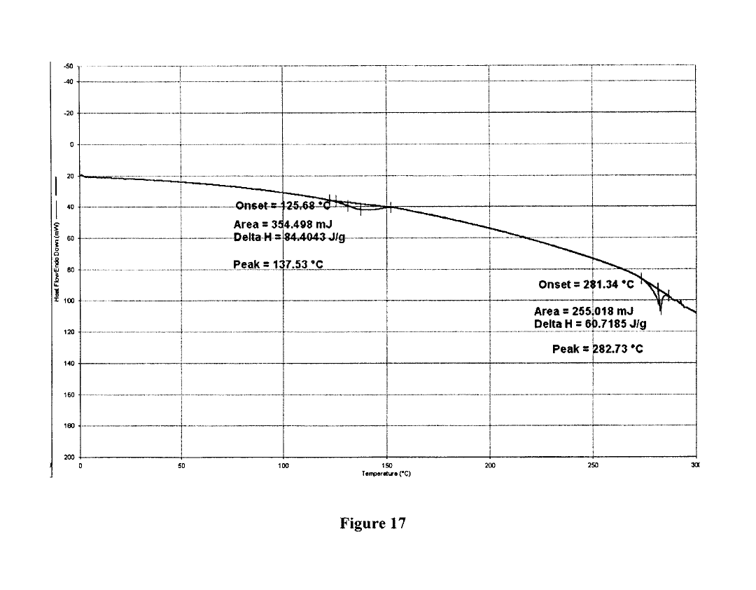

FIG. 16 shows the XRPD pattern for the Lestaurtinib Crystalline Form VI. FIG. 17 shows the DSC/TGA overlay of the Lestaurtinib Crystalline Form VI. Representative XRPD peaks for the Lestaurtinib Crystalline Form VI are listed in the following Table 8.

TABLE 8
Lestaurtinib Crystalline Form VI XRPD peaks

| No. | Pos. [°2Th.] | d-spacing [Å] | Rel. Int. [%] |
|---|---|---|---|
| 1 | 8.05 | 10.97 | 4 |
| 2 | 14.23 | 6.22 | 100 |
| 3 | 15.14 | 5.85 | 19 |
| 4 | 17.69 | 5.01 | 23 |
| 5 | 18.04 | 4.91 | 3 |
| 6 | 19.86 | 4.47 | 5 |
| 7 | 23.07 | 3.85 | 4 |
| 8 | 25.79 | 3.45 | 50 |
| 9 | 26.59 | 3.35 | 27 |
| 10 | 27.12 | 3.29 | 26 |
| 11 | 28.88 | 3.09 | 3 |
| 12 | 39.77 | 2.26 | 5 |

In a preferred aspect of the present invention, representative XRPD peaks for Lestaurtinib Crystalline Form VI comprise one or more peaks selected from the group consisting of about 8.05, 14.23, 15.14, 17.69, 19.86, 23.07, 25.79, 26.59, 27.12 and 39.77 degrees 2-theta. In an even more preferred aspect, representative XRPD peaks for Lestaurtinib Crystalline Form VI comprise one or more peaks selected from the group consisting of about 14.23, 17.69, 25.79, 26.59 and 27.12 degrees 2-theta.

### Example 8

### Lestaurtinib Crystalline Form VII (Propionitrile/Water Solvate)

441.9 mg of lestaurtinib was warmed with stirring in 50 mL of propionitrile to the boiling point and the saturated solution heated an additional 2-3 minutes. The saturated solution was syringe filtered and the clear solution was concentrated with heating and stirring to 5-10 mL total volume. The concentrated solution was allowed to cool at 4-8° C. over 5 days.

The solid was isolated by decantation and allowed to dry on absorbant paper before analysis.

In an alternative procedure, 40 mg of amorphous form of lestaurtinib in 400 μL of solvent were slurried in formamide. These mixtures were slurried for 48 hours with alternating 4 hour periods at 50° C. and 5° C. (−0.5° C./min). The solid was isolated by filtration. The material was dried at 40° C. under house vacuum during 1 hour.

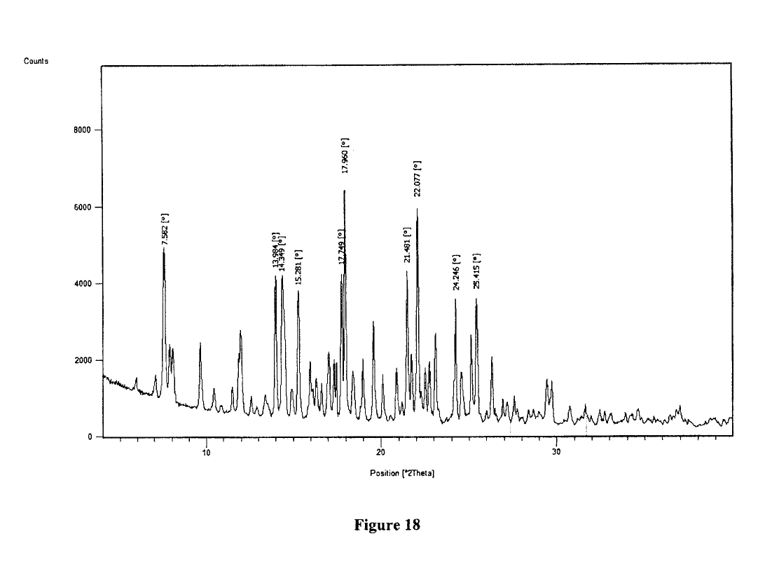

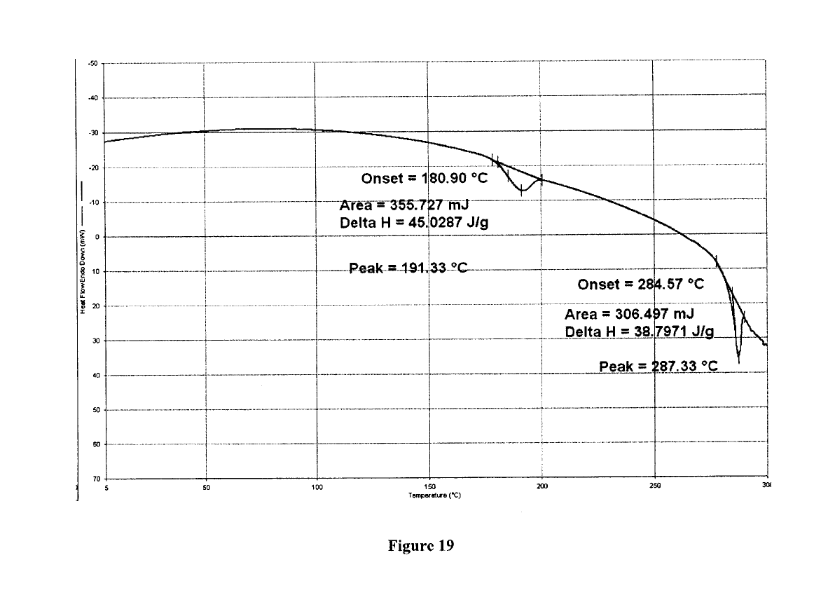

FIG. 18 shows the XRPD pattern for Lestaurtinib Crystalline Form VII. FIG. 19 shows the DSC/TGA overlay of Lestaurtinib Crystalline Form VII. Representative XRPD peaks for Lestaurtinib Crystalline Form VII are listed in the following Table 9.

TABLE 9
Lestaurtinib Crystalline Form VII XRPD peaks

| No. | Pos. [°2Th.] | d-spacing [Å] | Rel. Int. [%] |
|---|---|---|---|
| 1 | 7.58 | 11.65 | 62 |
| 2 | 7.98 | 11.06 | 17 |
| 3 | 9.65 | 9.15 | 29 |
| 4 | 11.49 | 7.69 | 11 |
| 5 | 12.02 | 7.36 | 33 |
| 6 | 13.98 | 6.33 | 59 |
| 7 | 14.35 | 6.17 | 58 |
| 8 | 14.92 | 5.93 | 11 |
| 9 | 15.28 | 5.79 | 52 |
| 10 | 16.30 | 5.43 | 16 |
| 11 | 17.04 | 5.20 | 26 |
| 12 | 17.33 | 5.11 | 24 |
| 13 | 17.75 | 4.99 | 61 |
| 14 | 17.96 | 4.94 | 100 |
| 15 | 18.42 | 4.81 | 19 |
| 16 | 18.97 | 4.67 | 26 |
| 17 | 19.56 | 4.53 | 42 |
| 18 | 20.12 | 4.41 | 15 |
| 19 | 20.85 | 4.26 | 21 |
| 20 | 21.48 | 4.13 | 63 |
| 21 | 21.71 | 4.09 | 28 |
| 22 | 22.08 | 4.02 | 91 |
| 23 | 22.72 | 3.91 | 24 |
| 24 | 23.08 | 3.85 | 38 |
| 25 | 24.25 | 3.67 | 52 |
| 26 | 25.12 | 3.54 | 36 |
| 27 | 25.42 | 3.50 | 51 |
| 28 | 26.30 | 3.39 | 27 |
| 29 | 29.44 | 3.03 | 10 |

In a preferred aspect of the present invention, representative XRPD peaks for the Lestaurtinib:propionitrile solvate comprise one or more peaks selected from the group consisting of about 7.58, 13.98, 14.35, 15.28, 17.75, 17.96, 21.48, 22.08, 24.25 and 25.42 degrees 2-theta. In an even more preferred aspect, representative XRPD peaks for the Lestaurtinib:propionitrile solvate comprise one or more peaks selected from the group consisting of about 7.58, 17.75, 17.96, 21.48 and 22.08 degrees 2-theta.

### Example 9

### Lestaurtinib Crystalline Form VIII (Acetone/Water Solvate)

1.0 g of lestaurtinib was warmed with stirring in 90 mL of acetone to the boiling point and heated an additional 2-3 minutes. The saturated solution was suction filtered and the clear, yellow solution was concentrated to a volume of approximately 40 mL. The solution was cooled at 4-8° C. over 3 days.

In an alternative procedure, 40 mg of amorphous form of lestaurtinib in 400 μL of solvent were slurried in formamide. These mixtures were slurried for 48 hours with alternating 4 hour periods at 50° C. and 5° C. (−0.5° C./min). The solid was isolated by filtration. The material was dried at 40° C. under house vacuum during 1 hour.

The solid material was isolated by decantation and solid allowed to dry on absorbant paper before analysis.

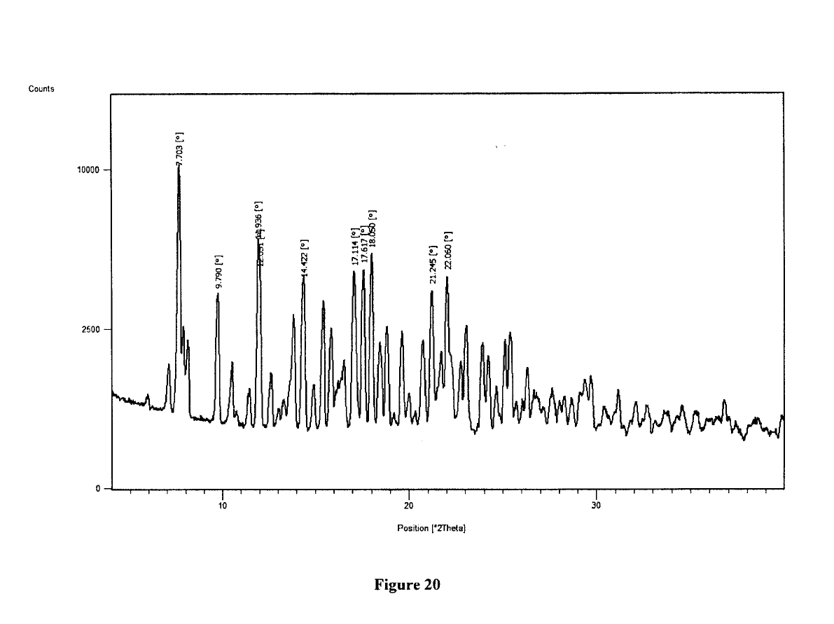

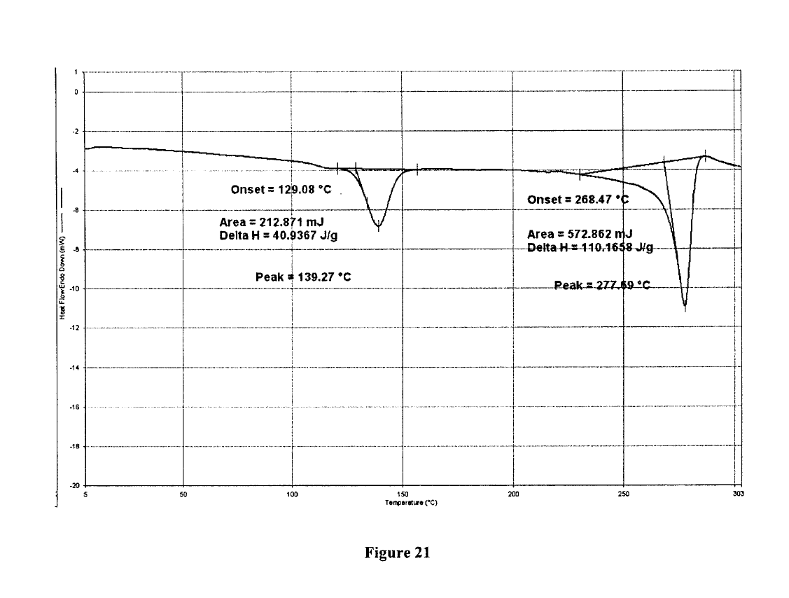

FIG. 20 shows the XRPD pattern for Lestaurtinib Crystalline Form VIII. FIG. 21 shows the DSC/TGA overlay of Lestaurtinib Crystalline Form VIII. Representative XRPD peaks for Lestaurtinib Crystalline Form VIII are listed in the following Table 10.

TABLE 10
Lestaurtinib Crystalline Form VIII. XRPD peaks

| No. | Pos. [°2Th.] | d-spacing [Å] | Rel. Int. [%] |
|---|---|---|---|
| 1 | 7.70 | 11.47 | 100 |
| 2 | 7.91 | 11.17 | 22 |
| 3 | 8.15 | 10.84 | 18 |
| 4 | 9.79 | 9.03 | 34 |
| 5 | 10.53 | 8.39 | 13 |
| 6 | 11.94 | 7.41 | 58 |
| 7 | 12.05 | 7.34 | 44 |
| 8 | 13.88 | 6.38 | 25 |
| 9 | 14.42 | 6.14 | 40 |
| 10 | 15.47 | 5.72 | 31 |
| 11 | 15.88 | 5.58 | 21 |
| 12 | 17.11 | 5.18 | 44 |
| 13 | 17.49 | 5.07 | 29 |
| 14 | 17.62 | 5.03 | 45 |
| 15 | 17.92 | 4.95 | 27 |
| 16 | 18.05 | 4.91 | 52 |
| 17 | 18.46 | 4.80 | 18 |
| 18 | 18.86 | 4.70 | 23 |
| 19 | 19.66 | 4.51 | 21 |
| 20 | 20.77 | 4.27 | 18 |
| 21 | 21.24 | 4.18 | 36 |
| 22 | 21.73 | 4.09 | 15 |
| 23 | 21.96 | 4.04 | 29 |
| 24 | 22.06 | 4.03 | 42 |
| 25 | 23.08 | 3.85 | 24 |
| 26 | 23.96 | 3.71 | 18 |
| 27 | 24.26 | 3.67 | 14 |
| 28 | 25.15 | 3.54 | 19 |
| 29 | 25.34 | 3.51 | 18 |
| 30 | 25.47 | 3.49 | 18 |

In a preferred aspect of the present invention, representative XRPD peaks for Lestaurtinib Crystalline Form VIII comprise one or more peaks selected from the group consisting of about 7.70, 9.79, 11.94, 12.05, 14.42, 17.11, 17.62, 18.05, 21.24 and 22.06 degrees 2-theta. In an even more preferred aspect, representative XRPD peaks for Lestaurtinib Crystalline Form VIII comprise one or more peaks selected from the group consisting of about 7.70, 11.94, 12.05, 17.11, 17.62 and 18.05 degrees 2-theta.

### Example 10

### Lestaurtinib Crystalline Form IX (2-butanone Solvate)

1.0 g of lestaurtinib was warmed with stirring to the boiling point in 100 mL of 2-butanone. The saturated solution was syringe filtered and the resulting clear, yellow solution was concentrated to a volume of approximately 20 mL. The concentrated solution was cooled in the freezer overnight.

The solid was isolated by decantation and the sticky solid allowed to dry on absorbant paper. When dry, the solid was ground to a powder using a mortar and pestle.

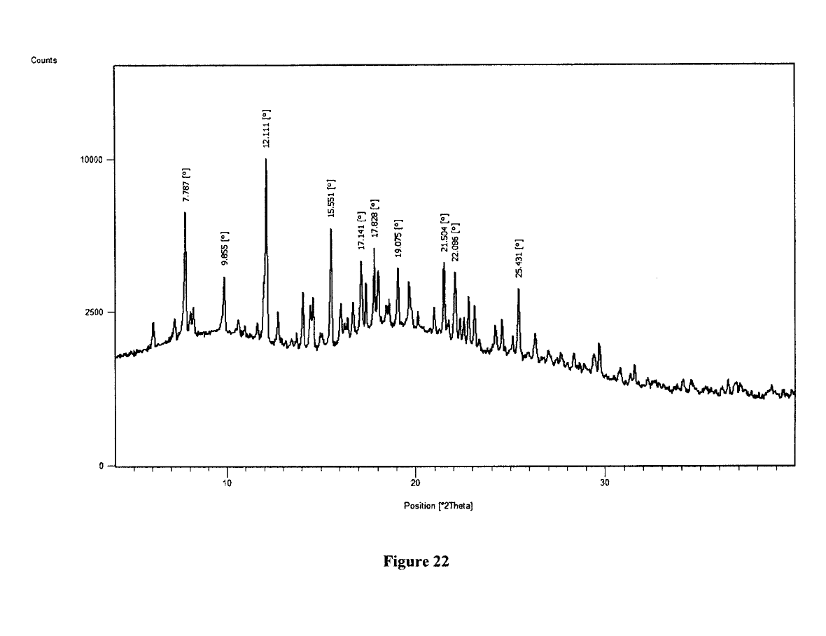

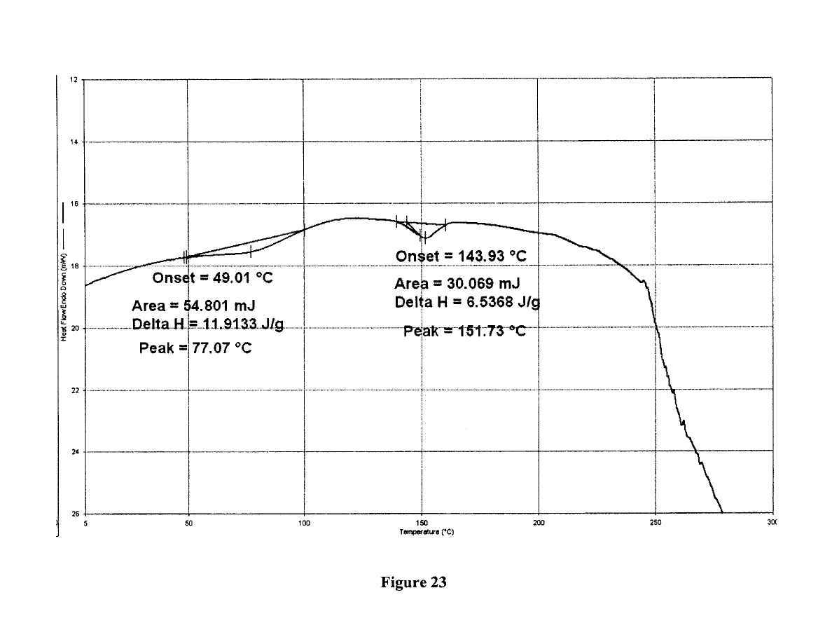

FIG. 22 shows the XRPD pattern for Lestaurtinib Crystalline Form IX. FIG. 23 shows the DSC/TGA overlay of Lestaurtinib Crystalline Form IX. Representative XRPD peaks for Lestaurtinib Crystalline Form IX are listed in the following Table 11.

TABLE 11
Lestaurtinib Crystalline Form IX XRPD peaks

| No. | Pos. [°2Th.] | d-spacing [Å] | Rel. Int. [%] |
|---|---|---|---|
| 1 | 6.06 | 14.57 | 8 |
| 2 | 7.79 | 11.34 | 60 |
| 3 | 8.05 | 10.98 | 9 |
| 4 | 8.20 | 10.77 | 10 |
| 5 | 9.85 | 8.97 | 23 |
| 6 | 12.11 | 7.30 | 100 |
| 7 | 12.72 | 6.95 | 11 |
| 8 | 14.06 | 6.30 | 20 |
| 9 | 14.45 | 6.12 | 15 |
| 10 | 14.60 | 6.06 | 18 |
| 11 | 15.55 | 5.69 | 53 |
| 12 | 16.07 | 5.51 | 13 |
| 13 | 16.71 | 5.30 | 12 |
| 14 | 17.14 | 5.17 | 30 |
| 15 | 17.40 | 5.09 | 19 |
| 16 | 17.83 | 4.97 | 35 |
| 17 | 18.04 | 4.91 | 23 |
| 18 | 18.60 | 4.77 | 10 |
| 19 | 19.07 | 4.65 | 24 |
| 20 | 19.67 | 4.51 | 18 |
| 21 | 20.97 | 4.23 | 9 |
| 22 | 21.50 | 4.13 | 31 |
| 23 | 22.09 | 4.02 | 27 |
| 24 | 22.34 | 3.98 | 8 |
| 25 | 22.55 | 3.94 | 9 |
| 26 | 22.79 | 3.90 | 17 |
| 27 | 23.10 | 3.85 | 15 |
| 28 | 24.56 | 3.62 | 10 |
| 29 | 25.43 | 3.50 | 24 |
| 30 | 29.68 | 3.01 | 9 |

In a preferred aspect of the present invention, representative XRPD peaks for Lestaurtinib Crystalline Form IX comprise one or more peaks selected from the group consisting of about 7.79, 9.85, 12.11, 15.55, 17.14, 17.83, 19.07, 21.50, 22.09 and 25.43 degrees 2-theta. In an even more preferred aspect, representative XRPD peaks for Lestaurtinib Crystalline Form IX comprise one or more peaks selected from the group consisting of about 7.79, 12.11, 15.55, 17.83 and 21.50 degrees 2-theta.

### Example 11

### Lestaurtinib Crystalline Form X (Tetrahydrofuran/Methanol (5:1) Solvate)

560.0 mg of lestaurtinib was warmed with stirring to the boiling point in 15.5 mL of 5:1 (v:v) tetrahydrofuran-methanol and syringe-filtered. The solution was concentrated to dryness and redissolved in approximately 4 mL of freshly prepared 5:1 tetrahydrofuran-methanol.

The solution was cooled in the freezer over 6 days, and decantation produced a waxy syrup that, when allowed to dry, produced a glassy solid.

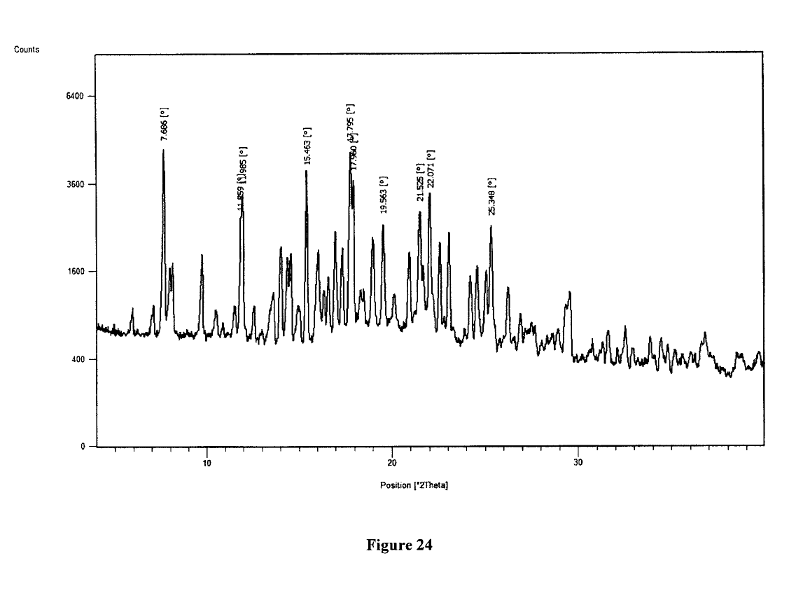

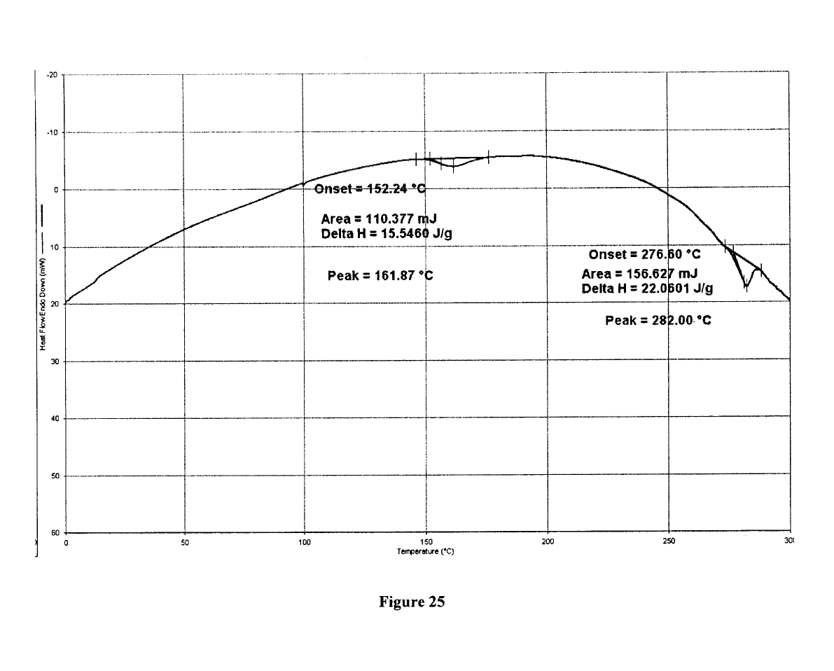

FIG. 24 shows the XRPD pattern for Lestaurtinib Crystalline Form X. FIG. 25 shows the DSC/TGA overlay of the Lestaurtinib Crystalline Form X. Representative XRPD peaks for Lestaurtinib Crystalline Form X are listed in the following Table 12.

TABLE 12
Lestaurtinib Crystalline Form X XRPD peaks

| No. | Pos. [°2Th.] | d-spacing [Å] | Rel. Int. [%] |
|---|---|---|---|
| 1 | 7.69 | 11.49 | 100 |
| 2 | 8.00 | 11.04 | 26 |
| 3 | 8.14 | 10.85 | 28 |
| 4 | 9.75 | 9.06 | 31 |
| 5 | 11.86 | 7.46 | 52 |
| 6 | 11.99 | 7.38 | 69 |
| 7 | 14.07 | 6.29 | 37 |
| 8 | 14.38 | 6.15 | 33 |
| 9 | 14.60 | 6.06 | 32 |
| 10 | 15.46 | 5.73 | 83 |
| 11 | 16.07 | 5.51 | 34 |
| 12 | 16.62 | 5.33 | 19 |
| 13 | 16.98 | 5.22 | 42 |
| 14 | 17.37 | 5.10 | 33 |
| 15 | 17.79 | 4.98 | 96 |
| 16 | 17.96 | 4.93 | 74 |
| 17 | 19.05 | 4.66 | 34 |
| 18 | 19.56 | 4.53 | 46 |
| 19 | 20.95 | 4.24 | 31 |
| 20 | 21.52 | 4.13 | 55 |
| 21 | 21.69 | 4.09 | 20 |
| 22 | 22.07 | 4.02 | 64 |
| 23 | 22.61 | 3.93 | 38 |
| 24 | 23.08 | 3.85 | 43 |
| 25 | 24.20 | 3.68 | 23 |
| 26 | 24.58 | 3.62 | 29 |
| 27 | 25.05 | 3.55 | 27 |
| 28 | 25.35 | 3.51 | 51 |
| 29 | 26.27 | 3.39 | 19 |
| 30 | 29.59 | 3.02 | 19 |

In a preferred aspect of the present invention, representative XRPD peaks for Lestaurtinib Crystalline Form X comprise one or more peaks selected from the group consisting of about 7.69, 11.86, 11.99, 15.46, 17.79, 17.96, 19.56, 21.52, 22.07 and 25.35 degrees 2-theta. In an even more preferred aspect, representative XRPD peaks for Lestaurtinib Crystalline Form X comprise one or more peaks selected from the group consisting of about 7.69, 11.99, 15.46, 17.79, and 17.96 degrees 2-theta.

### Example 12

### Lestaurtinib Crystalline Form XI (1:3 Formamide Solvate)

20 mg of a ground lestaurtinib sample was weighed into a small, screw top vial. 500 μl of formamide was added and the vial was subjected to heat/cool cycles between ambient and 50° C. (4 hours hot, 4 hours cool) for 24 hours. Solid was isolated by filtration and air dried for 1 hour before analysis.

In an alternative procedure, 40 mg of amorphous form of lestaurtinib in 400 μL of solvent were slurried in formamide. These mixtures were slurried for 48 hours with alternating 4 hour periods at 50° C. and 5° C. (−0.5° C./min). The solid was isolated by filtration. The material was dried at 40° C. under house vacuum during 1 hour.

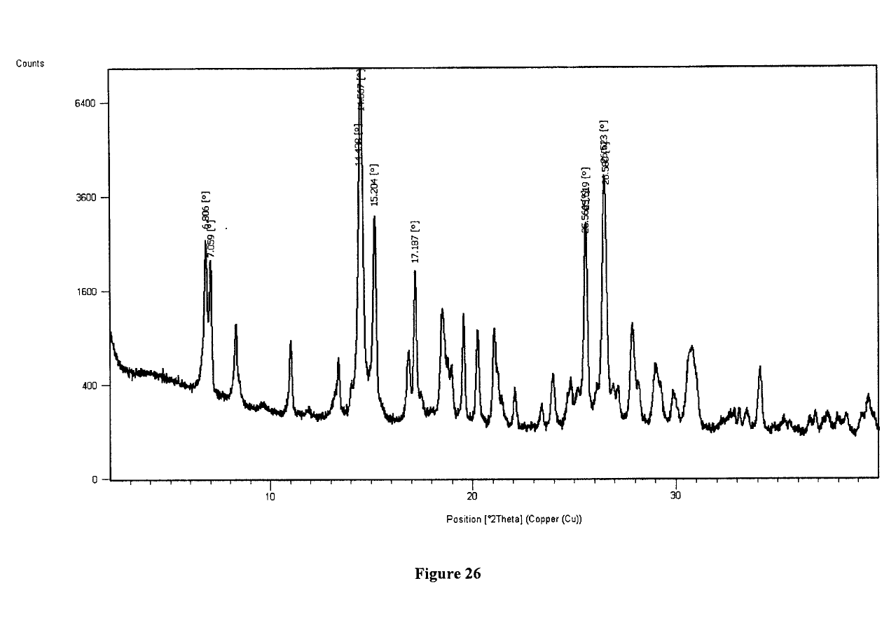

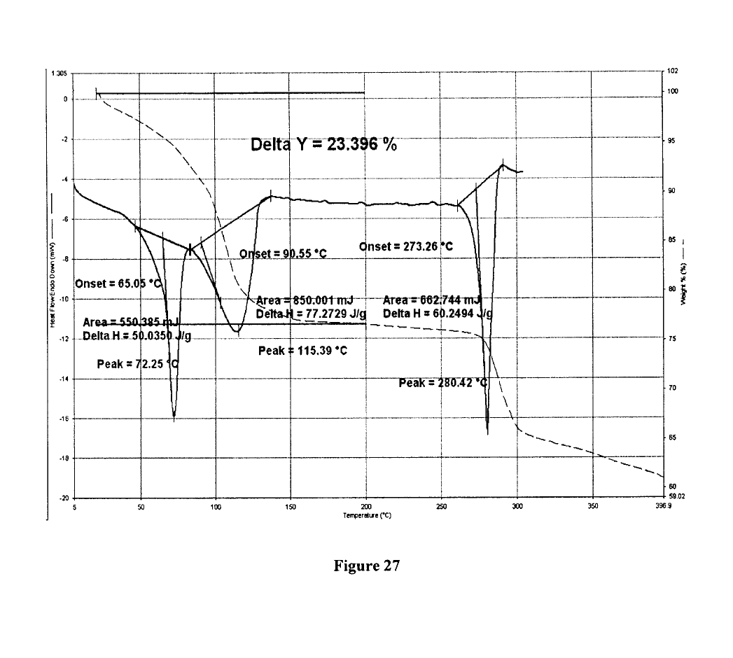

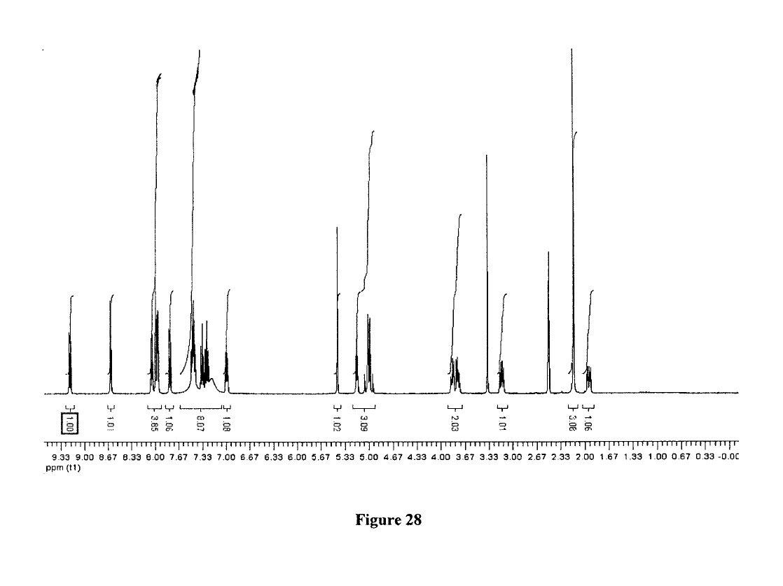

FIG. 26 shows the XRPD pattern for the Lestaurtinib Crystalline Form XI. FIG. 27 shows the DSC/TGA overlay of the Lestaurtinib Crystalline Form XI. FIG. 28 shows the ¹H NMR spectrum for the Lestaurtinib Crystalline Form XI. Representative XRPD peaks for the Lestaurtinib Crystalline Form XI are listed in the following Table 13.

TABLE 13
Lestaurtinib Crystalline Form XI XRPD peaks

| No. | Pos. [°2Th.] | d-spacing [Å] | Rel. Int. [%] |
|---|---|---|---|
| 1 | 6.71 | 13.17 | 48 |
| 2 | 8.15 | 10.84 | 5 |
| 3 | 10.94 | 8.09 | 15 |
| 4 | 13.32 | 6.65 | 8 |
| 5 | 14.44 | 6.13 | 100 |
| 6 | 15.10 | 5.87 | 34 |
| 7 | 17.11 | 5.18 | 32 |
| 8 | 18.55 | 4.78 | 34 |
| 9 | 19.54 | 4.54 | 43 |
| 10 | 20.15 | 4.41 | 6 |
| 11 | 21.18 | 4.20 | 18 |
| 12 | 22.01 | 4.04 | 11 |
| 13 | 24.83 | 3.59 | 5 |
| 14 | 25.61 | 3.48 | 61 |
| 15 | 26.51 | 3.36 | 97 |
| 16 | 27.80 | 3.21 | 58 |
| 17 | 29.12 | 3.07 | 16 |

In a preferred aspect of the present invention, representative XRPD peaks for the Lestaurtinib Crystalline Form XI comprise one or more peaks selected from the group consisting of about 6.71, 14.44, 15.10, 17.11, 18.55, 19.54, 21.18, 25.61, 26.51 and 27.80 degrees 2-theta. In an even more preferred aspect, representative XRPD peaks for the Lestaurtinib Crystalline Form XI comprise one or more peaks selected from the group consisting of about 6.71, 14.44, 25.61, 26.51 and 27.80 degrees 2-theta.

### Example 13

### Lestaurtinib Crystalline Form XII (Chlorobenzene Solvate)

40 mg of amorphous form of lestaurtinib in 400 μL of solvent were slurried in chlorobenzene. These mixtures were slurried for 48 hours with alternating 4 hour periods at 50° C. and 5° C. (−0.5° C./min). The solid was isolated by filtration. The material was dried at 40° C. under house vacuum for 1 hour.

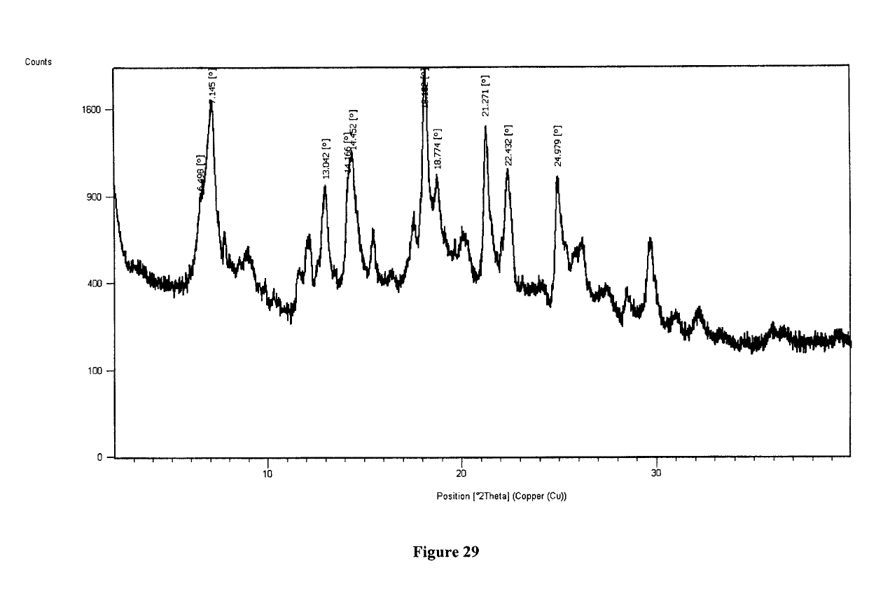

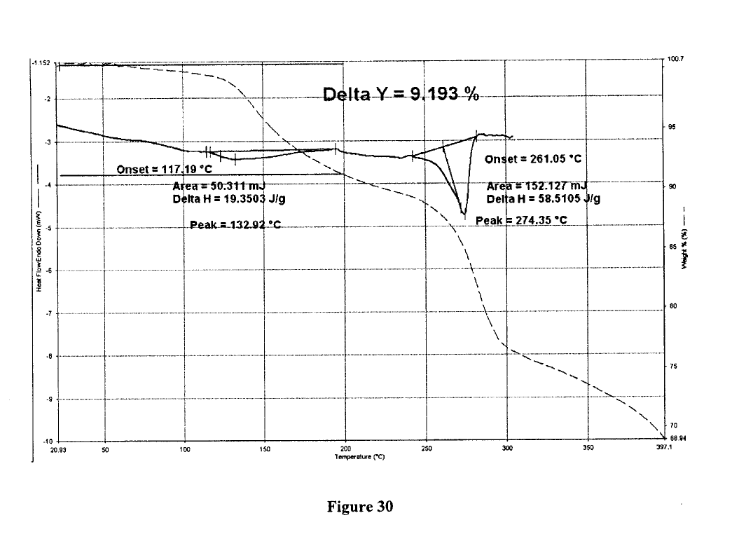

FIG. 29 shows the XRPD pattern for the lestaurtinib Crystalline Form XII. FIG. 30 shows the DSC/TGA overlay of the Lestaurtinib Crystalline Form XII. Representative XRPD peaks for the Lestaurtinib Crystalline Form XII are listed in the following Table 14.

TABLE 14
Lestaurtinib Crystalline Form XII XRPD peaks

| No. | Pos. [°2Th.] | d-spacing [Å] | Rel. Int. [%] |
|---|---|---|---|
| 1 | 6.50 | 13.59 | 29 |
| 2 | 7.15 | 12.36 | 74 |
| 3 | 7.76 | 11.38 | 18 |
| 4 | 8.94 | 9.89 | 13 |
| 5 | 11.55 | 7.65 | 9 |
| 6 | 12.04 | 7.35 | 18 |
| 7 | 12.22 | 7.24 | 20 |
| 8 | 13.04 | 6.78 | 39 |
| 9 | 14.17 | 6.25 | 41 |
| 10 | 14.45 | 6.12 | 51 |
| 11 | 15.48 | 5.72 | 24 |
| 12 | 16.37 | 5.41 | 11 |
| 13 | 17.58 | 5.04 | 29 |
| 14 | 18.18 | 4.88 | 100 |
| 15 | 18.77 | 4.72 | 44 |
| 16 | 20.05 | 4.42 | 24 |
| 17 | 21.27 | 4.17 | 70 |
| 18 | 22.43 | 3.96 | 47 |
| 19 | 24.22 | 3.67 | 9 |
| 20 | 24.98 | 3.56 | 47 |
| 21 | 26.22 | 3.40 | 22 |
| 22 | 27.49 | 3.24 | 10 |
| 23 | 28.49 | 3.13 | 9 |
| 24 | 29.76 | 3.00 | 23 |
| 25 | 32.20 | 2.78 | 5 |

In a preferred aspect of the present invention, representative XRPD peaks for the Lestaurtinib Crystalline Form XII comprise one or more peaks selected from the group consisting of about 6.50, 7.15, 13.04, 14.17, 14.45, 18.18, 18.77, 21.27, 22.43 and 24.98 degrees 2-theta. In an even more preferred aspect, representative XRPD peaks for the Lestaurtinib Crystalline Form XII comprise one or more peaks selected from the group consisting of about 7.15, 18.18, 18.77, 21.27, and 24.98 degrees 2-theta.

### Example 14

### Lestaurtinib Crystalline Form XIII (Hemihydrate)

40 mg of amorphous form of lestaurtinib was slurried in water (10 volumes (40 mg in 400 μL)). The sample was heated from 20° C. to 80° C. at a rate of 4.8° C./min and after 30 minutes cooled at a slow rate (0.25° C./min) to a final temperature of 5° C. and kept at that temperature for 18 h. The solid was isolated by filtration. The material was dried at 40° C. under house vacuum for 1 hour.

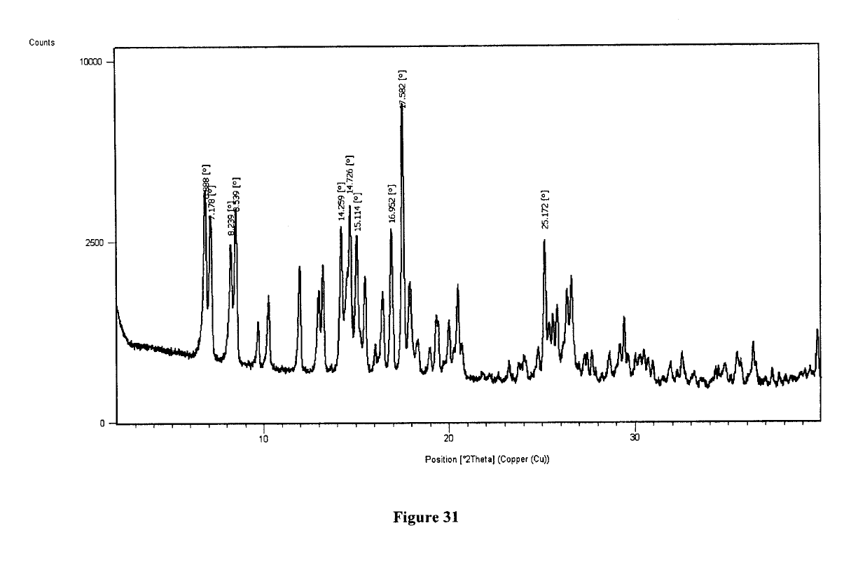

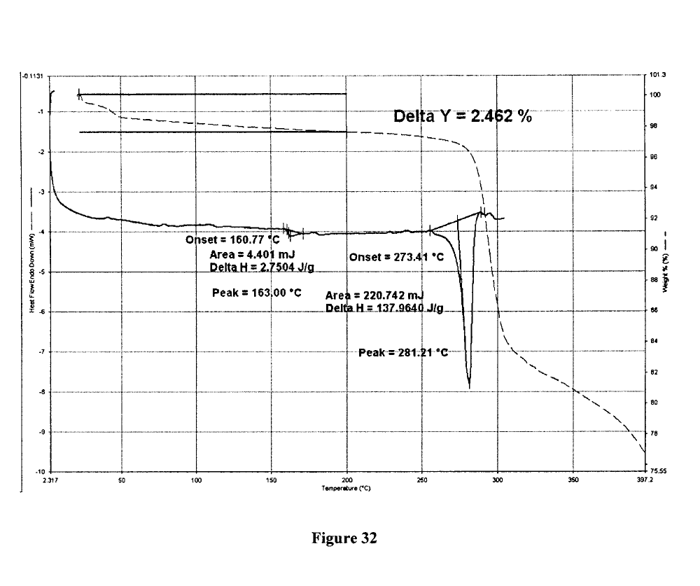

FIG. 31 shows the XRPD pattern for the Lestaurtinib Crystalline Form XIII. FIG. 32 shows the DSC/TGA overlay of the Lestaurtinib Crystalline Form XIII. Representative XRPD peaks for the Lestaurtinib Crystalline Form XIII are listed in the following Table 15.

TABLE 15
Lestaurtinib Crystalline Form XIII XRPD peaks

| No. | Pos. [°2Th.] | d-spacing [Å] | Rel. Int. [%] |
|---|---|---|---|
| 1 | 6.89 | 12.82 | 46 |
| 2 | 7.18 | 12.31 | 37 |
| 3 | 8.24 | 10.72 | 31 |
| 4 | 8.54 | 10.35 | 41 |
| 5 | 9.70 | 9.11 | 8 |
| 6 | 10.30 | 8.58 | 13 |
| 7 | 12.01 | 7.37 | 22 |
| 8 | 13.01 | 6.80 | 17 |
| 9 | 13.27 | 6.67 | 21 |
| 10 | 14.26 | 6.21 | 38 |
| 11 | 14.73 | 6.01 | 50 |
| 12 | 15.11 | 5.86 | 33 |
| 13 | 15.50 | 5.71 | 20 |
| 14 | 15.54 | 5.70 | 20 |
| 15 | 16.45 | 5.38 | 16 |
| 16 | 16.95 | 5.23 | 37 |
| 17 | 17.58 | 5.04 | 100 |
| 18 | 17.95 | 4.94 | 18 |
| 19 | 19.32 | 4.59 | 10 |
| 20 | 19.46 | 4.56 | 7 |
| 21 | 20.00 | 4.44 | 9 |
| 22 | 20.48 | 4.33 | 19 |
| 23 | 25.17 | 3.54 | 35 |
| 24 | 25.39 | 3.51 | 9 |
| 25 | 25.59 | 3.48 | 11 |
| 26 | 25.81 | 3.45 | 13 |
| 27 | 26.37 | 3.38 | 17 |
| 28 | 26.58 | 3.35 | 22 |
| 29 | 26.63 | 3.34 | 20 |
| 30 | 29.42 | 3.03 | 10 |

In a preferred aspect of the present invention, representative XRPD peaks for the Lestaurtinib Crystalline Form XIII comprise one or more peaks selected from the group consisting of about 6.89, 7.18, 8.24, 8.54, 14.26, 14.73, 15.11, 16.95, 17.58 and 25.17 degrees 2-theta. In an even more preferred aspect, representative XRPD peaks for the Lestaurtinib Crystalline Form XIII comprise one or more peaks selected from the group consisting of about 6.89, 14.26, 14.73, 16.95 and 17.58 degrees 2-theta.

### Example 15

### Lestaurtinib Crystalline Form XIV (1-butanol Solvate)

A solution of lestaurtinib in 1-butanol was allowed to slowly evaporate to dryness under ambient conditions. The rate of evaporation was constrained by use of air tight film covers containing small holes

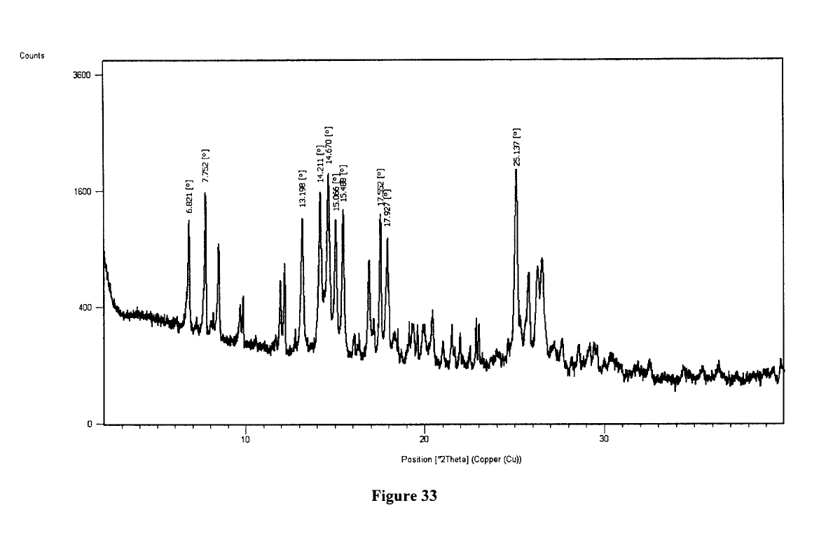

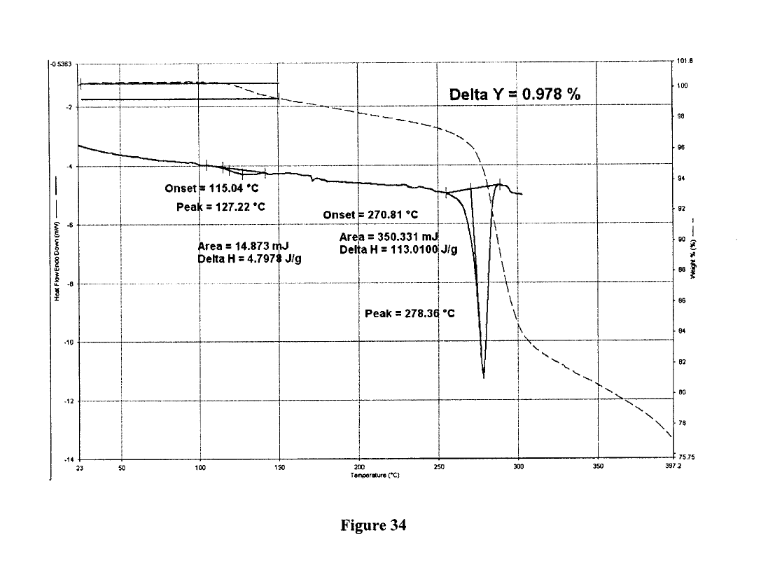

FIG. 33 shows the XRPD pattern for the Lestaurtinib Crystalline Form XIV. FIG. 34 shows the DSC/TGA overlay of the Lestaurtinib Crystalline Form XIV. Representative XRPD peaks for the Lestaurtinib Crystalline Form XIV solvate are listed in the following Table 16.

TABLE 16
Lestaurtinib Crystalline Form XIV XRPD peaks

| No. | Pos. [°2Th.] | d-spacing [Å] | Rel. Int. [%] |
|---|---|---|---|
| 1 | 6.82 | 12.94 | 54 |
| 2 | 7.75 | 11.39 | 78 |
| 3 | 8.51 | 10.37 | 40 |
| 4 | 9.68 | 9.12 | 11 |
| 5 | 9.85 | 8.96 | 15 |
| 6 | 11.97 | 7.38 | 26 |
| 7 | 12.20 | 7.24 | 33 |
| 8 | 13.19 | 6.70 | 64 |
| 9 | 14.21 | 6.22 | 83 |
| 10 | 14.67 | 6.03 | 99 |
| 11 | 15.06 | 5.87 | 62 |
| 12 | 15.48 | 5.71 | 68 |
| 13 | 16.90 | 5.23 | 37 |
| 14 | 17.17 | 5.15 | 9 |
| 15 | 17.55 | 5.04 | 66 |
| 16 | 17.92 | 4.94 | 52 |
| 17 | 18.52 | 4.78 | 7 |
| 18 | 19.14 | 4.63 | 9 |
| 19 | 19.35 | 4.58 | 8 |
| 20 | 19.60 | 4.52 | 9 |
| 21 | 19.94 | 4.44 | 8 |
| 22 | 20.46 | 4.33 | 11 |
| 23 | 21.52 | 4.12 | 9 |
| 24 | 21.98 | 4.04 | 7 |
| 25 | 22.88 | 3.88 | 12 |
| 26 | 23.02 | 3.86 | 10 |
| 27 | 25.13 | 3.53 | 100 |
| 28 | 25.80 | 3.45 | 32 |
| 29 | 26.33 | 3.38 | 35 |
| 30 | 26.58 | 3.35 | 40 |

In a preferred aspect of the present invention, representative XRPD peaks for the Lestaurtinib Crystalline Form XIV comprise one or more peaks selected from the group consisting of about 6.82, 7.75, 13.19, 14.21, 14.67, 15.06, 15.48, 17.55, 17.92 and 25.13 degrees 2-theta. In an even more preferred aspect, representative XRPD peaks for the Lestaurtinib Crystalline Form XIV comprise one or more peaks selected from the group consisting of about 7.75, 13.19, 14.21, 14.67, 17.55 and 25.13 degrees 2-theta.

### Example 16

### Lestaurtinib Crystalline Form XV (N,N. Dimethylacetamide Solvate)

A solution of lestaurtinib in N,N. dimethylacetamide was allowed to slowly evaporate to dryness under ambient conditions. The rate of evaporation was constrained by use of air tight film covers containing small holes.

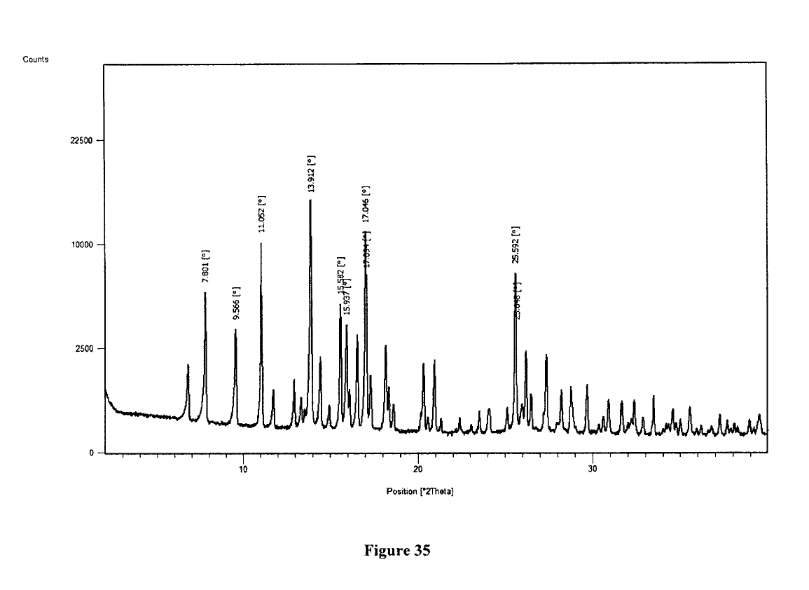

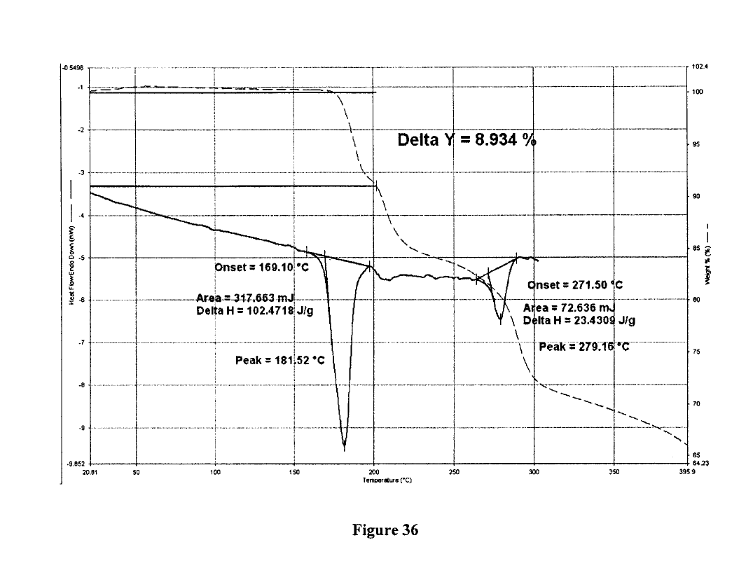

FIG. 35 shows the XRPD pattern for the Lestaurtinib Crystalline Form XV. FIG. 36 shows the DSC/TGA overlay of the Lestaurtinib Crystalline Form XV. Representative XRPD peaks for the Lestaurtinib Crystalline Form XV are listed in the following Table 17.

TABLE 17
Lestaurtinib Crystalline Form XV XRPD peaks

| No. | Pos. [°2Th.] | d-spacing [Å] | Rel. Int. [%] |
|---|---|---|---|
| 1 | 6.79 | 12.99 | 11 |
| 2 | 7.80 | 11.32 | 39. |
| 3 | 7.85 | 11.24 | 17 |
| 4 | 9.56 | 9.23 | 23 |
| 5 | 11.05 | 7.99 | 70 |
| 6 | 11.73 | 7.53 | 5 |
| 7 | 12.94 | 6.83 | 7. |
| 8 | 13.91 | 6.36 | 100 |
| 9 | 14.46 | 6.11 | 10 |
| 10 | 15.58 | 5.68 | 34 |
| 11 | 15.64 | 5.66 | 14 |
| 12 | 15.93 | 5.55 | 25 |
| 13 | 16.10 | 5.49 | 5 |
| 14 | 16.54 | 5.35 | 21 |
| 15 | 17.04 | 5.19 | 76 |
| 16 | 17.09 | 5.18 | 47 |
| 17 | 17.31 | 5.11 | 8 |
| 18 | 18.18 | 4.87 | 17 |
| 19 | 18.34 | 4.83 | 6 |
| 20 | 20.32 | 4.36 | 11 |
| 21 | 20.97 | 4.23 | 12 |
| 22 | 25.59 | 3.47 | 50 |
| 23 | 25.64 | 3.47 | 23 |
| 24 | 26.22 | 3.39 | 15 |
| 25 | 27.36 | 3.25 | 14 |
| 26 | 28.21 | 3.16 | 5 |
| 27 | 28.76 | 3.10 | 6 |
| 28 | 29.70 | 3.00 | 6 |

In a preferred aspect of the present invention, representative XRPD peaks for the Lestaurtinib Crystalline Form XV comprise one or more peaks selected from the group consisting of about 7.80, 9.56, 11.05, 13.91, 15.58, 15.93, 17.04, 17.09, 25.59 and 25.64 degrees 2-theta. In an even more preferred aspect, representative XRPD peaks for the Lestaurtinib Crystalline Form XV comprise one or more peaks selected from the group consisting of about 11.05, 13.91, 17.04, 17.09 and 25.59 2-theta.

### Example 17

### Lestaurtinib Crystalline Form XVI (2-pentanone/Water Solvate)

40 mg of amorphous form of lestaurtinib was slurried in 2-pentanone (20 volumes (100 mg in 2 mL)). The samples were heated at 49° C.-58° C. during 68 hours. The mixture was filtered through a 0.2μ nylon membrane filter. The solid was dried at 50° C. under house vacuum during 43 hours. The solid was isolated by filtration. The material was dried at 40° C. under house vacuum for 1 hour.

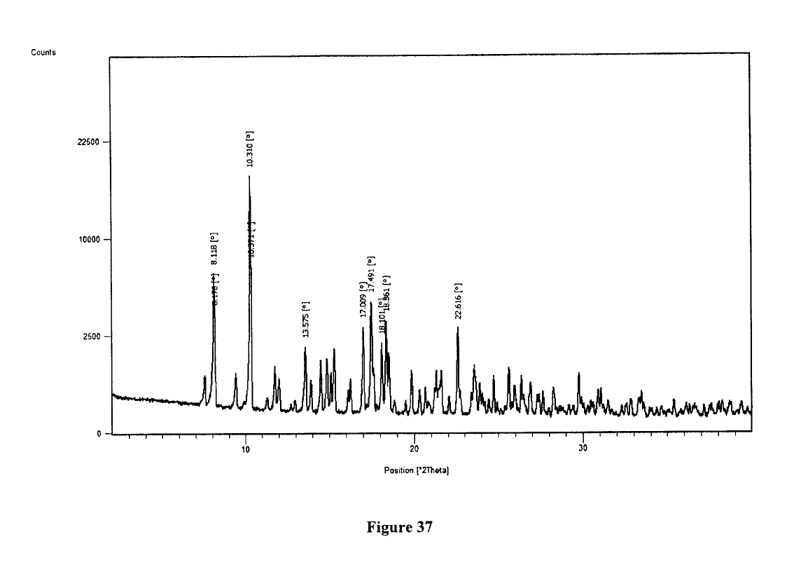

FIG. 37 shows the XRPD pattern for the Lestaurtinib Crystalline Form XVI. Representative XRPD peaks for the Lestaurtinib Crystalline Form XVI are listed in the following Table 18.

TABLE 18
Lestaurtinib Crystalline Form XVI XRPD peaks

| No. | Pos. [°2Th.] | d-spacing [Å] | Rel. Int. [%] |
|---|---|---|---|
| 1 | 8.12 | 10.88 | 38 |
| 2 | 8.18 | 10.80 | 20 |
| 3 | 10.31 | 8.57 | 100 |
| 4 | 10.37 | 8.52 | 41 |
| 5 | 11.76 | 7.52 | 6 |
| 6 | 13.58 | 6.52 | 10 |
| 7 | 13.63 | 6.49 | 6 |
| 8 | 14.49 | 6.11 | 7 |
| 9 | 14.86 | 5.96 | 8 |
| 10 | 15.30 | 5.79 | 10 |
| 11 | 17.01 | 5.21 | 16 |
| 12 | 17.49 | 5.07 | 26 |
| 13 | 17.62 | 5.03 | 6 |
| 14 | 18.10 | 4.90 | 11 |
| 15 | 18.36 | 4.83 | 18 |
| 16 | 18.53 | 4.78 | 9 |
| 17 | 19.86 | 4.47 | 5 |
| 18 | 21.33 | 4.16 | 5 |
| 19 | 21.62 | 4.11 | 5 |
| 20 | 22.62 | 3.93 | 16 |
| 21 | 23.58 | 3.77 | 6 |
| 22 | 25.61 | 3.48 | 6 |

In a preferred aspect of the present invention, representative XRPD peaks for the Lestaurtinib Crystalline Form XVI comprise one or more peaks selected from the group consisting of about 8.12, 8.18, 10.31, 10.37, 13.58, 17.01, 17.49, 18.10, 18.36 and 22.62 degrees 2-theta. In an even more preferred aspect, representative XRPD peaks for the Lestaurtinib Crystalline Form XVI comprise one or more peaks selected from the group consisting of about 8.12, 8.18, 10.31, 10.37 and 17.49 degrees 2-theta.

### Example 18

### Lestaurtinib Crystalline Form XVII (Crystalline Anhydrate 1)

40 mg of amorphous form of lestaurtinib was slurried in diisopropyl ether or methoxybenzene or methyl tert-butyl ether or 2-pentanone or 3-pentanone (10 volumes (40 mg in 400 μL)). The samples were heated from 20° C. to 80° C. at a rate of 4.8° C./min and after 30 minutes cooled at a slow rate (0.25° C./min) to a final temperature of 5° C. and kept at that temperature for 18 h. The solid was isolated by filtration. The material was dried at 40° C. under house vacuum during 1 hour.

In an alternative procedure, 40 mg of amorphous form of lestaurtinib was slurried in diisopropyl ether or 2-pentanone or 3-pentanone (10 volumes (40 mg in 400 μL)). The samples were heated from 20° C. to 80° C. at a rate of 4.8° C./min and after 30 minutes cooled at a fast rate (10° C./min) to a final temperature of 5° C. and kept at that temperature for 18 hours. The solid was isolated by filtration. The material was dried at 40° C. under house vacuum for 1 hour.

In an alternative procedure, 40 mg of amorphous form of lestaurtinib in 400 μL of solvent was slurried in diisopropyl ether or isopropyl acetate or methoxybenzene or 2-pentanone or 3-pentanone. These mixtures were slurried for 48 hours with alternating 4 hour periods at 50° C. and 5° C. (−0.5° C./min). The solid was isolated by filtration. The material was dried at 40° C. under house vacuum for 1 hour.

In an alternative procedure, 40 mg of amorphous form of lestaurtinib was added to a glass vial (2.0 mL, 32×11.6 mm). Chlorobenzene or toluene was added in 1.0 mL increments followed by heating with stirring to the boiling point until dissolved. The solution was not formed by the addition of a total of 10 mL of solvent, the mixture was syringe filtered (5μ Nylon membrane) and the solutions was allowed to slowly evaporate to dryness under ambient conditions. The solid was isolated by filtration.

In an alternative procedure, approximately 40 mg of amorphous form of lestaurtinib was added to a glass scintillation vial (20 mL, 26×56 mm). Chlorobenzene was added in 0.5 to 1.0 mL increments followed by heating with stirring to the boiling point until dissolved. If a solution was not formed by the addition of a total of 10 mL of solvent, the mixture was syringe filtered (5μ nylon membrane). One mL increments of the anti-solvent diisopropyl ether were then added to the solutions until the cloud point was reached. These mixtures were capped and allowed to cool to room temperature overnight and any solid that formed was isolated by suction filtration. The solid obtained was allowed to dry overnight in the fume hood. If no solid formed on adding 10 mL of antisolvent, the solution was allowed to evaporate in the fume hood until dry and any residue was examined by XRPD.

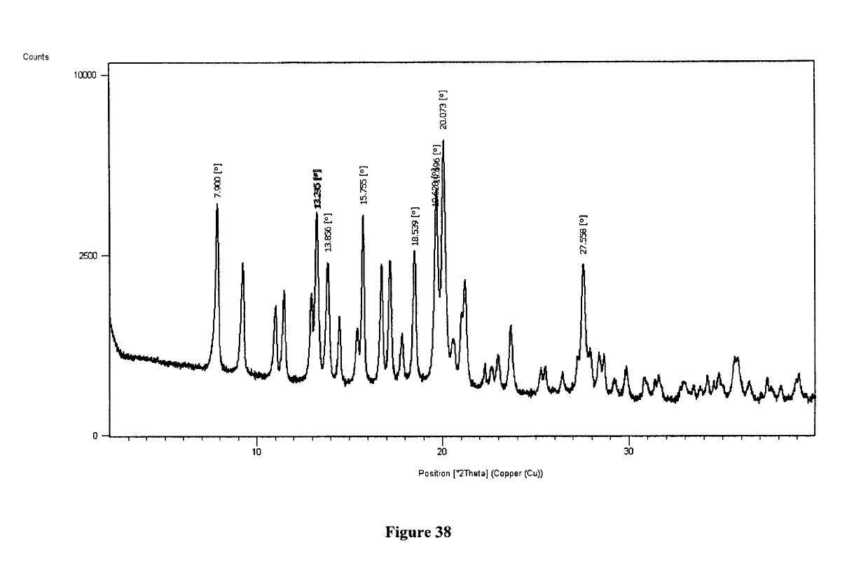

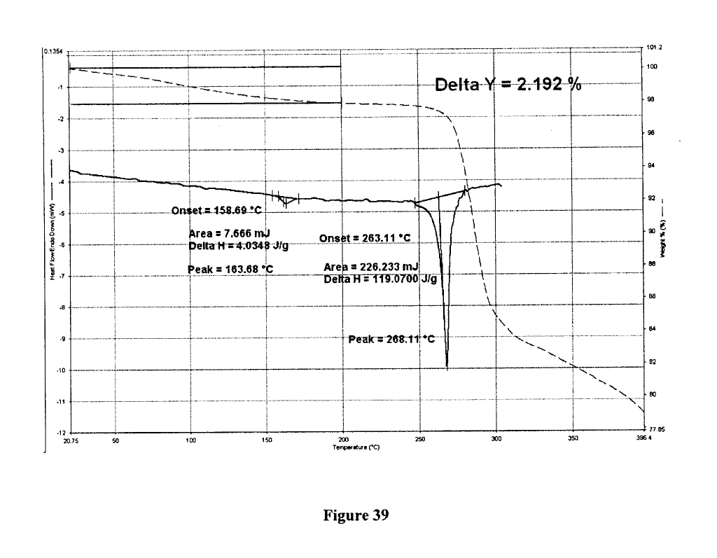

FIG. 38 shows the XRPD pattern for the Lestaurtinib Crystalline Form XVII. FIG. 39 shows the DSC/TGA overlay of the Lestaurtinib Crystalline Form XVII. Representative XRPD peaks for the Lestaurtinib Crystalline Form XVII are listed in the following Table 19.

TABLE 19
Lestaurtinib Crystalline Form XVII XRPD peaks

| No. | Pos. [°2Th.] | d-spacing [Å] | Rel. Int. [%] |
|---|---|---|---|
| 1 | 7.90 | 11.18 | 56 |
| 2 | 9.23 | 9.58 | 30 |
| 3 | 11.03 | 8.01 | 16 |
| 4 | 11.48 | 7.70 | 21 |
| 5 | 11.52 | 7.67 | 18 |
| 6 | 12.97 | 6.82 | 20 |
| 7 | 13.24 | 6.68 | 52 |
| 8 | 13.29 | 6.66 | 51 |
| 9 | 13.86 | 6.39 | 32 |
| 10 | 14.48 | 6.11 | 13 |
| 11 | 15.44 | 5.73 | 10 |
| 12 | 15.76 | 5.62 | 54 |
| 13 | 16.72 | 5.30 | 30 |
| 14 | 16.76 | 5.29 | 31 |
| 15 | 17.23 | 5.14 | 29 |
| 16 | 17.88 | 4.96 | 8 |
| 17 | 18.54 | 4.78 | 35 |
| 18 | 19.63 | 4.52 | 53 |
| 19 | 19.70 | 4.50 | 66 |
| 20 | 20.07 | 4.42 | 100 |
| 21 | 20.66 | 4.30 | 7 |
| 22 | 21.01 | 4.23 | 15 |
| 23 | 21.23 | 4.18 | 23 |
| 24 | 23.67 | 3.76 | 12 |
| 25 | 27.23 | 3.27 | 5 |
| 26 | 27.56 | 3.23 | 32 |
| 27 | 27.91 | 3.19 | 7 |
| 28 | 28.39 | 3.14 | 6 |
| 29 | 28.67 | 3.11 | 6 |
| 30 | 35.82 | 2.51 | 5 |

In a preferred aspect of the present invention, representative XRPD peaks for the Lestaurtinib Crystalline Form XVII comprise one or more peaks selected from the group consisting of about 7.90, 13.24, 13.29, 13.86, 15.76, 18.54, 19.63, 19.70, 20.07 and 27.56 degrees 2-theta. In an even more preferred aspect, representative XRPD peaks for the Lestaurtinib Crystalline Form XVII comprise one or more peaks selected from the group consisting of about 7.90, 15.76, 19.63, 19.70 and 20.07 degrees 2-theta.

### Example 19

### Lestaurtinib Crystalline Form XVIII (Crystalline Anhydrate 2)

Crystalline Form XVIII was obtained as 20 mg of Crystalline Form XVII was heated to 200° C. under nitrogen flow in an Anton Paar TK450 camera.

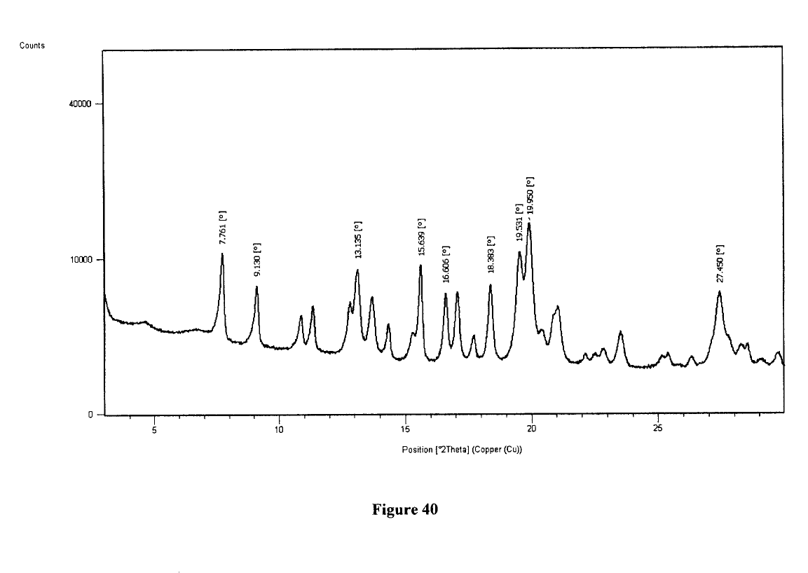

FIG. 40 shows the XRPD pattern for the Lestaurtinib Crystalline Form XVIII. Representative XRPD peaks for the Lestaurtinib Crystalline Form XVIII are listed in the following Table 20.

TABLE 20
Lestaurtinib Crystalline Form XVIII XRPD peaks

| No. | Pos. [°2Th.] | d-spacing [Å] | Rel. Int. [%] |
|---|---|---|---|
| 1 | 7.76 | 11.39 | 63 |
| 2 | 9.13 | 9.69 | 37 |
| 3 | 10.91 | 8.11 | 16 |
| 4 | 11.37 | 7.78 | 24 |
| 5 | 12.83 | 6.90 | 26 |
| 6 | 13.13 | 6.74 | 53 |
| 7 | 13.68 | 6.47 | 29 |
| 8 | 13.72 | 6.45 | 30 |
| 9 | 14.35 | 6.17 | 15 |
| 10 | 15.28 | 5.80 | 11 |
| 11 | 15.64 | 5.67 | 59 |
| 12 | 16.61 | 5.34 | 36 |
| 13 | 17.11 | 5.18 | 32 |
| 14 | 17.72 | 5.00 | 9 |
| 15 | 18.38 | 4.82 | 43 |
| 16 | 18.43 | 4.82 | 34 |
| 17 | 19.53 | 4.54 | 71 |
| 18 | 19.95 | 4.45 | 100 |
| 19 | 20.48 | 4.33 | 10 |
| 20 | 20.87 | 4.25 | 19 |
| 21 | 21.08 | 4.21 | 25 |
| 22 | 23.52 | 3.78 | 13 |
| 23 | 27.45 | 3.25 | 36 |
| 24 | 27.82 | 3.20 | 8 |
| 25 | 28.26 | 3.16 | 6 |
| 26 | 28.53 | 3.13 | 7 |

In a preferred aspect of the present invention, representative XRPD peaks for the Lestaurtinib Crystalline Form XVIII comprise one or more peaks selected from the group consisting of about 7.76, 9.13, 13.13, 15.64, 16.61, 18.38, 19.53, 19.95 and 27.45 degrees 2-theta. In an even more preferred aspect, representative XRPD peaks for the Lestaurtinib Crystalline Form XVIII comprise one or more peaks selected from the group consisting of about 7.76, 13.13, 15.64, 19.53 and 19.95 degrees 2-theta.

### Example 20

### Lestaurtinib Crystalline Form XIX (Crystalline Anhydrate 3)

Crystalline Form XIX was obtained as 20 mg of Crystalline Form XXIV was dried at room temperature during 3 days.

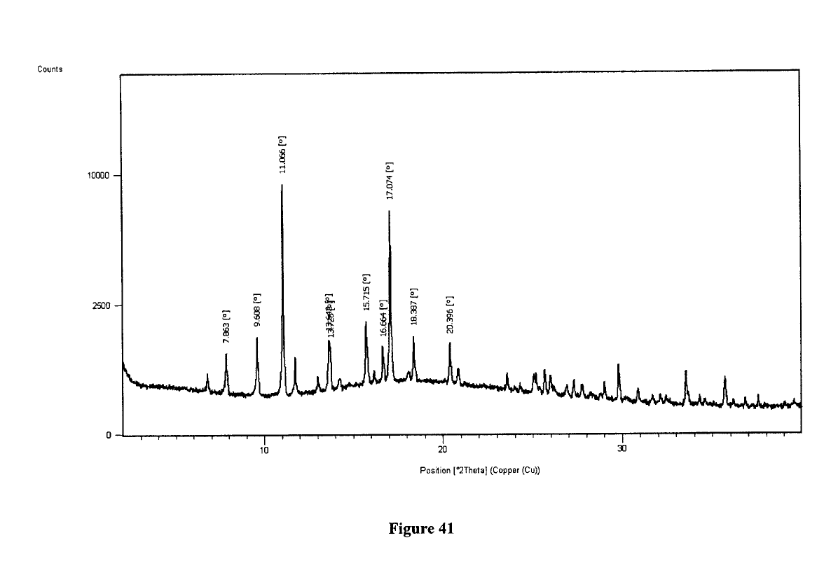

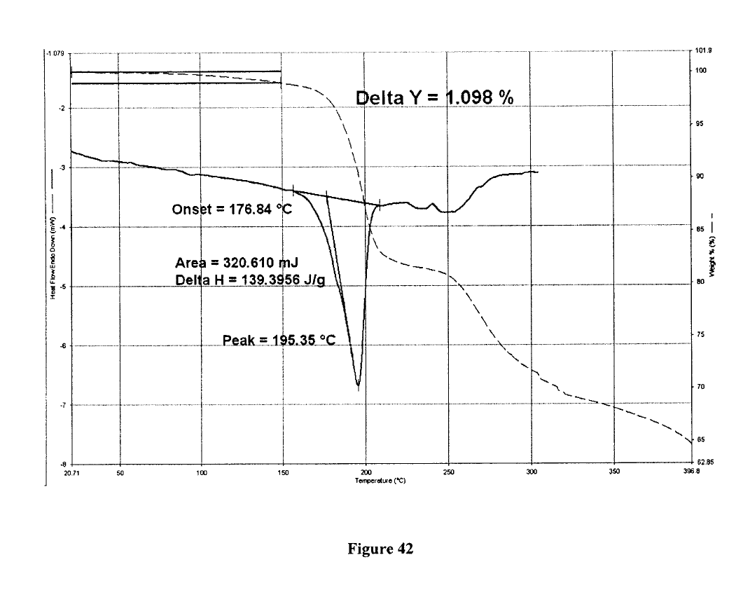

FIG. 41 shows the XRPD pattern for the Lestaurtinib Crystalline Form XIX. FIG. 42 shows the DSC/TGA overlay of the Lestaurtinib Crystalline Form XIX. Representative XRPD peaks for the Lestaurtinib Crystalline Form XIX are listed in the following Table 21.

TABLE 21
Lestaurtinib Crystalline Form XIX XRPD peaks

| No. | Pos. [°2Th.] | d-spacing [Å] | Rel. Int. [%] |
|---|---|---|---|
| 1 | 7.86 | 11.23 | 8 |
| 2 | 9.61 | 9.20 | 13 |
| 3 | 11.07 | 7.99 | 100 |
| 4 | 11.74 | 7.53 | 8 |
| 5 | 13.64 | 6.49 | 12 |
| 6 | 13.73 | 6.45 | 10 |
| 7 | 15.71 | 5.63 | 18 |
| 8 | 16.66 | 5.32 | 10 |
| 9 | 17.07 | 5.19 | 79 |
| 10 | 18.39 | 4.82 | 13 |
| 11 | 20.40 | 4.35 | 11 |
| 12 | 20.85 | 4.26 | 5 |
| 13 | 29.81 | 2.99 | 6 |

In a preferred aspect of the present invention, representative XRPD peaks for the Lestaurtinib Crystalline Form XIX comprise one or more peaks selected from the group consisting of about 7.86, 9.61, 11.07, 13.64, 13.73, 15.71, 16.66, 17.07, 18.39 and 20.40 degrees 2-theta. In an even more preferred aspect, representative XRPD peaks for the Lestaurtinib Crystalline Form XIX comprise one or more peaks selected from the group consisting of about 9.61, 11.07, 15.71, 17.07 and 18.39 degrees 2-theta.

### Example 21

### Lestaurtinib Crystalline Form XX (Butyronitrile/Water Solvate)

A solution of lestaurtinib in butyronitrile was allowed to slowly evaporate to dryness under ambient conditions. The rate of evaporation was constrained by use of air tight film covers containing small holes.

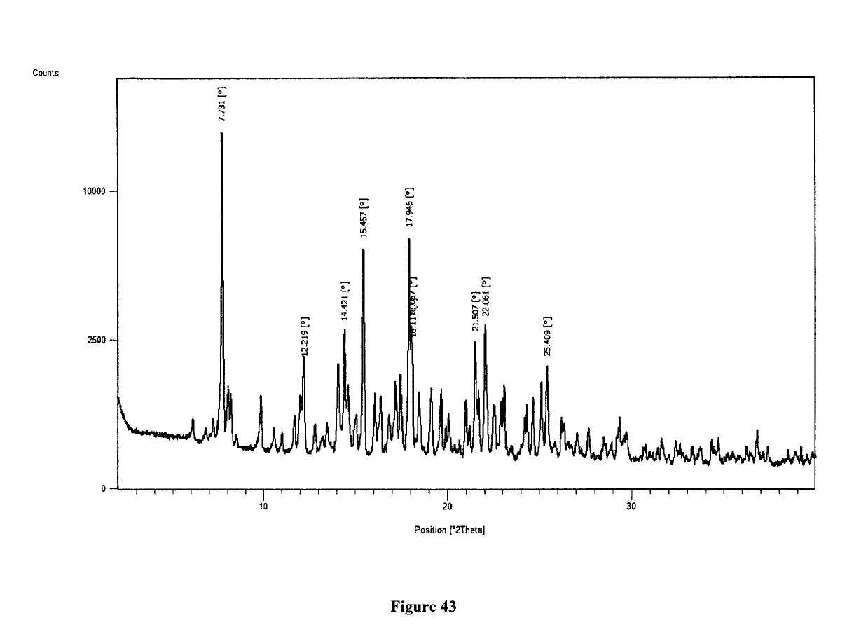

FIG. 43 shows the XRPD pattern for the Lestaurtinib Crystalline Form XX. Representative XRPD peaks for the Lestaurtinib Crystalline Form XX are listed in the following Table 22.

TABLE 22
Lestaurtinib Crystalline Form XX XRPD peaks

| No. | Pos. [°2Th.] | d-spacing [Å] | Rel. Int. [%] |
|---|---|---|---|
| 1 | 7.73 | 11.43 | 100 |
| 2 | 8.06 | 10.96 | 7 |
| 3 | 9.86 | 8.96 | 6 |
| 4 | 11.99 | 7.37 | 5 |
| 5 | 12.22 | 7.24 | 11 |
| 6 | 14.11 | 6.27 | 11 |
| 7 | 14.42 | 6.14 | 19 |
| 8 | 14.63 | 6.05 | 7 |
| 9 | 15.46 | 5.73 | 45 |
| 10 | 16.07 | 5.51 | 6 |
| 11 | 16.39 | 5.41 | 6 |
| 12 | 17.19 | 5.15 | 8 |
| 13 | 17.47 | 5.07 | 9 |
| 14 | 17.95 | 4.94 | 49 |
| 15 | 18.07 | 4.91 | 20 |
| 16 | 18.12 | 4.89 | 15 |
| 17 | 18.47 | 4.80 | 6 |
| 18 | 19.12 | 4.64 | 7 |
| 19 | 19.67 | 4.51 | 7 |
| 20 | 21.00 | 4.23 | 5 |
| 21 | 21.51 | 4.13 | 16 |
| 22 | 21.68 | 4.10 | 7 |
| 23 | 22.06 | 4.03 | 20 |
| 24 | 22.91 | 3.88 | 5 |
| 25 | 23.05 | 3.86 | 8 |
| 26 | 23.12 | 3.84 | 6 |
| 27 | 24.67 | 3.61 | 5 |
| 28 | 25.12 | 3.54 | 8 |
| 29 | 25.41 | 3.50 | 11 |

In a preferred aspect of the present invention, representative XRPD peaks for the Lestaurtinib Crystalline Form XX comprise one or more peaks selected from the group consisting of about 7.73, 12.22, 14.42, 15.46, 17.95, 18.07, 18.12, 21.51, 22.06 and 25.41 degrees 2-theta. In an even more preferred aspect, representative XRPD peaks for the Lestaurtinib Crystalline Form XX comprise one or more peaks selected from the group consisting of about 7.73, 15.46, 17.95, 18.07 and 22.06 degrees 2-theta.

### Example 22

### Lestaurtinib Crystalline Form XXI (N,N Dimethylformamide/Water Solvate)

A solution of lestaurtinib in N,N dimethylformamide was allowed to slowly evaporate to dryness under ambient conditions. The rate of evaporation was constrained by use of air tight film covers containing small holes.

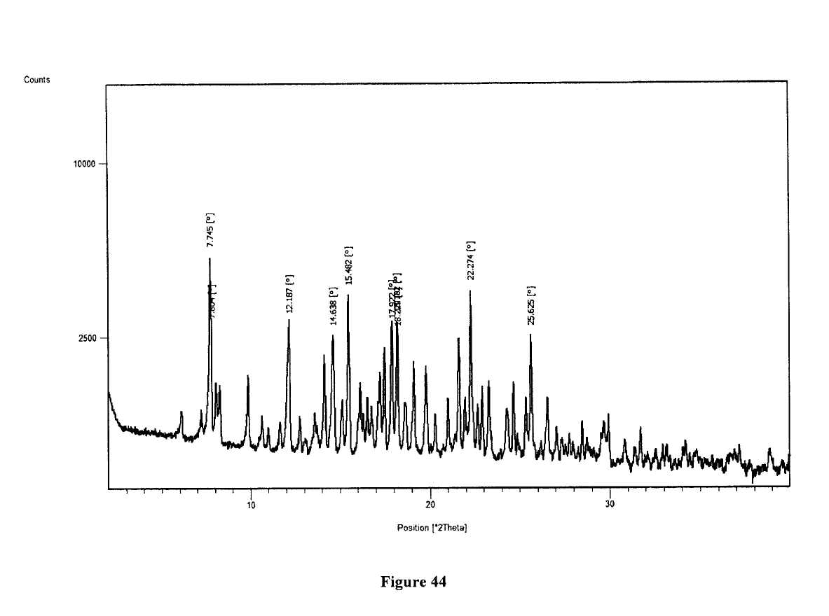

FIG. 44 shows the XRPD pattern for the Lestaurtinib Crystalline Form XXI. Representative XRPD peaks for the Lestaurtinib Crystalline Form XXI are listed in the following Table 23.

TABLE 23
Lestaurtinib Crystalline Form XXI XRPD peaks

| No. | Pos. [°2Th.] | d-spacing [Å] | Rel. Int. [%] |
|---|---|---|---|
| 1 | 7.74 | 11.41 | 100 |
| 2 | 7.80 | 11.32 | 45 |
| 3 | 8.07 | 10.95 | 17 |
| 4 | 8.28 | 10.67 | 17 |
| 5 | 9.86 | 8.97 | 21 |
| 6 | 12.06 | 7.33 | 23 |
| 7 | 12.19 | 7.26 | 49 |
| 8 | 14.14 | 6.26 | 32 |
| 9 | 14.59 | 6.07 | 37 |
| 10 | 14.64 | 6.05 | 42 |
| 11 | 15.48 | 5.72 | 70 |
| 12 | 15.55 | 5.70 | 32 |
| 13 | 16.13 | 5.49 | 19 |
| 14 | 17.22 | 5.14 | 22 |
| 15 | 17.45 | 5.08 | 27 |
| 16 | 17.49 | 5.07 | 35 |
| 17 | 17.92 | 4.95 | 46 |
| 18 | 18.18 | 4.88 | 51 |
| 19 | 18.23 | 4.86 | 42 |
| 20 | 19.11 | 4.64 | 29 |
| 21 | 19.79 | 4.48 | 26 |
| 22 | 21.61 | 4.11 | 41 |
| 23 | 21.66 | 4.10 | 29 |
| 24 | 22.27 | 3.99 | 73 |
| 25 | 22.92 | 3.88 | 18 |
| 26 | 23.28 | 3.82 | 20 |
| 27 | 24.64 | 3.61 | 19 |
| 28 | 25.33 | 3.51 | 14 |
| 29 | 25.62 | 3.47 | 43 |

In a preferred aspect of the present invention, representative XRPD peaks for the Lestaurtinib Crystalline Form XXI comprise one or more peaks selected from the group consisting of about 7.74, 7.80, 12.19, 14.64, 15.48, 17.92, 18.18, 18.23, 21.61 and 22.27 degrees 2-theta. In an even more preferred aspect, representative XRPD peaks for the Lestaurtinib Crystalline Form XXI comprise one or more peaks selected from the group consisting of about 7.74, 12.19, 15.48, 18.18, and 22.27 degrees 2-theta.

### Example 23

### Lestaurtinib Crystalline Form XXII (N-butyl Acetate Solvate)

40 mg of amorphous form of lestaurtinib in 400 μL of solvent was slurried in n-butyl acetate. These mixtures was slurried for 48 hours with alternating 4 hour periods at 50° C. and 5° C. (−0.5° C./min). The solid was isolated by filtration. The material was dried at 40° C. under house vacuum for 1 hour.

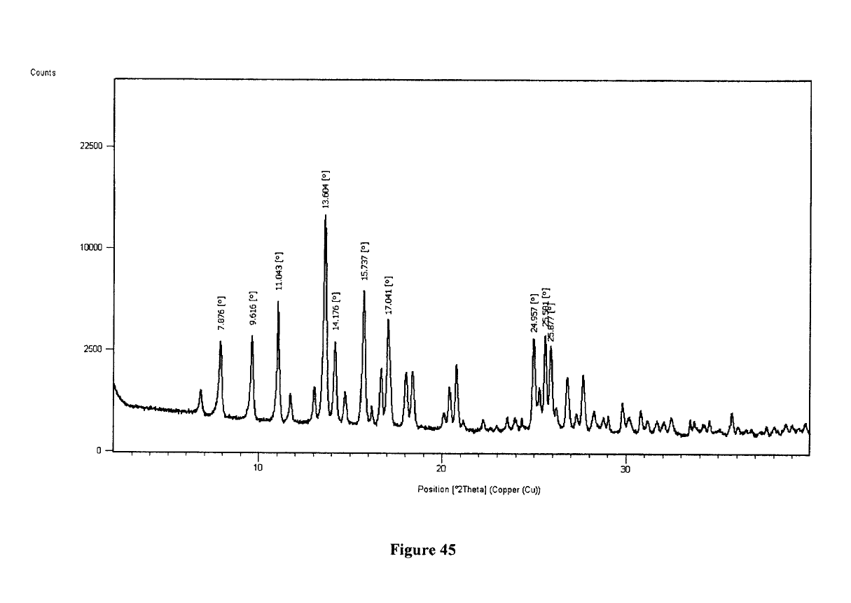

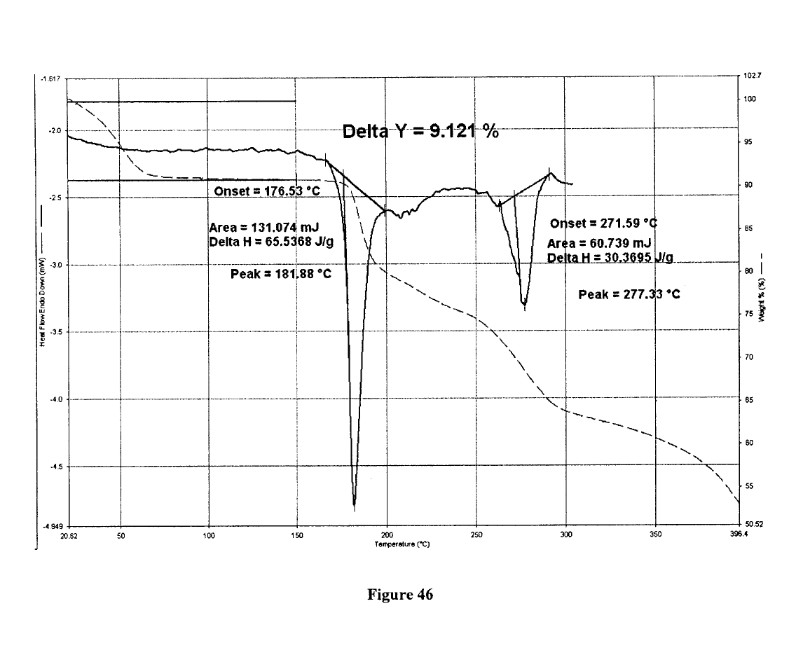

FIG. 45 shows the XRPD pattern for the Lestaurtinib Crystalline Form XXII. FIG. 46 shows the DSC/TGA overlay of the Lestaurtinib Crystalline Form XXII. Representative XRPD peaks for the Lestaurtinib Crystalline Form XXII are listed in the following Table 24.

TABLE 24
Lestaurtinib Crystalline Form XXII XRPD peaks

| No. | Pos. [°2Th.] | d-spacing [Å] | Rel. Int. [%] |
|---|---|---|---|
| 1 | 7.88 | 11.22 | 20 |
| 2 | 9.62 | 9.19 | 23 |
| 3 | 11.04 | 8.01 | 40 |
| 4 | 13.02 | 6.79 | 6 |
| 5 | 13.60 | 6.50 | 100 |
| 6 | 14.18 | 6.24 | 21 |
| 7 | 14.72 | 6.01 | 5 |
| 8 | 15.74 | 5.63 | 47 |
| 9 | 16.70 | 5.30 | 11 |
| 10 | 17.04 | 5.20 | 29 |
| 11 | 18.06 | 4.91 | 11 |
| 12 | 18.36 | 4.83 | 10 |
| 13 | 18.45 | 4.80 | 9 |
| 14 | 20.38 | 4.35 | 6 |
| 15 | 20.76 | 4.27 | 13 |
| 16 | 24.96 | 3.56 | 22 |
| 17 | 25.29 | 3.52 | 7 |
| 18 | 25.58 | 3.48 | 24 |
| 19 | 25.88 | 3.44 | 18 |
| 20 | 26.83 | 3.32 | 10 |
| 21 | 27.68 | 3.22 | 10 |

In a preferred aspect of the present invention, representative XRPD peaks for the Lestaurtinib Crystalline Form XXII comprise one or more peaks selected from the group consisting of about 7.88, 9.62, 11.04, 13.60, 14.18, 15.74, 17.04, 24.96, 25.58 and 25.88 degrees 2-theta. In an even more preferred aspect, representative XRPD peaks for the Lestaurtinib Crystalline Form XXII comprise one or more peaks selected from the group consisting of about 11.04, 13.60, 15.74, 17.04 and 25.58 degrees 2-theta.

### Example 24

### Lestaurtinib Crystalline Form XXIII (2:3 Formamide Solvate)

A solution of lestaurtinib in formamide was allowed to slowly evaporate to dryness under ambient conditions. The rate of evaporation was constrained by use of air tight film covers containing small holes.

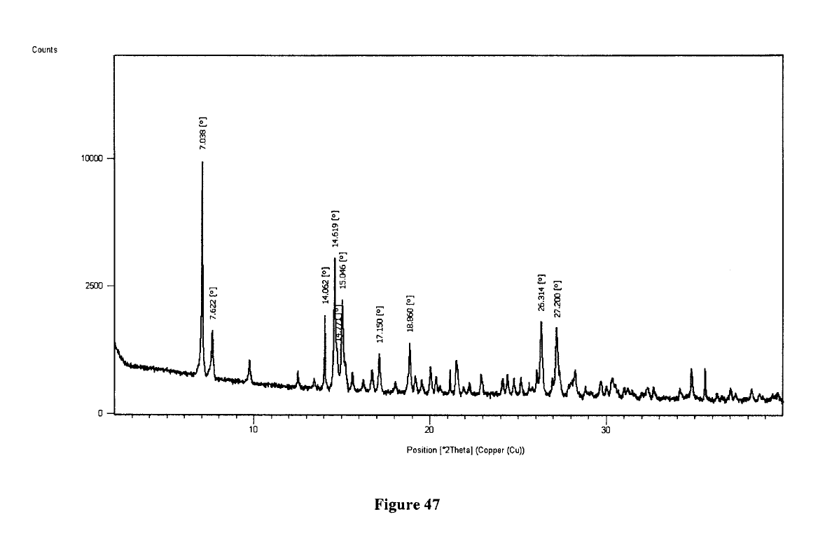

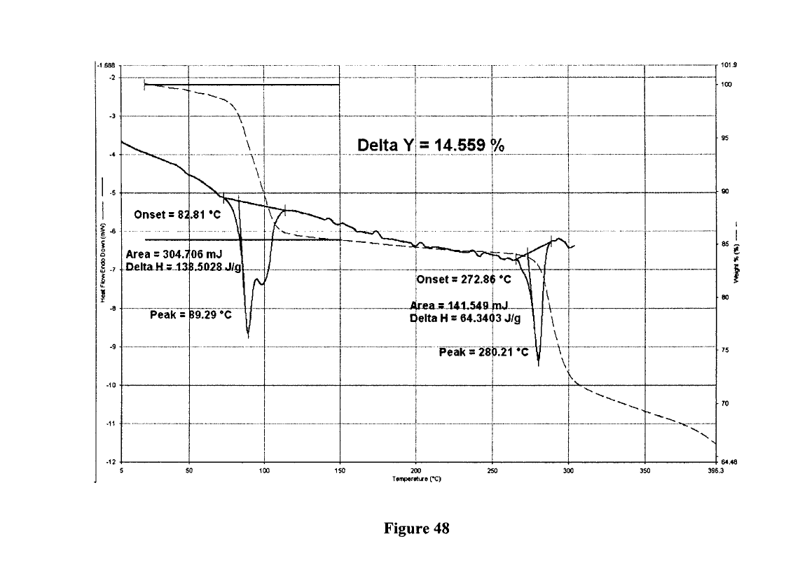

FIG. 47 shows the XRPD pattern for the Lestaurtinib Crystalline Form XXIII. FIG. 48 shows the DSC/TGA overlay of the Lestaurtinib Crystalline Form XXIII. Representative XRPD peaks for the Lestaurtinib Crystalline Form XXIII are listed in the following Table 25.

TABLE 25
Lestaurtinib Crystalline Form XXIII XRPD peaks

| No. | Pos. [°2Th.] | d-spacing [Å] | Rel. Int. [%] |
|---|---|---|---|
| 1 | 7.03 | 12.54 | 100 |
| 2 | 7.62 | 11.58891 | 9 |
| 3 | 14.06 | 6.29 | 14 |
| 4 | 14.61 | 6.05 | 37 |
| 5 | 14.77 | 5.99 | 5 |
| 6 | 15.04 | 5.88 | 19 |
| 7 | 17.14 | 5.16 | 5 |
| 8 | 18.85 | 4.70 | 7 |
| 9 | 26.31 | 3.38 | 13 |
| 10 | 27.20 | 3.278 | 11 |

In a preferred aspect of the present invention, representative XRPD peaks for the Lestaurtinib Crystalline Form XXIII comprise one or more peaks selected from the group consisting of about 7.03, 7.62, 14.06, 14.61, 14.77, 15.04, 17.14, 18.85, 26.31 and 27.20 degrees 2-theta. In an even more preferred aspect, representative XRPD peaks for the Lestaurtinib Crystalline Form XXIII comprise one or more peaks selected from the group consisting of about 7.03, 14.06, 14.61, 15.04 and 26.31 degrees 2-theta.

### Example 25

### Lestaurtinib Crystalline Form XXIV (1:3 Methanol Solvate)

Approximately 40 mg of Form I of lestaurtinib was slurried in methanol (10 volumes (40 mg in 400 μL)). The sample was heated from 20° C. to 80° C. at a rate of 4.8° C./min and after 30 minutes cooled at a slow rate (0.25° C./min) to a final temperature of 5° C. and kept at that temperature for 18 h. The solid was isolated by filtration. The material was dried at 40° C. under house vacuum for 1 hour.

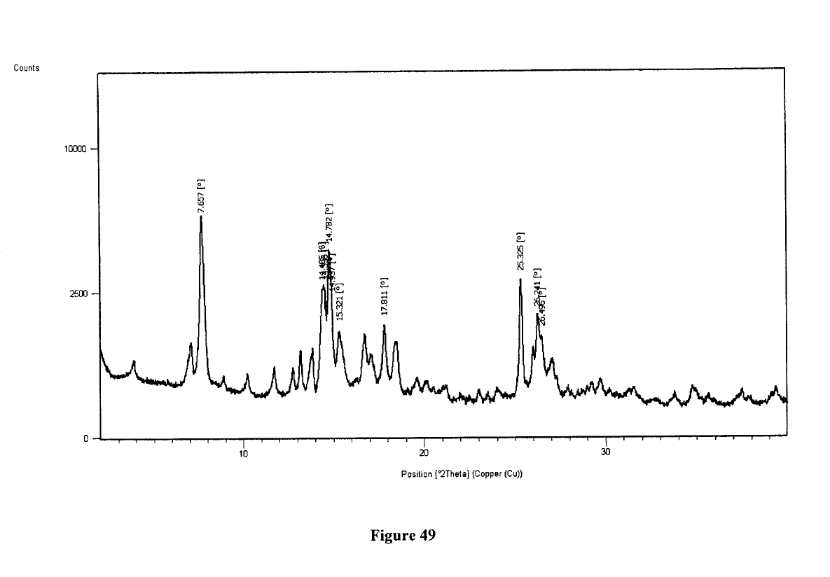

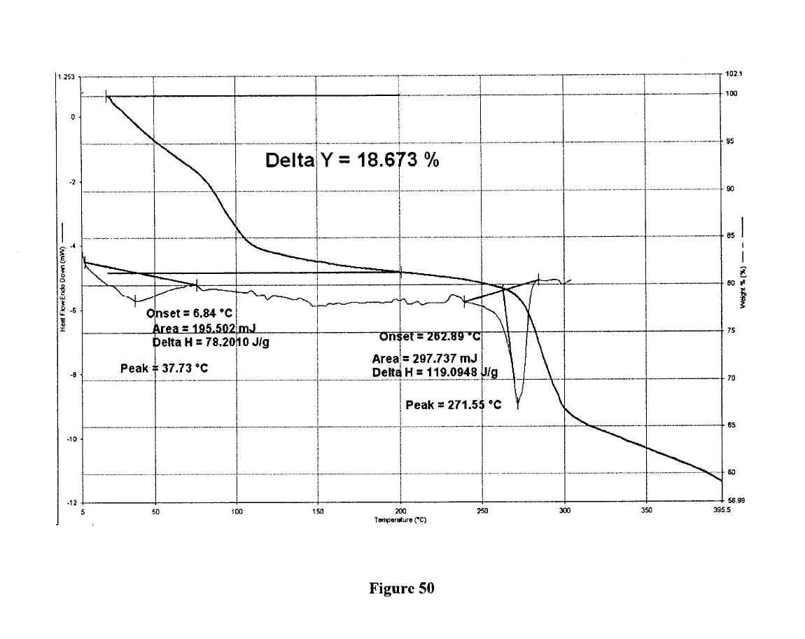

FIG. 49 shows the XRPD pattern for the Lestaurtinib Crystalline form XXIV. FIG. 50 shows the DSC/TGA overlay of the Lestaurtinib Crystalline form XXIV. Representative XRPD peaks for the Lestaurtinib Crystalline form XXIV are listed in the following Table 26.

TABLE 26
Lestaurtinib Crystalline Form XXIV XRPD peaks

| No. | Pos. [°2Th.] | d-spacing [Å] | Rel. Int. [%] |
|---|---|---|---|
| 1 | 3.90 | 22.63 | 5 |
| 2 | 7.10 | 12.44 | 13 |
| 3 | 7.66 | 11.54 | 100 |
| 4 | 10.24 | 8.63 | 5 |
| 5 | 11.74 | 7.53 | 7 |
| 6 | 12.78 | 6.92 | 6 |
| 7 | 13.21 | 6.70 | 12 |
| 8 | 13.86 | 6.39 | 14 |
| 9 | 14.40 | 6.14 | 44 |
| 10 | 14.54 | 6.09 | 45 |
| 11 | 14.78 | 5.99 | 73 |
| 12 | 14.94 | 5.93 | 36 |
| 13 | 15.32 | 5.78 | 19 |
| 14 | 16.75 | 5.29 | 17 |
| 15 | 17.08 | 5.19 | 9 |
| 16 | 17.81 | 4.98 | 22 |
| 17 | 18.35 | 4.83 | 12 |
| 18 | 18.52 | 4.79 | 14 |
| 19 | 25.32 | 3.51 | 51 |
| 20 | 25.98 | 3.43 | 13 |
| 21 | 26.24 | 3.39 | 28 |
| 22 | 26.50 | 3.36 | 18 |
| 23 | 27.08 | 3.29 | 10 |

In a preferred aspect of the present invention, representative XRPD peaks for the Lestaurtinib Crystalline Form XXIV comprise one or more peaks selected from the group consisting of about 7.66, 14.40, 14.54, 14.78, 14.94, 15.32, 17.81, 25.32, 26.24 and 26.50 degrees 2-theta. In an even more preferred aspect, representative XRPD peaks for the Lestaurtinib Crystalline Form XXIV comprise one or more peaks selected from the group consisting of about 7.66, 14.40, 14.54, 14.78 and 25.32 degrees 2-theta.

### Example 26

### Lestaurtinib Crystalline Form XXV (N-methylpyrrolidinone Solvate)

A solution of lestaurtinib in N-methylpyrrolidinone was allowed to slowly evaporate to dryness under ambient conditions. The rate of evaporation was constrained by use of air tight film covers containing small holes.

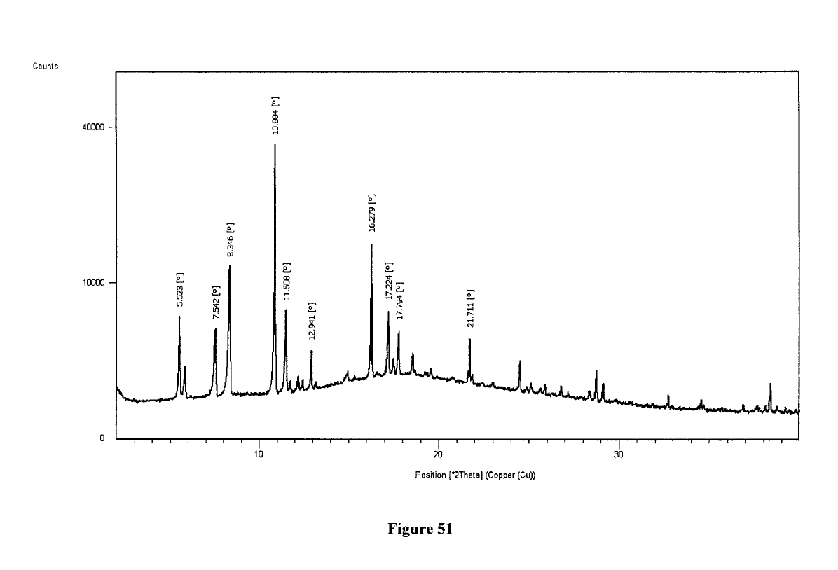

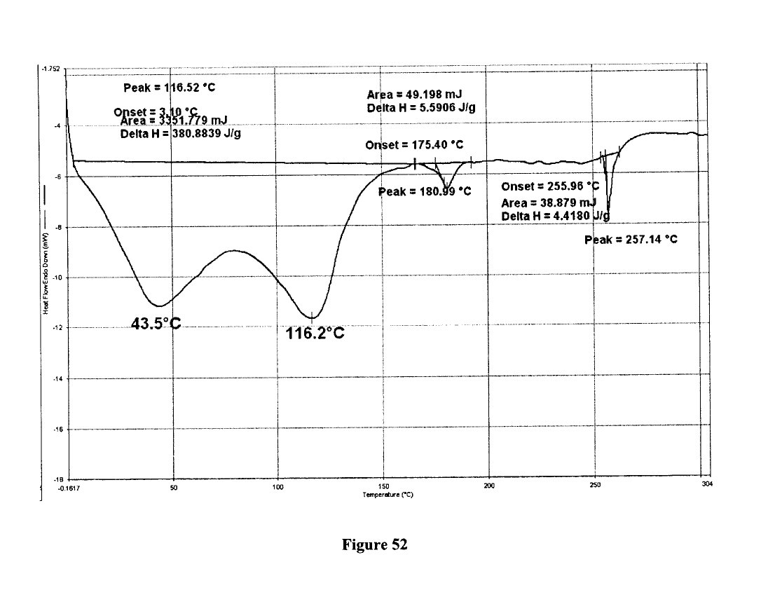

FIG. 51 shows the XRPD pattern for the Lestaurtinib Crystalline Form XXV. FIG. 52 shows the DSC/TGA overlay of the Lestaurtinib Crystalline Form XXV. Representative XRPD peaks for the Lestaurtinib Crystalline Form XXV are listed in the following Table 27.

TABLE 27
6/27 Lestaurtinib Crystalline Form XXV XRPD peaks

| No. | Pos. [°2Th.] | d-spacing [Å] | Rel. Int. [%] |
|---|---|---|---|
| 1 | 5.52 | 15.99 | 16 |
| 2 | 7.54 | 11.71 | 12 |
| 3 | 8.35 | 10.59 | 33 |
| 4 | 10.88 | 8.12 | 100 |
| 5 | 11.51 | 7.68 | 18 |
| 6 | 12.94 | 6.84 | 8 |
| 7 | 16.28 | 5.44 | 44 |
| 8 | 17.22 | 5.14 | 18 |
| 9 | 17.51 | 5.06 | 6 |
| 10 | 17.79 | 4.98 | 12 |
| 11 | 18.57 | 4.77 | 7 |
| 12 | 21.71 | 4.09 | 11 |
| 13 | 24.53 | 3.63 | 6 |

In a preferred aspect of the present invention, representative XRPD peaks for the Lestaurtinib Crystalline Form XXV comprise one or more peaks selected from the group consisting of about 5.52, 7.54, 8.35, 10.88, 11.51, 12.94, 16.28, 17.22, 17.79, and 21.71 degrees 2-theta. In an even more preferred aspect, representative XRPD peaks for the Lestaurtinib Crystalline Form XXV comprise one or more peaks selected from the group consisting of about 5.52, 8.35, 10.88, 11.51 and 16.28 degrees 2-theta.

### Example 27

### Lestaurtinib Crystalline Form XXVI (1-2 Dichloroethane Solvate)

40 mg of amorphous form of lestaurtinib was added to a glass vial (2.0 mL, 32×11.6 mm). 1-2 dichloroethane was added in 1.0 mL increments followed by heating with stirring to the boiling point until dissolved. The solution was not formed by the addition of a total of 10 mL of solvent, the mixture was syringe filtered (5μ Nylon membrane) and the solution was allowed to slowly evaporate to dryness under ambient conditions.

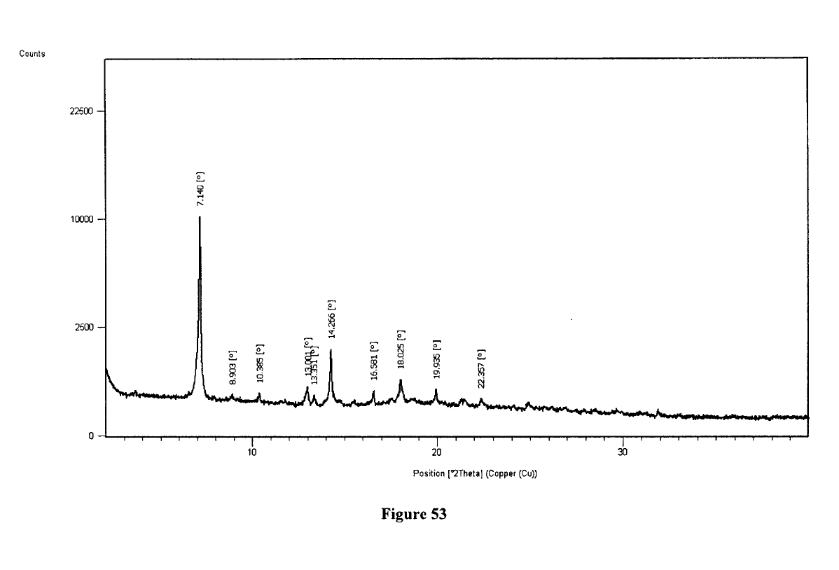

FIG. 53 shows the XRPD pattern for the Lestaurtinib Crystalline Form XXVI. Representative XRPD peaks for the Lestaurtinib Crystalline Form XXVI are listed in the following Table 28.

TABLE 28
Lestaurtinib Crystalline Form XXVI XRPD peaks

| No. | Pos. [°2Th.] | d-spacing [Å] | Rel. Int. [%] |
|---|---|---|---|
| 1 | 3.58 | 24.63 | 1 |
| 2 | 7.14 | 12.37 | 100 |
| 3 | 8.90 | 9.92 | 1 |
| 4 | 10.39 | 8.51 | 1 |
| 5 | 13.00 | 6.80 | 3 |
| 6 | 13.35 | 6.63 | 1 |
| 7 | 14.27 | 6.20 | 14 |
| 8 | 16.58 | 5.34 | 2 |
| 9 | 17.55 | 5.05 | 1 |
| 10 | 18.02 | 4.92 | 5 |
| 11 | 18.71 | 4.74 | 1 |
| 12 | 19.94 | 4.45 | 3 |
| 13 | 21.33 | 4.16 | 1 |

In a preferred aspect of the present invention, representative XRPD peaks for the Lestaurtinib Crystalline Form XXVI comprise one or more peaks selected from the group consisting of about 7.14, 8.90, 10.39, 13.00, 13.35, 14.27, 16.58, 18.02, 19.94 and 21.33 degrees 2-theta. In an even more preferred aspect, representative XRPD peaks for the Lestaurtinib Crystalline Form XXVI comprise one or more peaks selected from the group consisting of about 7.14, 13.00, 14.27, 18.02 and 19.94 degrees 2-theta.

### Example 28

### Lestaurtinib Crystalline Form XXVII (Proplyene Carbonate Solvate)

40 mg of amorphous form of lestaurtinib was added to a glass vial. Propylene carbonate was added in 1.0 mL increments followed by heating with stirring to the boiling point until dissolved. The solution was not formed by the addition of a total of 10 mL of solvent, the mixture was syringe filtered (5μ Nylon membrane) and the solutions was allowed to slowly evaporate to dryness under ambient conditions.

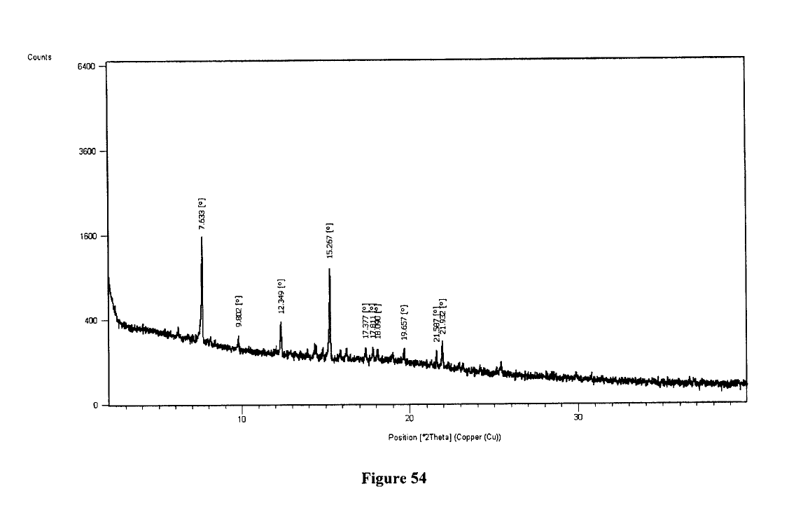

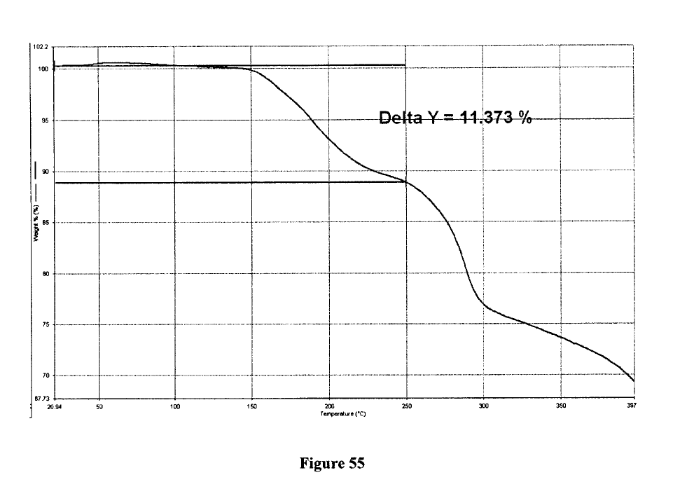

FIG. 54 shows the XRPD pattern for the Lestaurtinib Crystalline Form XXVII. FIG. 55 shows the DSC/TGA overlay of the Lestaurtinib Crystalline Form XXVII. Representative XRPD peaks for the Lestaurtinib Crystalline Form XXVII are listed in the following Table 29.

TABLE 29
Lestaurtinib Crystalline Form XXVII XRPD peaks

| No. | Pos. [°2Th.] | d-spacing [Å] | Rel. Int. [%] |
|---|---|---|---|
| 1 | 7.63 | 11.57 | 100 |
| 2 | 9.80 | 9.02 | 7 |
| 3 | 12.35 | 7.16 | 17 |
| 4 | 15.27 | 5.80 | 69 |
| 5 | 19.66 | 4.51 | 5 |
| 6 | 21.59 | 4.11 | 5 |
| 7 | 21.93 | 4.05 | 9 |

In a preferred aspect of the present invention, representative XRPD peaks for the Lestaurtinib Crystalline Form XXVII comprise one or more peaks selected from the group consisting of about 7.63, 9.80, 12.35, 15.27, 19.66, 21.59 and 21.93 degrees 2-theta. In an even more preferred aspect, representative XRPD peaks for the Lestaurtinib Crystalline Form XXVII comprise one or more peaks selected from the group consisting of about 7.63, 9.80, 12.35, 15.27 and 21.93 degrees 2-theta.

### Example 29

### Lestaurtinib Crystalline Form XXVIII (1:2 Acetic Acid Solvate)

40 mg of a ground lestaurtinib sample was weighed into a small, screw top vial. 1 ml of acetic acid was added and the vial was subjected to heat/cool cycles between ambient and 50° C. (4 hours hot, 4 hours cool) for 24 hours. Solid was isolated by filtration and air dried for 1 hour before analysis.

In an alternative procedure, 40 mg of amorphous form of lestaurtinib in 1 mL of solvent was slurried in acetic acid. These mixtures was slurried for 24 hours with alternating 4 hour periods at 50° C. and 5° C. (−0.5° C./min). The solid was isolated by filtration. The material was dried at 40° C. under house vacuum for 1 hour.

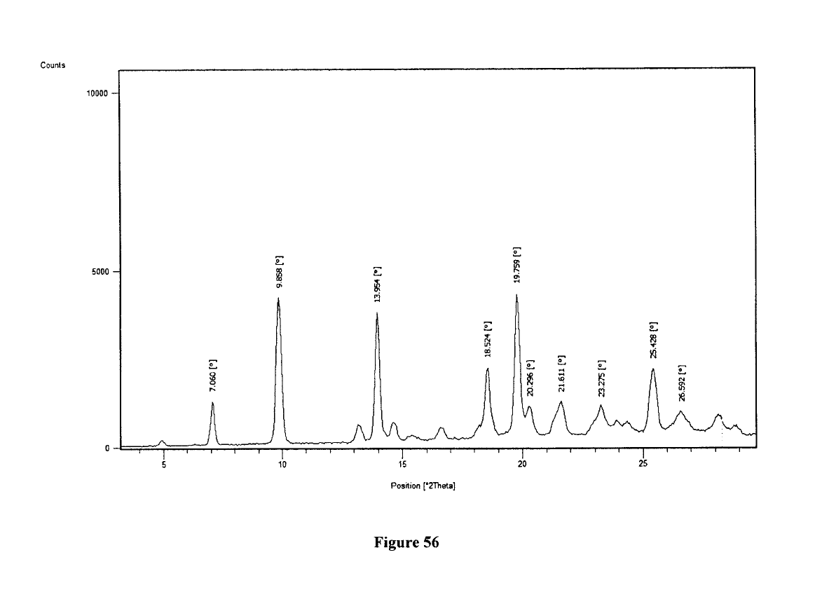

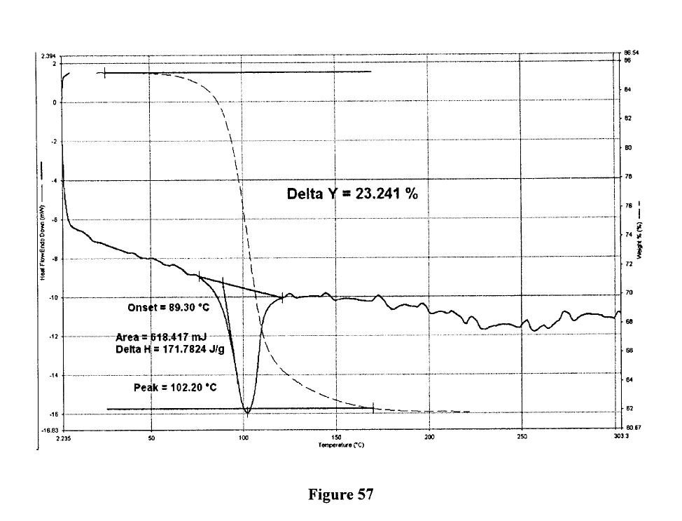

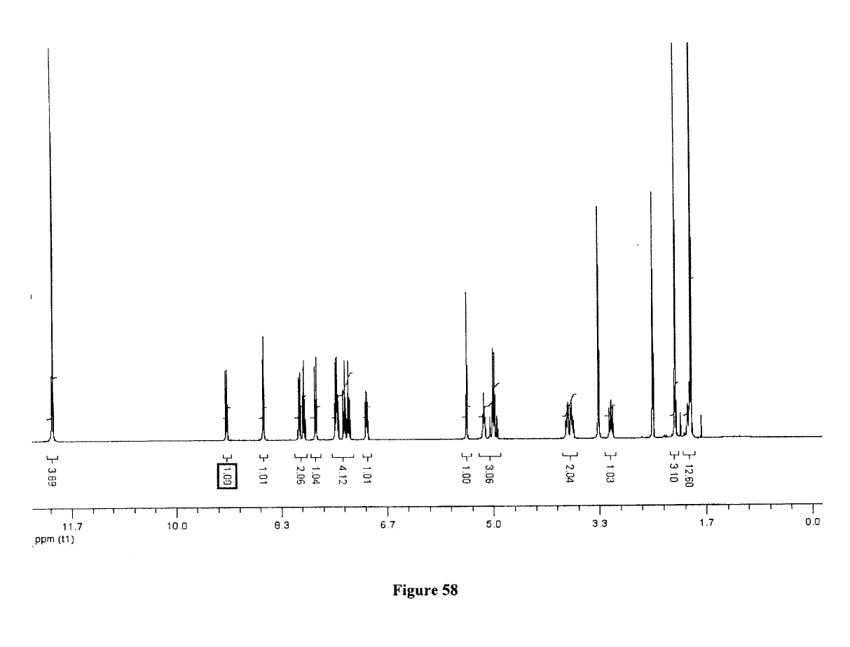

FIG. 56 shows the XRPD pattern for the Lestaurtinib Crystalline Form XXVIII. FIG. 57 shows the DSC/TGA overlay of the Lestaurtinib Crystalline Form XXVIII. FIG. 58 shows the ¹H NMR spectrum for the Lestaurtinib Crystalline Form XXVIII. Representative XRPD peaks for the Lestaurtinib Crystalline Form XXVIII are listed in the following Table 30.

TABLE 30
Lestaurtinib Crystalline Form XXVIII XRPD peaks

| No. | Pos. [°2Th.] | d-spacing [Å] | Rel. Int. [%] |
|---|---|---|---|
| 1 | 7.06 | 12.52 | 30 |
| 2 | 9.86 | 8.97 | 100 |
| 3 | 13.17 | 6.72 | 12 |
| 4 | 13.95 | 6.35 | 88 |
| 5 | 14.63 | 6.06 | 12 |
| 6 | 16.60 | 5.34 | 8 |
| 7 | 18.52 | 4.79 | 46 |
| 8 | 19.76 | 4.49 | 96 |
| 9 | 20.30 | 4.38 | 19 |
| 10 | 21.61 | 4.11 | 22 |
| 11 | 23.28 | 3.82 | 18 |
| 12 | 23.93 | 3.72 | 7 |
| 13 | 24.39 | 3.65 | 5 |
| 14 | 25.43 | 3.50 | 42 |
| 15 | 26.59 | 3.35 | 12 |
| 16 | 28.18 | 3.17 | 11 |
| 17 | 28.87 | 3.09 | 5 |

In a preferred aspect of the present invention, representative XRPD peaks for the Lestaurtinib Crystalline Form XXVIII comprise one or more peaks selected from the group consisting of about 7.06, 9.86, 13.95, 18.52, 19.76, 20.30, 21.61, 23.28, 25.43 and 26.59 degrees 2-theta. In an even more preferred aspect, representative XRPD peaks for the Lestaurtinib Crystalline Form XXVIII comprise one or more peaks selected from the group consisting of about 9.86, 13.95, 18.52, 19.76 and 25.43 degrees 2-theta.

### Crystalline Structure

### Example 30

### Lestaurtinib Single Crystal Form VI (1:1 Methanol Solvate)

These single crystal diffraction studies were conducted on a crystalline specimen of lestaurtinib prepared by dissolving 22.4 mg of lestaurtinib in 5 ml of methanol. The sample was heated with stirring to 60° C. for 5 minutes, and then at the boiling point for a total heating and stirring time of 60 minutes. All of the solid did not dissolve. The solution was then filtered through cotton and left to evaporate. After one month, crystals had formed in the vial. There is one independent molecule of lestaurtinib in the asymmetric unit. They are linked by hydrogen bonds involving the methanol and by hydrogen bonds between the molecules themselves.

| Crystal system | Monoclinic | |
|---|---|---|
| Space group | P2₁ | |
| Unit cell dimensions, Cu | a = 11.5559(4) Å | α = 90° |
| | b = 6.7675(2) Å | β = 112.397(1)° |
| | c = 14.8765(6) Å | γ = 90° |
| Volume | 1082.08(12) Å³ | |
| Z | 2 | |

### Example 31

Lestaurtinib Single Crystal Form VII (propionitrile/water solvate 0.4419 grams of lestaurtinib in 50 mL of propionitrile was stirred with heating to the boiling point and syringe filtered to give a clear solution that was evaporated with heating to 5-10 mL. The initially clear, yellow solution was allowed to stand in the refrigerator for about 120 hours. The supernatant liquid was decanted and the solid was allowed to dry to constant weight in the fume hood to yield 149.4 mg (32%) of white, crystalline solid. There are four independent molecules of CEP701, two molecules of propionitrile and one molecule of water in the asymmetric unit.

| Crystal system | Monoclinic | |
|---|---|---|
| Space group | P2₁ | |
| Unit cell dimensions, Mo | a = 13.448(3) Å | α = 90° |
| | b = 22.896(5) Å | β = 113.21(3)° |
| | c = 15.737(3) Å | γ = 90° |
| Volume | 4453.4(15) Å³ | |
| Z | 2 | |

### Example 32

### Lestaurtinib Single Crystal Form VIII (Acetone/Water Solvate)

1.0 grams of lestaurtinib in 90 mL of acetone was stirred with heating to the boiling point and held at the boiling point for 2-3 minutes until no more solid dissolved.

This warm mixture was syringe filtered and resulting yellow solution concentrated by evaporation to about 40 mL. Crystals began to form on the laboratory bench almost as soon as the solution was removed from the hot plate. The solution was chilled in the refrigerator over approximately 65 hours. The supernatant liquid was decanted and crystals removed to weighing paper and allowed to dry to constant weight in the fume hood to constant weight to give 444 mg (44% yield). There are four independent molecules of lestaurtinib, two of acetone and one of water in the asymmetric unit.

| Crystal system | Monoclinic | |
|---|---|---|
| Space group | P2₁ | |
| Unit cell dimensions, Mo | a = 13.6466(7) Å | α = 90° |
| | b = 22.7237(7) Å | β = 112.738(4) (6)° |
| | c = 15.8305(7) Å | γ = 90° |
| Volume | 4527.5(3) Å³ | |
| Z | 2 | |

### Example 33

### Lestaurtinib Single Crystal Form XV (N,N. Dimethylacetamide Solvate)

A solution of lestaurtinib in N,N. dimethylacetamide was allowed to slowly evaporate to dryness under ambient conditions. The rate of evaporation was constrained by use of air tight film covers containing small holes. There is one independent molecule of lestaurtinib in the asymmetric unit. They are linked into dimers by head to head hydrogen bonds between the amide moieties in the central core of the molecule. In addition the dimers are linked by hydrogen bonding between the amino-pyrimidine moieties.

| Crystal system | Orthorhombic | |
|---|---|---|
| Space group | P 2₁ 2₁ 2₁ | |
| Unit cell dimensions, Mo | a = 6.842 Å | α = 90° |
| | b = 15.918 Å | β = 90° |
| | c = 22.796 Å | γ = 90° |
| Volume | 2482.7 Å³ | |
| Z | 4 | |

### Example 34

### Lestaurtinib Single Crystal Form XXI (N,N. Dimethylformamide/Water Solvate)

A solution of lestaurtinib in N,N dimethylformamide was allowed to slowly evaporate to dryness under ambient conditions. The rate of evaporation was constrained by use of air tight film covers containing small holes. There are four independent molecules of lestaurtinib, two molecules of N,N. dimethylformamide and one molecule of water. One of the molecules of lestaurtinib, molecule A, and one of the molecules of solvent are disordered. The disordered N,N. dimethylformamide is hydrogen bound to molecule A the disordered lestaurtinib. The disorder is part of the hydrogen bonding pattern.

| Crystal system | Monoclinic | |
|---|---|---|
| Space group | P 2₁ | |
| Unit cell dimensions, Mo | a = 13.4876(18) Å | α = 90° |
| | b = 22.886(3) Å | β = 113.040(14)° |
| | c = 15.8170(14) Å | γ = 90° |
| Volume | 4492.9(9) Å³ | |
| Z | 2 | |

### Example 35

### Lestaurtinib Single Crystal Form XVI (2-Pentanone/Water Solvate)

A solution of lesaturtinib in 2-pentanone was allowed to slowly evaporate to dryness under ambient conditions. The rate of evaporation was constrained by use of air tight film covers containing small holes. The resulting data was sufficient to get molecular connectivity and to show the presence of 2-pentanone and water in this sample.

| Crystal system | Orthorhombic | |
|---|---|---|
| Space group | P2₁2₁2₁ | |
| Unit cell dimensions, Mo | a = 13.9300(6) Å | α = 90° |
| | b = 15.5209(7)Å | β = 90° |
| | c = 21.7861(9) Å | γ = 90° |
| Volume | 4710.3(4) Å³ | |
| Z | 4 | |

It is meant to be understood that peak heights in a XRPD spectrum may vary and will be dependent on variables such as the temperature, crystal size or morphology, sample preparation, or sample height in the analysis well of the Bruker AXS C2 GADDS or PANalytical X'Pert Pro X-Ray Diffraction Pattern Systems.

It is also meant to be understood that peak positions may vary when measured with different radiation sources. For example, Cu—Kα₁, Mo—Kα, Co-Kα and Fe—Kα radiation, having wavelengths of 1.54060 Å, 0.7107 Å, 1.7902 Å and 1.9373 Å, respectively, may provide peak positions that differ from those measured with Cu—Kα radiation.

The term “about” preceding a series of peak positions is meant to include all of the peak positions of the group which it precedes. For example, the phrase “about 6.8°, 8.5°, 9.7°, 12.0° or 13.2°” means “about 6.8°, about 8.5°, about 9.7°, about 12.0° or about 13.2°”.

In addition, the term “about” preceding a series of peak positions also means that all of the peaks of the group which it precedes are reported in terms of angular positions with a variability of ±0.2°. For example, “about 6.8°, 8.5°, 9.7°, 12.0° or 13.2°” means “6.8°±0.2°, 8.5°±0.2°, 9.7°±0.2°, 12.0°±0.2° or 13.2°±0.2+”.

As those skilled in the art will appreciate, numerous modifications and variations of the present invention are possible in view of the above teachings. It is therefore understood that within the scope of the appended claims, the invention can be practiced otherwise than as specifically described herein, and the scope of the invention is intended to encompass all such variations.

What is claimed:

1. A co-crystal comprising lestaurtinib and a second component selected from the group consisting of maleic acid, malonic acid, oxalic acid, glutaric acid, hippuric acid and urea.

2. The co-crystal of claim 1, wherein the co-crystal comprises lestaurtinib and maleic acid, and is characterized by a powder X-ray diffraction pattern comprising one or more peaks selected from the group consisting of about 7.56, 8.19, 16.47, 25.90 and 26.70 degrees 2-theta.

3. The co-crystal of claim 1, wherein the co-crystal comprises lestaurtinib and malonic acid, and is characterized by a powder X-ray diffraction pattern comprising one or more peaks selected from the group consisting of about 7.99, 15.16, 16.04, 26.11 and 27.17 degrees 2-theta.

4. The co-crystal of claim 1, wherein the co-crystal comprises lestaurtinib and oxalic acid, and is characterized by a powder X-ray diffraction pattern comprising one or more peaks selected from the group consisting of about 6.18, 7.44, 14.96, 20.19 and 25.78 degrees 2-theta.

5. The co-crystal of claim 1, wherein the co-crystal comprises lestaurtinib and glutaric acid, and is characterized by a powder X-ray diffraction pattern comprising one or more peaks selected from the group consisting of about 14.10, 14.60, 25.12, 25.56 and 26.55 degrees 2-theta.

6. The co-crystal of claim 1, wherein the co-crystal comprises lestaurtinib and hippuric acid, and is characterized by a powder X-ray diffraction pattern comprising one or more peaks selected from the group consisting of about 6.77, 14.23, 18.44, 20.61 and 25.19 degrees 2-theta.

7. The co-crystal of claim 1, wherein the co-crystal comprises lestaurtinib and urea, and is characterized by a powder X-ray diffraction pattern comprising one or more peaks selected from the group consisting of about 14.63, 22.24, 25.19, 25.86 and 26.56 degrees 2-theta.

8. The co-crystal of claim 1 for use as a pharmaceutical composition, comprising said co-crystal and one or more pharmaceutically acceptable excipients, diluents or carriers.

9. A pharmaceutical composition comprising Lestaurtinib Crystalline Form VI, Lestaurtinib Crystalline Form VII, Lestaurtinib Crystalline Form VIII, Lestaurtinib Crystalline Form IX, Lestaurtinib Crystalline Form X, Lestaurtinib Crystalline Form XI, Lestaurtinib Crystalline Form XII, Lestaurtinib Crystalline Form XIV, Lestaurtinib Crystalline Form XV, Lestaurtinib Crystalline Form XVI, Lestaurtinib Crystalline Form XX, Lestaurtinib Crystalline Form XXI, Lestaurtinib Crystalline Form XXII, Lestaurtinib Crystalline Form XXIII, Lestaurtinib Crystalline Form XXIV, Lestaurtinib Crystalline Form XXV, Lestaurtinib Crystalline Form XXVI, Lestaurtinib Crystalline Form XXVII, Lestaurtinib Crystalline Form XXVIII, or a mixture thereof.

10. The pharmaceutical composition of claim 9, wherein the lestaurtinib is Lestaurtinib Crystalline Form VI.

11. The pharmaceutical composition of claim 9, wherein the lestaurtinib is Lestaurtinib Crystalline Form VII.

12. The pharmaceutical composition of claim 9, wherein the lestaurtinib is Lestaurtinib Crystalline Form VIII.

13. The pharmaceutical composition of claim 9, wherein the lestaurtinib is Lestaurtinib Crystalline Form IX.

14. The pharmaceutical composition of claim 9, wherein the lestaurtinib is Lestaurtinib Crystalline Form X.

15. The pharmaceutical composition of claim 9, wherein the lestaurtinib is Lestaurtinib Crystalline Form XI.

16. The pharmaceutical composition of claim 9, wherein the lestaurtinib is Lestaurtinib Crystalline Form XII.

17. The pharmaceutical composition of claim 9, wherein the lestaurtinib is Lestaurtinib Crystalline Form XIV.

18. The pharmaceutical composition of claim 9, wherein the lestaurtinib is Lestaurtinib Crystalline Form XV.

19. The pharmaceutical composition of claim 9, wherein the lestaurtinib is Lestaurtinib Crystalline Form XVI.

20. The pharmaceutical composition of claim 9, wherein the Lestaurtinib is Lestaurtinib Crystalline Form XX.

21. The pharmaceutical composition of claim 9, wherein the lestaurtinib is Lestaurtinib Crystalline Form XXI.

22. The pharmaceutical composition of claim 9, wherein the lestaurtinib is Lestaurtinib Crystalline Form XXII.

23. The pharmaceutical composition of claim 9, wherein the lestaurtinib is Lestaurtinib Crystalline Form XXIII.

24. The pharmaceutical composition of claim 9, wherein the lestaurtinib is Lestaurtinib Crystalline Form XXIV.

25. The pharmaceutical composition of claim 9, wherein the lestaurtinib is Lestaurtinib Crystalline Form XXV.

26. The pharmaceutical composition of claim 9, wherein the lestaurtinib is Lestaurtinib Crystalline Form XXVI.

27. The pharmaceutical composition of claim 9, wherein the lestaurtinib is Lestaurtinib Crystalline Form XXVII.

28. The pharmaceutical composition of claim 9, wherein the lestaurtinib is Lestaurtinib Crystalline Form XXVIII.

29. The pharmaceutical composition of claim 9, further comprising amorphous lestaurtinib.

30. A solvate form of lestaurtinib that is Crystalline Form VI, Crystalline Form VII, Crystalline Form VIII, Crystalline Form IX, Crystalline Form X, Crystalline Form XI, Crystalline Form XII, Crystalline Form XIV, Crystalline Form XV, Crystalline Form XVI, Crystalline Form XX, Crystalline Form XXI, Crystalline Form XXII, Crystalline Form XXIII, Crystalline Form XXIV, Crystalline Form XXV, Crystalline Form XXVI, Crystalline Form XXVII, Crystalline Form XXVIII, or a mixture thereof.

31. The solvate of claim 30, wherein the solvate is Crystalline Form VI, and is characterized by a powder X-ray diffraction pattern comprising one or more peaks selected from the group consisting of about 14.23, 17.69, 25.79, 26.59 and 27.12 degrees 2-theta.

32. The solvate of claim 30, wherein the solvate is Crystalline Form VII, and is characterized by a powder X-ray diffraction pattern comprising one or more peaks selected from the group consisting of about 7.58, 17.75, 17.96, 21.48 and 22.08 degrees 2-theta.

33. The solvate of claim 30, wherein the solvate is Crystalline form VIII, and is characterized by a powder X-ray diffraction pattern comprising one or more peaks selected from the group consisting of about 7.70, 11.94, 12.05, 17.11, 17.62 and 18.05 degrees 2-theta.

34. The solvate of claim 30, wherein the solvate is Crystalline Form IX, and is characterized by a powder X-ray diffraction pattern comprising one or more peaks selected from the group consisting of about 7.79, 12.11, 15.55, 17.83 and 21.50 degrees 2-theta.

35. The solvate of claim 30, wherein the solvate is Crystalline Form X, and is characterized by a powder X-ray diffraction pattern comprising one or more peaks selected from the group consisting of about 7.69, 11.99, 15.46, 17.79 and 17.96 degrees 2-theta.

36. The solvate of claim 30, wherein the solvate is Crystalline Form XI, and is characterized by a powder X-ray diffraction pattern comprising one or more peaks selected from the group consisting of about 6.71, 14.44, 25.61, 26.51 and 27.80 degrees 2-theta.

37. The solvate of claim 30, wherein the solvate is Crystalline Form XII, and is characterized by a powder X-ray diffraction pattern comprising one or more peaks selected from the group consisting of about 7.15, 18.18, 18.77, 21.27 and 24.98 degrees 2-theta.

38. The solvate of claim 30, wherein the solvate is Crystalline Form XIV, and is characterized by a powder X-ray diffraction pattern comprising one or more peaks selected from the group consisting of about 7.75, 13.19, 14.21, 14.67, 17.55 and 25.13 degrees 2-theta.

39. The solvate of claim 30, wherein the solvate is Crystalline Form XV, and is characterized by a powder X-ray diffraction pattern comprising one or more peaks selected from the group consisting of about 11.05, 13.91, 17.04, 17.09 and 25.59 degrees 2-theta.

40. The solvate of claim 30, wherein the solvate is Crystalline Form XVI, and is characterized by a powder X-ray diffraction pattern comprising one or more peaks selected from the group consisting of about 8.12, 8.18, 10.31, 10.37 and 17.49 degrees 2-theta.

41. The solvate of claim 30, wherein the solvate is Crystalline Form XX, and is characterized by a powder X-ray diffraction pattern comprising one or more peaks selected from the group consisting of about 7.73, 15.46, 17.95, 18.07 and 22.06 degrees 2-theta.

42. The solvate of claim 30, wherein the solvate is Crystalline Form XXI, and is characterized by a powder X-ray diffraction pattern comprising one or more peaks selected from the group consisting of about 7.74, 12.19, 15.48, 18.18 and 22.27 degrees 2-theta.

43. The solvate of claim 30, wherein the solvate is Crystalline Form XXII, and is characterized by a powder X-ray diffraction pattern comprising one or more peaks selected from the group consisting of about 11.04, 13.60, 15.74, 17.04, 25.58 degrees 2-theta.

44. The solvate of claim 30, wherein the solvate is Crystalline Form XXIII, and is characterized by a powder X-ray diffraction pattern comprising one or more peaks selected from the group consisting of about 7.03, 14.06, 14.61, 15.04 and 26.31 degrees 2-theta.

45. The solvate of claim 30, wherein the solvate is Crystalline Form XXIV, and is characterized by a powder X-ray diffraction pattern comprising one or more peaks selected from the group consisting of about 7.66, 14.40, 14.54, 14.78 and 25.32 degrees 2-theta.

46. The solvate of claim 30, wherein the solvate is Crystalline Form XXV, and is characterized by a powder X-ray diffraction pattern comprising one or more peaks selected from the group consisting of about 5.52, 8.35, 10.88, 11.51 and 16.28 degrees 2-theta.

47. The solvate of claim 30, wherein the solvate is Crystalline Form XXVI, and is characterized by a powder X-ray diffraction pattern comprising one or more peaks selected from the group consisting of about 7.14, 13.00, 14.27, 16.58, 18.02 and 19.94 degrees 2-theta.

48. The solvate of claim 30, wherein the solvate is Crystalline Form XXVII, and is characterized by a powder X-ray diffraction pattern comprising one or more peaks selected from the group consisting of about 7.63, 9.80, 12.35, 15.27 and 21.93 degrees 2-theta.

49. The solvate of claim 30, wherein the solvate is Crystalline Form XXVIII, and is characterized by a powder X-ray diffraction pattern comprising one or more peaks selected from the group consisting of about 9.86, 13.95, 18.52, 19.76 and 25.43 degrees 2-theta.

50. A pharmaceutical composition comprising Lestaurtinib Crystalline Form XVII, Lestaurtinib Crystalline Form XVIII, Lestaurtinib Crystalline Form XIX, or a mixture thereof.

51. The pharmaceutical composition of claim 50 wherein the lestaurtinib is Lestaurtinib Crystalline Form XVII.

52. The pharmaceutical composition of claim 50 wherein the lestaurtinib is Lestaurtinib Crystalline Form XVIII.

53. The pharmaceutical composition of claim 50 wherein the lestaurtinib is Lestaurtinib Crystalline Form XIX.

54. The pharmaceutical composition of claim 50, further comprising amorphous lestaurtinib.

55. A crystalline anhydrate form of lestaurtinib that is Crystalline Form XVII, Crystalline Form XVIII, Crystalline Form XIX, or a mixture thereof.

56. The crystalline anhydrate of claim 55, wherein the crystalline anhydrate is Crystalline Form XVII, and is characterized by a powder X-ray diffraction pattern comprising one or more peaks selected from the group consisting of about 7.90, 15.76, 19.63, 19.70 and 20.07 degrees 2-theta.

57. The crystalline anhydrate of claim 55, wherein the crystalline anhydrate is Crystalline Form XVIII, and is characterized by a powder X-ray diffraction pattern comprising one or more peaks selected from the group consisting of about 7.76, 13.13, 15.64, 19.53 and 19.95 degrees 2-theta.

58. The crystalline anhydrate of claim 55, wherein the crystalline anhydrate is Crystalline Form XIX, and is characterized by a powder X-ray diffraction pattern comprising one or more peaks selected from the group consisting of about 9.61, 11.07, 15.71, 17.07 and 18.39 degrees 2-theta.

59. A pharmaceutical composition comprising Lestaurtinib Crystalline Form XIII.

60. The pharmaceutical composition of claim 59, further comprising amorphous lestaurtinib.

61. A crystalline hemihydrate of lestaurtinib that is Crystalline Form XIII.

62. The crystalline hemihydrate of claim 61, characterized by a powder X-ray diffraction pattern comprising one or more peaks selected from the group consisting of about 6.89, 14.26, 14.73, 16.95 and 17.58 degrees 2-theta.

63. A method of treating leukemia comprising administering to a patient in need thereof a therapeutically effective amount of a preparation prepared from a composition according to claim 9.

64. The method of claim 63, wherein the leukemia is selected from the group consisting of acute myeloid leukemia, chronic myeloid leukemia, acute lymphocytic leukemia and chronic lymphocytic leukemia.
